(19) United States
(12) Patent Application Publication
(10) Pub. No.: US 2025/0121104 A1
(43) Pub. Date: Apr. 17, 2025

(54) EGFR-CMET-TARGETED COMPOUNDS AND USES THEREOF

(71) Applicants: FUSION PHARMACEUTICALS INC., Hamilton (CA); ASTRAZENECA UK LIMITED, Cambridge (GB)

(72) Inventors: Sadaf AGHEVLIAN, Etobicoke (CA); Natalie GRINSHTEIN, Bothell, WA (US); Ian R. DUFFY, Oakville (CA); Thomas I. KOSTELINK, Hamilton (CA); Andrew Grier BUCHANAN, Cambridge (GB); Yariv MAZOR, Gaithersburg, MD (US); Srinath KASTURIRANGAN, Gaithersburg, MD (US); Qun DU, Gaithersburg, MD (US); Chunning YANG, Gaithersburg, MD (US); Frank Irvine COMER, Gaithersburg, MD (US)

(21) Appl. No.: 18/721,647

(22) PCT Filed: Dec. 20, 2022
(86) PCT No.: PCT/US2022/082021

§ 371 Date: Jun. 18, 2024

Related U.S. Application Data
(60) Provisional application No. 63/291,910, filed on Dec. 20, 2021.

(51) Int. Cl. A61K 51/10; A61K 45/06; A61P 35/00; C07K 16/28

(52) U.S. Cl. CPC A61K 51/1093; A61K 45/06; A61K 51/103; A61K 51/109; A61P 35/00; C07K 16/2863; A61K 2121/00

(57) ABSTRACT

Compounds, e.g., radioimmunoconjugates, including a chelating moiety or a metal complex thereof, a linker, and an antibody or antigen-binding fragment thereof targeting both EGFR and cMET. Pharmaceutical compositions of such compounds and methods of treatment for conditions, e.g., cancer, using such compounds or pharmaceutical compositions.

### RELATED APPLICATIONS

The present application claims priority to U.S. Provisional Patent Application No. 63/291,910, filed Dec. 20, 2021 the entire contents of which are hereby incorporated by reference for all purposes.

### BACKGROUND

Non-small cell lung cancer (NSCLC) remains the leading cause of cancer-related mortality worldwide. While with the development of EGFR-tyrosine kinase inhibitors (TKIs) the prognosis of advanced NSCLC has significantly improved for certain patients, metastatic NSCLC patients with long-term survival are still rare. A recent study by Lin et al., has shown that the prevalence of 5-year survival among patients with EGFR-mutant metastatic NSCLC treated with erlotinib or gefitinib is only 14.6%. Anti-EGFR TKIs are susceptible to resistance and the underlying resistant mechanism is long known to be the coexistence of EGFR mutation and cMET overexpression. One promising approach being tested in clinic is amivantamab, a human EGFR-cMET bispecific antibody that has been approved for treating non-small cell lung cancer. Targeting EGFR and cMET receptors increase the specificity of the treatment to cancer cells.

In general, the efficacy of antibodies depends on the number of targeted cells. Further, drug resistance is still a major challenge facing targeted cancer therapies, with de novo and acquired resistance limiting the long-term efficacy of targeted therapy. Mechanisms of resistance include secondary mutations, activation of oncogenic downstream signaling modules, ligand upregulation, and amplification of alternate growth factor receptors.

Thus, there remains a need for improved therapeutics (e.g., cancer therapeutics) that can target both EGFR and cMET without the above drawbacks.

### SUMMARY

The present disclosure relates to compounds (e.g., radioimmunoconjugates) that target both EGFR and cMET, pharmaceutical compositions thereof, and methods of treating cancer using such compounds or pharmaceutical compositions. Unlike naked antibodies, radioimmunoconjugates do not need to block the receptor function to have therapeutic efficacy; instead they emit radioactive particles (e.g., alpha emitters) that target the surrounding tumor cells within the limited range, thus preventing any off target associated toxicity. EGFR-cMET-targeted compounds (e.g., radioimmunoconjugates) used for EGFR and cMET overexpressing cancers utilize the ability of EGFR-cMET complex to undergo antibody triggered internalization to deliver the targeted radionuclides inside the cancer cells specifically. Antibody monovalent binding to either of the receptors on normal healthy tissues/cells does not lead to internalization.

In certain embodiments, provided compounds (e.g., radioimmunoconjugates) exhibit an increased excretion rate (e.g., after being administered to a mammal) compared to some currently known radiotherapeutics, while still maintaining therapeutic efficacy. In some embodiments, a faster excretion may limit off-target toxicities by limiting the amount of time that the compound stays in a subject. Thus, in some embodiments, provided compounds exhibit reduced off-target toxicities.

In one aspect, provided are compounds comprising the following structure, or pharmaceutically acceptable salts thereof:

A-L¹-(L²)ₙ-B Formula I

wherein
A is a chelating moiety or a metal complex thereof;
B is an antibody or antigen-binding fragment thereof,
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
L² each independently has the structure of Formula II:

—X¹-L³-Z¹— Formula II
wherein
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—, or —NR¹—, wherein “*” indicates the attachment point to L³, and R¹ is hydrogen, optionally substituted C₁-C₆ alkyl, optionally substituted C₁-C₆ heteroalkyl, optionally substituted aryl, or optionally substituted heteroaryl;
L³ is optionally substituted C₁-C₅₀ alkyl or optionally substituted C₁-C₅₀ heteroalkyl; and
Z¹ is —CH₂-#, —C(O)-#, —C(S)-#, —OC(O)-#, —C(O)O-#, —NR²C(O)-#, —C(O)NR²-#, or —NR²-#, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl, and
   wherein the antibody or antigen-binding fragment thereof comprises a first antigen-binding domain that is capable of binding epidermal growth factor receptor (EGFR) and a second antigen-binding domain that is capable of binding cMET,
   wherein the first antigen-binding domain comprises:
      i. a heavy chain variable (VH) region comprising the following complementarity determining regions (CDRs):
         HCDR1 having the amino acid sequence of SEQ ID NO: 1
         HCDR2 having the amino acid sequence of SEQ ID NO: 2
         HCDR3 having the amino acid sequence of SEQ ID NO: 3,
      or a variant thereof in which one or two or three amino acids in one or more of HCDR1, HCDR2, and HCDR3 are substituted with another amino acid; and
      ii. a light chain variable (VL) region comprising the following CDRs:
         LCDR1 having the amino acid sequence of SEQ ID NO: 4
         LCDR2 having the amino acid sequence of SEQ ID NO: 5
         LCDR3 having the amino acid sequence of SEQ ID NO: 6,
      or a variant thereof in which one or two or three amino acids in one or more of LCDR1, LCDR2, and LCDR3 are substituted with another amino acid.

In some embodiments, variable A in Formula I is a chelating moiety selected from the group consisting of DOTA (1,4,7,10-tetraazacyclododecane-1,4,7,10-tetraacetic acid), DOTMA (1R,4R,7R,10R)-α, α′, α″, α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10-tetra propionic acid), DO3AM-acetic acid (2-(4,7,10-tris(2-amino-2-oxoethyl)-1,4,7,10-tetraazacyclododecan-1-yl)acetic acid), DOTA-GA anhydride (2,2′,2″-(10-(2,6-dioxotetrahydro-2H-pyran-3-yl)-1,4,7,10-tetraazacyclododecane-1,4,7-triyl)triacetic acid, DOTP (1,4,7,10-tetraazacyclododecane-1,4,7,10-tetra(methylene phosphonic acid)), DOTMP (1,4,6,10-tetraazacyclodecane-1,4,7,10-tetramethylene phosphonic acid, DOTA-4AMP (1,4,7,10-tetraazacyclododecane-1,4,7,10-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacyclotetradecane-1,4,8,11-tetrapropionic acid), TETA (1,4,8,11-tetraazacyclotetradecane-1,4,8,11-tetra acetic acid), HEHA (1,4,7,10,13,16-hexaazacyclohexadecane-1,4,7,10,13,16-hexaacetic acid), PEPA (1,4,7,10,13-pentaazacyclopentadecane-N,N′,N″,N″′,N″″′-pentaacetic acid), H₄octapa (N,N′-bis(6-carboxy-2-pyridylmethyl)-ethylenediamine-N,N′-diacetic acid), H₂dedpa (1,2-[[6-(carboxy)-pyridin-2-yl]-methylamino]ethane), H₆phospa (N,N′-(methylenephosphonate)-N,N′-[6-(methoxycarbonyl)pyridin-2-yl]-methyl-1,2-diaminoethane), TTHA (triethylenetetramine-N,N,N′,N″,N″′,N″″-hexaacetic acid), DO2P (tetraazacyclododecane dimethanephosphonic acid), HP-DO3A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), and porphyrin.

In certain embodiments, variable A in Formula I is DOTA (1,4,7,10-tetraazacyclododecane-1,4,7,10-tetraacetic acid) or a metal complex thereof.

In some embodiments, the compound is represented by Formula I-a, or a metal complex thereof:

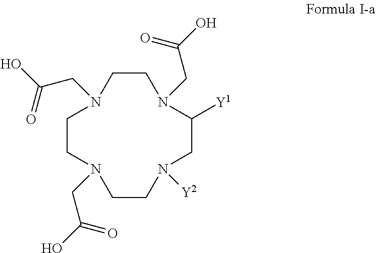

wherein Y¹ is —CH₂OCH₂(L²)ₙ-B, —C(O)(L²)ₙ-B, or —C(S)(L²)ₙ-B and Y² is —CH₂CO₂H; or
wherein Y¹ is H and Y² is L¹-(L²)ₙ-B. In certain embodiments, Y¹ is H.

In some embodiments, L¹ is

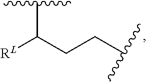

and R^{L} is hydrogen or —CO₂H.

In certain embodiments, X¹ is —C(O)NR¹—* or —NR¹C(O)—*, “*” indicating the attachment point to L³, and R¹ is H.

In certain embodiments, Z¹ is —CH₂—.

In some embodiments, L³ comprises (CH₂CH₂O)₂₋₂₀. In some embodiments, L³ is (CH₂CH₂O)ₘ(CH₂)_{w}, wherein m and w are each independently an integer between 0 and 10 (inclusive), and at least one of m and w is not 0.

In some embodiments, the metal complex comprises a metal selected from the group consisting of Bi, Pb, Y, Mn, Cr, Fe, Co, Zn, Ni, Tc, In, Ga, Cu, Re, a lanthanide, and an actinide. In some embodiments, the metal complex comprises a radionuclide selected from the group consisting of ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁸⁹Zr, ⁹⁰Y, ⁹⁷Ru, ⁹⁹Tc, ⁹⁹mTc, ¹⁰⁵Rh, ¹⁰⁹Pd, ¹¹¹n, ^{117m}Sn, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰¹Tl, ²⁰³Pb, ²¹¹At, ²¹²Pb, ²¹²Bi, ²¹³Bi, ²²³Ra, ²²⁵Ac, ²²⁷Th, and ²²⁹Th.

In some embodiments, variable A is a metal complex of a chelating moiety. In some such embodiments, the metal complex comprises a radionuclide. In some embodiments, the radionuclide is an alpha emitter, e.g., an alpha emitter selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (¹⁴⁹Tb), or a progeny thereof. In some embodiments, the radionuclide is ⁶⁸Ga, ¹¹¹In, ¹⁷⁷Lu, or ²²⁵Ac. In some embodiments, the radionuclide is ²²⁵Ac or a progeny thereof.

In some embodiments, compounds of Formula I comprise one of the following structures, or a metal complex thereof:

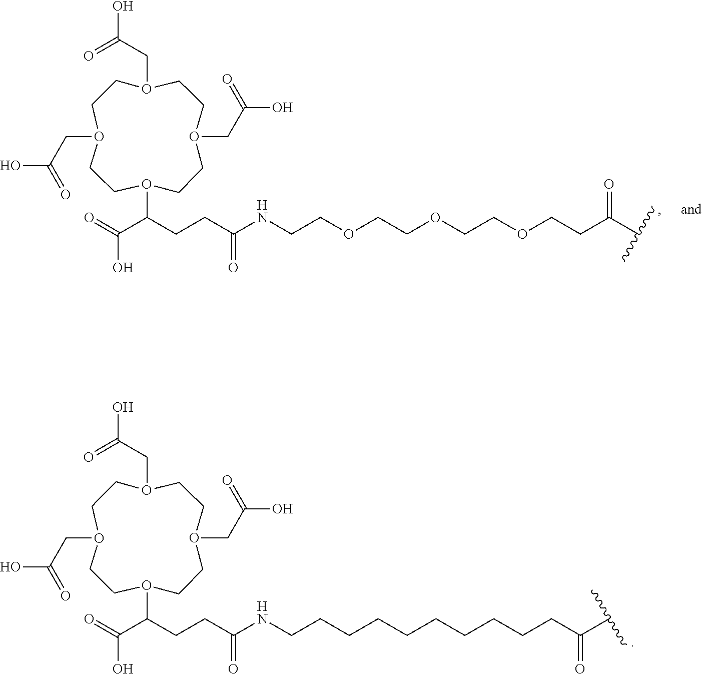

In some embodiments, the compound or a pharmaceutically acceptable salt thereof comprises the following structure, or a metal complex thereof:

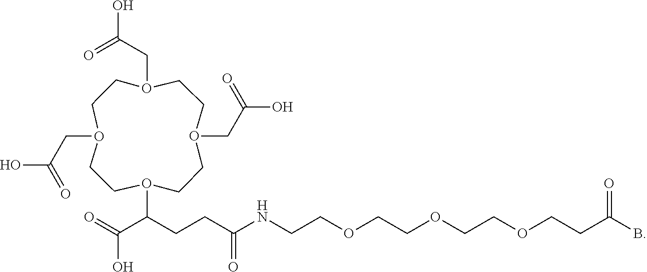

In some embodiments, referring to Formula I, variable A is a metal complex of a chelating moiety, and the metal complex comprises a radionuclide. In certain embodiments, the radionuclide is ⁶⁸Ga, ¹¹¹In, ¹⁷⁷Lu, or ²²⁵Ac. In certain embodiments, the radionuclide is ²²⁵Ac. In certain embodiments, the radionuclide is an alpha emitter selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (¹⁴⁹Tb), or a progeny thereof. In certain embodiments, the alpha emitter is ²²⁵Ac or a progeny thereof.

In some embodiments, referring to the antibody or antigen-binding fragment thereof included in the compounds of Formula I, the first antigen binding domain comprises:
i. a heavy chain variable (VH) region comprising the following complementarity determining regions (CDRs):
   HCDR1 having the amino acid sequence of SEQ ID NO: 1
   HCDR2 having the amino acid sequence of SEQ ID NO: 2
   HCDR3 having the amino acid sequence of SEQ ID NO: 3; and
ii. a light chain variable (VL) region comprising the following CDRs:
   LCDR1 having the amino acid sequence of SEQ ID NO: 4
   LCDR2 having the amino acid sequence of SEQ ID NO: 5
   LCDR3 having the amino acid sequence of SEQ ID NO: 6.

In some embodiments, referring to the antibody or antigen-binding fragment thereof included in the compounds of Formula I, the first antigen binding domain comprises:
a. a VH region comprising an amino acid sequence having at least 70%, at least 80%, at least 90%, or at least 95% sequence identity to the amino acid sequence of SEQ ID NO: 15; and
b. a VL region comprising an amino acid sequence having at least 70%, at least 80%, at least 90%, or at least 95% sequence identity to the amino acid sequence of SEQ ID NO: 16.

In some embodiments, referring to the antibody or antigen-binding fragment thereof included in the compounds of Formula I, the second antigen binding domain comprises:
i. a VH region comprising the following CDRs:
   HCDR1 having the amino acid sequence of SEQ ID NO: 17
   HCDR2 having the amino acid sequence of SEQ ID NO: 18
   HCDR3 having the amino acid sequence of SEQ ID NO: 19,
   or a variant thereof in which one or two or three amino acids in one or more of HCDR1, HCDR2, and HCDR3 are substituted with another amino acid; and
ii. a VL region comprising the following CDRs:
   LCDR1 having the amino acid sequence of SEQ ID NO: 20
   LCDR2 having the amino acid sequence of SEQ ID NO: 21
   LCDR3 having the amino acid sequence of SEQ ID NO: 22,
   or a variant thereof in which one or two or three amino acids in one or more of LCDR1, LCDR2, and LCDR3 are substituted with another amino acid.

In some embodiments, referring to the antibody or antigen-binding fragment thereof included in the compounds of Formula I, the second antigen binding domain comprises:
i. a VH region comprising the following CDRs:
   HCDR1 having the amino acid sequence of SEQ ID NO: 17
   HCDR2 having the amino acid sequence of SEQ ID NO: 18
   HCDR3 having the amino acid sequence of SEQ ID NO: 19; and
ii. a VL region comprising the following CDRs:
   LCDR1 having the amino acid sequence of SEQ ID NO: 20
   LCDR2 having the amino acid sequence of SEQ ID NO: 21
   LCDR3 having the amino acid sequence of SEQ ID NO: 22.

In some embodiments, referring to the antibody or antigen-binding fragment thereof included in the compounds of Formula I, the second antigen binding domain comprises:
a. a VH region comprising an amino acid sequence having at least 70%, at least 80%, at least 90%, or at least 95% sequence identity to the amino acid sequence of SEQ ID NO: 31; and
b. a VL region comprising an amino acid sequence having at least 70%, at least 80%, at least 90%, or at least 95% sequence identity to the amino acid sequence of SEQ ID NO: 32.

In some embodiments, the antibody or antigen-binding fragment thereof comprises:
a. a first antigen-binding domain that is capable of binding epidermal growth factor receptor (EGFR), comprising:
   i. a heavy chain variable (VH) region comprising the following complementarity determining regions (CDRs):
      HCDR1 having the amino acid sequence of SEQ ID NO: 1
      HCDR2 having the amino acid sequence of SEQ ID NO: 2
      HCDR3 having the amino acid sequence of SEQ ID NO: 3; and
   ii. a light chain variable (VL) region comprising the following CDRs:
      LCDR1 having the amino acid sequence of SEQ ID NO: 4
      LCDR2 having the amino acid sequence of SEQ ID NO: 5
      LCDR3 having the amino acid sequence of SEQ ID NO: 6; and
b. a second antigen-binding domain that is capable of binding cMET, comprising:
   i. a VH region comprising the following CDRs:
      HCDR1 having the amino acid sequence of SEQ ID NO: 17
      HCDR2 having the amino acid sequence of SEQ ID NO: 18
      HCDR3 having the amino acid sequence of SEQ ID NO: 19; and
   ii. a VL region comprising the following CDRs:
      LCDR1 having the amino acid sequence of SEQ ID NO: 20
      LCDR2 having the amino acid sequence of SEQ ID NO: 21
      LCDR3 having the amino acid sequence of SEQ ID NO: 22.

In some embodiments, the antibody or antigen-binding fragment thereof comprises:
a. a first heavy chain, wherein the first heavy chain comprises the VH region of the first antigen-binding domain, and a first heavy chain constant (CH) region or a fragment thereof;
b. a first light chain, wherein the first light chain comprises the VL region of the first antigen-binding domain, and a first light chain constant (CL) region or a fragment thereof;
c. a second heavy chain, wherein the second heavy chain comprises the VH region of the second antigen-binding domain, and a second heavy chain constant (CH) region or a fragment thereof; and
d. a second light chain, wherein the second light chain comprises the VL region of the second antigen-binding domain, and a second light chain constant (CL) region or a fragment thereof.

In some embodiments, the first and second CH region each comprise an amino acid sequence having at least 70%, at least 80%, at least 90%, or at least 95% sequence identity to the amino acid sequence of SEQ ID NO: 33.

In some embodiments, the first and second heavy chain form a heterodimer, optionally wherein one of the first and second heavy chains comprises a cysteine (C) residue at position 354 and a tryptophan (W) residue at position 366 and the other heavy chain comprises a cysteine (C) residue at position 349, a valine (V) residue at position 407, a serine (S) at position 366 and an alanine (A) at position 368, wherein the numbering of the constant region is as per the EU index.

In some embodiments, the antibody or antigen-binding fragment thereof comprises:
a. a modified CH region, wherein the modified CH region comprises a substitution of a native non-cysteine amino acid to a cysteine amino acid; and
b. a modified corresponding CL region, wherein the modified CL comprises a substitution of a native non-cysteine amino acid to a cysteine amino acid, wherein either:
   i. the first heavy chain comprises the modified CH region and the first light chain comprises the modified corresponding CL region; or
   ii. the second heavy chain comprises the modified CH region and the second light chain comprises the modified corresponding CL region, and
   wherein the substituted cysteine of the modified CH region and the substituted cysteine of the modified corresponding light chain can form a disulfide bond.

In some embodiments, the modified CH region comprises a substitution of a native non-cysteine amino acid to a cysteine amino acid at position 126; and the modified corresponding CL region comprises a substitution of a native non-cysteine amino acid to a cysteine amino acid at position 121, wherein the numbering of the constant region is as per the EU index.

In some embodiments, the first and/or second CH region comprise a mutation to reduce or abrogate binding of the antibody or antigen-binding fragment thereof to one of more Fcγ receptors.

In some embodiments, the first and/or second CH region comprise a phenylalanine at position 234, glutamic acid at position 235, and serine at position 331, wherein the numbering of the constant region is as per the EU index.

In some embodiments, the compound of Formula I features that:
a. the first CH region comprises an amino acid sequence having at least 90%, 91%, 92%, 93%, 94%, 95%, 96%, 97%, 98%, 99%, or 100% sequence identity to the sequence set forth in SEQ ID NO: 39;
b. the second CH region comprises an amino acid sequence having at least 90%, 91%, 92%, 93%, 94%, 95%, 96%, 97%, 98%, 99%, or 100% sequence identity to the sequence set forth in SEQ ID NO: 40,
c. the first CL region comprises an amino acid sequence having at least 90%, 91%, 92%, 93%, 94%, 95%, 96%, 97%, 98%, 99%, or 100% sequence identity to the sequence set forth in SEQ ID NO: 41; and
d. the second CL region comprises an amino acid sequence having at least 90%, 91%, 92%, 93%, 94%, 95%, 96%, 97%, 98%, 99%, or 100% sequence identity to the sequence set forth in SEQ ID NO: 34.

In some embodiments, the antibody or antigen-binding fragment thereof comprises:
a. a first heavy chain comprising an amino acid sequence having at least 90%, 91%, 92%, 93%, 94%, 95%, 96%, 97%, 98%, 99%, or 100% sequence identity to the sequence set forth in SEQ ID NO: 35;
b. a second heavy chain comprising an amino acid sequence having at least 90%, 91%, 92%, 93%, 94%, 95%, 96%, 97%, 98%, 99%, or 100% sequence identity to the sequence set forth in SEQ ID NO: 36;
c. a first light chain comprising an amino acid sequence having at least 90%, 91%, 92%, 93%, 94%, 95%, 96%, 97%, 98%, 99%, or 100% sequence identity to the sequence set forth in SEQ ID NO: 37; and
d. a second light chain comprising an amino acid sequence having at least 90%, 91%, 92%, 93%, 94%, 95%, 96%, 97%, 98%, 99%, or 100% sequence identity to the sequence set forth in SEQ ID NO: 38.

In some embodiments, the compound of Formula I features that:
a. the first heavy chain comprises an amino acid sequence having the sequence set forth in SEQ ID NO: 35;
b. the second heavy chain comprises an amino acid sequence having the sequence set forth in SEQ ID NO: 36;
c. the first light chain comprises an amino acid sequence having the sequence set forth in SEQ ID NO: 37; and
d. the second light chain comprises an amino acid sequence having the sequence set forth in SEQ ID NO: 38.

In some embodiments, the compound of Formula I features that:
a. the first antigen-binding domain that is capable of binding EGFR is capable of binding cynomologus EGFR;
b. the first antigen-binding domain that is capable of binding EGFR is capable of binding mouse EGFR;
c. the second antigen-binding domain that is capable of binding cMET is capable of binding cynomologus cMET;
d. the first antigen-binding domain is specific for EGFR;
e. the second antigen-binding domain is specific for cMET;
f. the antibody or antigen-binding fragment thereof is capable of concurrently engaging EGFR and cMET;
g. the antibody or antigen-binding fragment thereof is capable of being internalised into a cell;
h. the antibody or antigen-binding fragment thereof has cytotoxic activity when measured in an in vitro cell viability assay; and/or
i. the antibody or antigen-binding fragment thereof is capable of blocking ligand dependent signalling of EGFR and/or cMET.

In some embodiments, the compound of Formula I features that the first antigen-binding domain is capable of binding human EGFR with an affinity having a Kd that is:
a. between 10 and 100 nM;
b. between 20 and 80 nM;
c. between 30 and 75 nM; or
d. between 35 and 50 nM.

In some embodiments, the compound of Formula I features that the second antigen-binding domain is capable of binding human cMET with an affinity having a Kd that is:
a. lower than 10 nM; or
b. lower than 5 nM.

In some embodiments, the compound of Formula I features that the first antigen-binding domain is capable of binding human EGFR with an affinity that is lower than the affinity that an antigen-binding domain comprising the variable heavy region sequence and variable light region sequence of antibody QD6, the sequences set forth in SEQ ID NOs: 47 and 48, respectively.

In some embodiments, the binding affinity described above is measured by surface plasmon resonance.

In some embodiments, the compound of Formula I comprises the following structure:

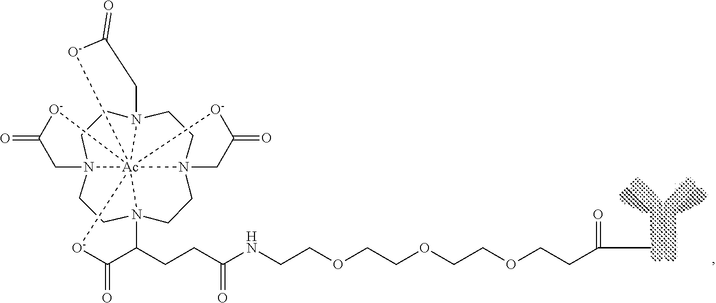

wherein

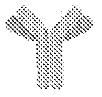

is an antibody or an antigen-binding fragment thereof that is capable of specifically binding both EGFR and cMET (e.g., EGFR-cMET monoclonal bispecific antibody RAA22/B09 DuetMab described below). In some embodiments, the antibody or an antigen-binding fragment thereof is linked to A-L¹-(L²)ₙ- via the side-chain amino group of a lysine residue.

In another aspect, the present disclosure also relates to a pharmaceutical composition comprising one of the compounds described above and a pharmaceutically acceptable carrier, diluent, or excipient.

Still within the scope of this invention is a method of treating cancer, said method comprising administering to a subject (e.g., a human) in need thereof a therapeutically effective amount a compound described above or a respective composition thereof.

In some embodiments, the cancer is a solid tumor cancer selected from the group consisting of adrenocortical carcinoma, bladder cancer, breast cancer, cervical cancer, colorectal cancer, endometrial adenocarcinoma, Ewing's sarcoma, gallbladder carcinoma, glioma, head and neck cancer (e.g., head and neck squamous cell carcinomas or HNSCC), liver cancer, lung cancer (e.g., non-small cell lung cancer or NSCLC), neuroblastoma, neuroendocrine cancer, ovarian cancer, pancreatic cancer (e.g., pancreatic ductal adenocarcinoma or PDAC), prostate cancer, gastric cancer, renal cell carcinoma, salivary adenoid cystic cancer, spermatocytic seminoma, and uveal melanoma.

In some embodiments, the cancer is lung cancer, colorectal cancer, pancreatic cancer, or head and neck cancer.

In some embodiments, the method of treatment of this invention further comprises administering to a subject (e.g., a human) in need thereof an antiproliferative agent, radiation sensitizer, an immunoregulatory or immunomodulatory agent.

Still within the scope of this disclosure is a compound or the pharmaceutical composition described above for use in a method of treatment of cancer.

The present disclosure further covers use of a compound or the pharmaceutical composition described above in the manufacture of a medicament for the treatment of cancer.

### BRIEF DESCRIPTION OF THE DRAWINGS

FIGS. 1A and 1B are graphical depiction of RAA22/B09-57 and QD6/B09-57 DuetMabs, respectively. Shown are the Fabs of anti-EGFR RAA22 and QD6, Fab of anti-cMET B09-57 and Hole and Knob heavy chains. The structural rendering is a composite of individual domain structures.

FIG. 2 shows concurrent binding studies using antigen capture format were performed by Octet analysis. Sensors loaded with human cMET antigen were exposed to successive association and dissociation interactions first with antibodies then with human EGFR antigen. Ass=association; Diss=dissociation; NI-NTA=Nickel-nitrilotriacetic acid.

FIG. 3A shows ELISA results showing EGFR and c-Met species cross reactivity. The high affinity monospecific EGFR IgG, QD6, as well as the monovalent bispecific EGFR/cMET DuetMAb, QD6/B09, bound to human, cynomolgus monkey, and mouse EGFR. The lowered affinity monospecific EGFR IgG, RAA22, bound more weakly to human, cynomolgus monkey, and mouse EGFR compared to QD6 and the corresponding monovalent bispecific EGFR/c-Met DuetMAb, RAA22/B09, showed still weaker binding to human and cynomolgus monkey EGFR relative to the bivalent parental IgG, RAA22, and nominal binding to mouse EGFR. The monospecific c-Met IgG, B09, as well as all of the bispecific variants, showed comparable binding to human and cynomolgus monkey c-Met but no detectable binding to mouse c-Met.

FIG. 3B shows ELISA results showing EGFR and c-Met family specificity. None of the antibodies tested showed any appreciable binding to any of the EGFR HER family proteins (HER2, HER3, or HER4) or any of the c-Met family members (Ron (CD136) or Semaphorin 3a).

FIG. 4 shows internalization of RAA22/B09 bispecific mAb and trafficking to acidified intracellular compartments was visualized using antibodies labeled with pHAb pH sensitive dye (Promega). Control antibodies included R347 isotype control and the monovalent bispecific control antibodies anti-EGFR RAA22/R347 and anti-cMET B09/R347. The pHAb labelled antibodies were incubated with NCI-H1975 lung cancer cells at a concentration of 1.25 μg/mL in a humidified incubator at 37° C. and 5% CO₂. Fluorescent images were captured at the indicated time points on an Operetta High Content Imaging system using the Cy3 filter. Increased cellular fluorescence intensity over time was deemed evidence of internalization and trafficking to acidic intracellular compartments as measured by the pH sensitive fluor.

FIG. 5 shows internalization of RAA22/B09 bispecific mAb and trafficking to acidified intracellular compartments was visualized using antibodies labeled with pHAb pH sensitive dye, but the cells were treated with antibodies at the lower concentration of 0.625 μg/mL.

FIG. 6A shows kinetics of QD6/B09 and RAA22/B09 monoclonal antibody (mAb) internalization in H1975 cells. (a) Image overlays of cells labeled with CellTracker Blue CMAC for cytoplasm (blue) and with 2.5 μg/mL of QD6/B09-AlexaFluor647 (magenta, top panel) or 2.5 μg/mL of RAA22/B09AlexaFluor-647 (magenta, bottom panel) at the start of internalization and 1 hr after. Cells were labeled with CellTracker Blue CMAC, then bound with mAbs-AlexaFuor6457 at 2-8° C. and subjected to internalization conditions: (37° C., 70% humidity and 5% CO₂).

FIG. 6B shows kinetics of QD6/B09 and RAA22/B09 monoclonal antibody (mAb) internalization in H1975 cells. Time course of mAb-AlexaFluor647 internalization determined by quantitative analysis of kinetic images using the algorithm (Material and Methods). Kinetic images taken at 5-minute intervals were processed using the algorithm (Vainshtein, 2015) to determine antibody accumulation in cytoplasm. Antibody signals in cytoplasm normalized to antibody fluorescence in the cell (fraction in cytoplasm) for QD6/B09-AlexaFluor647 (red) versus RAA22/B09-AlexaFluor647 (blue) are shown for one of the three independent experiments. Internalization rate constant (kint) was calculated from the time course of internalization using the curve fitting with the equation Fcyt(t)=(1−e^{-kint·t}) ·Fmax,cyt, where Fmax,cyt is the maximal ratio cytoplasmic intensity per cell to total intensity per cell. T ½ calculated from kint was 37.5±10.6 min for QD6/B09 and 43.2±15.5 for RAA22/B09 (from n=3).

FIG. 7A shows internalization profiles of QD6/B09 DuetMab and its respective single-arm control antibodies. Internalization profiles are displayed via time course of the respective membrane and cytoplasm signals for each construct. QD6/B09 set was acquired using an Opera confocal fluorescence microscope.

FIG. 7B shows internalization profiles of RAA22/B09 DuetMab and its respective single-arm control antibodies. Internalization profiles are displayed via time course of the respective membrane and cytoplasm signals for each construct. The set was acquired using a Zeiss spinning-disc confocal fluorescence microscope. Identical profiles for QD6/B09 and QD6/IgG indicate internalization mode driven by EGFR-arm of QD6/B09 DuetMab, whereas RAA22/B09 DuetMab requires engagement of both EGFR and c-MET arms for efficient internalization.

FIG. 8A shows internalization profile of RAA22/B09 in cells expressing moderate and high target c-MET and EGFR cell surface receptors. Shown are membrane, cytoplasm and total signals for RAA22/B09-AF647 in H1975 cells. One representative experiment of 2 is shown. H1975 cells show concurrent drop in total and membrane intensities indicating dissociation of antibody from the cell surface.

FIG. 8B is equivalent to FIG. 8A but in HCC827 cells. HCC827 cells have stable total signal over experimental time course. Decrease of membrane signal is derived antibody internalization.

FIG. 9A shows internalization of RAA22/B09 single arm control antibodies in HCC827 cells. Intensity profiles of the RAA22/IgG single arm is about 10-fold lower than RAA22/B09 due to weaker binding to EGFR through single arm binding

FIG. 9B shows that B09/IgG single arm dissociates from cell membrane as signaled by simultaneous drops in total and membrane signals over time.

FIG. 10A is a schematic depicting the general structure of bifunctional chelate comprising a chelate, a linker, and a cross-linking group.

FIG. 10B is a schematic depicting the general structure of a bifunctional conjugate comprising a chelate, a linker, and a targeting moiety.

FIG. 10C and FIG. 10D are schematics depicting the structures of [¹⁷⁷Lu]-DOTA-anti-EGFR-cMET and [²²⁵Ac]-DOTA-anti-EGFR-cMET, two exemplary EGFR-cMET radioimmunoconjugates disclosed herein.

FIG. 11 is a schematic depicting the synthesis of the bifunctional chelate, 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B). Synthesis of Compound B is described in EXAMPLE 6.

FIG. 12 is a schematic depicting the synthesis of the bifunctional chelate, 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic acid (Compound C). Synthesis of Compound C is described in EXAMPLE 7.

FIG. 13 is a schematic depicting the conjugation and radiolabeling for synthesis of [¹⁷⁷Lu]-Compound C-anti-EGFR-cMET conjugate (e.g., Compound E and Compound F described below). See EXAMPLE 8.

FIGS. 14A-14B show binding of Compound E to different cell lines with varying expression levels of EGFR and c-MET in the absence (total binding) or presence (nonspecific binding) of 4 μM unlabeled antibody. Specific binding was calculated by subtraction of nonspecific binding from total binding. Values shown are the mean±SEM (n=3). See EXAMPLE 9.

FIG. 15 shows the results of internalization (or in vitro residualization) of Compound E (10 nM) in HCC827 cells, HT29 cells, H441 cells, and H1975 cells with varying expression levels of EGFR and c-MET at 2 h and 24 h post incubation. Values shown are the mean±SEM (n=3). See EXAMPLE 10.

FIGS. 16A-16E show a plot representing the results of biodistribution studies in a various animal models and injected with Compound E (0.7 MBq/2 μg) in female balb/c mice (n=3) with subcutaneous xenografts at specified timepoints following intravenous injection. Percentage injected dose per gram of tissue (% ID/g) is plotted on the x-axis and is shown for blood, bone, brain, heart, intestines, kidneys, lungs, liver, pancreas, spleen, stomach, skin, urine and bladder, and tumor at 4, 24, 48, 96, and 168 hours. See EXAMPLE 11.

FIGS. 17A-17C show a plot representing the results of in vivo efficacy studies in various animal models in female balb/c mice (n=5) following intravenous injection of Compound F (50-400 nCi/2 μg), cold antibody and vehicle. See EXAMPLE 12.

### DETAILED DESCRIPTION

Radioimmunoconjugates are designed to target a protein or receptor that is upregulated in a disease state to deliver a radioactive payload to damage and kill cells of interest (radioimmunotherapy). Delivering a radioactive payload results in targeted alpha, beta, gamma particle or Auger electron emission that can cause direct effects to DNA (such as single or double stranded DNA breaks) or indirect effects such as by-stander or crossfire effects.

Radioimmunoconjugates typically contain a biological targeting moiety (e.g., an antibody or antigen binding fragment thereof that is capable of specifically binding to both EGFR and cMET), a radionuclide (e.g., an alpha or beta emitter), and a molecule that links the two. Conjugates are formed when a bifunctional chelate is appended to the biological targeting moiety so that structural alterations are minimal while maintaining target affinity. Once radiolabelled, the final radioimmunoconjugate is formed.

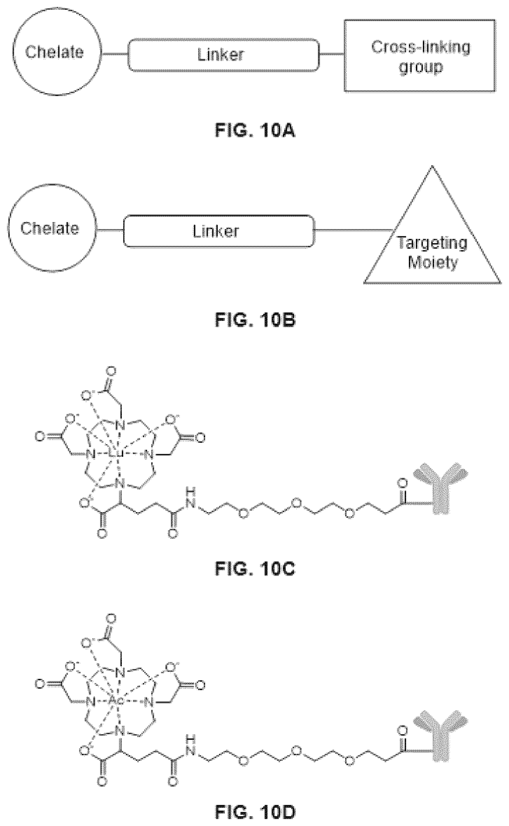

Bifunctional chelates structurally contain a chelate, a linker, and a cross-linking group (FIG. 10A). When developing new bifunctional chelates, most efforts focus on the chelating portion of the molecule. Several examples of bifunctional chelates have been described with various cyclic and acyclic structures conjugated to a targeted moiety. [Bioconjugate Chem. 2000, 11, 510-519; Bioconjugate Chem. 2012, 23, 1029-1039; Mol Imaging Biol. 2011, 13, 215-221, Bioconjugate Chem. 2002, 13, 110-115.]

One of the key factors of developing safe and effective radioimmunoconjugates is maximizing efficacy while minimizing off-target toxicity in normal tissue. While this statement is one of the core tenets of developing new drugs, the application to radioimmunotherapeutics presents new challenges. Radioimmunoconjugates do not need to block a receptor, as needed with a therapeutic antibody, or release the cytotoxic payload intracellularly, as required by an antibody drug conjugate (“ADC”), to have therapeutic efficacy. However, the emission of the toxic particle is an event that occurs as a result of first-order (radioactive) decay and can occur at random anywhere inside the body after administration. Once the emission occurs, damage could occur to surrounding cells within the range of the emission leading to the potential of off-target toxicity. Therefore, limiting exposure of these emissions to normal tissue is the key to developing new therapeutic radioimmunoconjugates.

One potential method for reducing off-target exposure is to remove the radioactivity more effectively from the body (e.g., from normal tissue in the body). One mechanism is to increase the rate of clearance of the biological targeting agent. This approach likely requires identifying ways to shorten the half-life of the biological targeting agent, which is not well described for biological targeting agents. Regardless of the mechanism, increasing drug clearance will also negatively impact the pharmacodynamics/efficacy in that the more rapid removal of drug from the body will lower the effective concentration at the site of action, which, in turn, would require a higher total dose and would not achieve the desired results of reducing total radioactive dose to normal tissue.

Other efforts have focused on accelerating the metabolism of the portion of the molecule that contains the radioactive moiety. To this end, some efforts have been made to increase the rate of cleavage of the radioactivity from the biological targeting agents using what have been termed “cleavable linkers”. Cleavable linkers, however, have been taken on different meaning as it relates to radioimmunoconjugates. Cornelissen, et al. has described cleavable linkers as those by which the bifunctional chelate attaches to the biologic targeting agent through a reduced cysteine, whereas others have described the use of enzyme-cleavable systems that require the co-administration of the radioimmunoconjugate with a cleaving agent/enzyme to release [Mol Cancer Ther. 2013, 12(11), 2472-2482; Methods Mol Biol. 2009, 539, 191-211; Bioconjug Chem. 2003, 14(5), 927-33]. These methods either change the nature of the biological targeting moiety, in the case of the cysteine linkage, or are not practical from a drug development perspective (enzyme cleavable systems) since, in the case of the citations provided, require the administration of two agents.

The present disclosure provides, among other things, compounds, e.g., radioimmunoconjugates, that are more effectively eliminated from the body after catabolism and/or metabolism, thereby more effectively eliminating radioactivity from the body while maintaining therapeutic efficacy. This unexpected superiority is achieved by making modifications to the linker region of the bifunctional chelate.

Disclosed immunoconjugates may, in some embodiments, achieve a reduction of total body radioactivity, for example, by increasing the extent of excretion of the catabolic/metabolic products while maintaining the pharmacokinetics of the intact molecule when compared to known bifunctional chelates. In some embodiments, this reduction in radioactivity results from the clearance of catabolic/metabolic by-products without impacting other in vitro and in vivo properties such as binding specificity (in vitro binding), cellular retention, and tumor uptake in vivo. Thus, in some embodiments, provided compounds achieve reduced radioactivity in the human body while maintaining on-target activity.

### Definitions

As used herein, the term “bind” or “binding” of a targeting moiety means an at least temporary interaction or association with or to a target molecule, e.g., human EGFR-cMET, as described herein.

The terms “bifunctional chelate,” as used herein, refers to a compound that comprises a chelate, a linker, and a cross-linking group. See, e.g., FIG. 10A. A “cross-linking group” is a reactive group that is capable of joining two or more molecules, e.g., joining a bifunctional chelate and a targeting moiety, by a covalent bond.

The term “bifunctional conjugate,” as used herein, refers to a compound that comprises a chelate or metal complex thereof, a linker, and a targeting moiety, e.g., an antibody or antigen-binding fragment thereof. See, e.g., FIG. 10B.

The term “cancer,” as used herein, refers to any cancer caused by the proliferation of malignant neoplastic cells, such as tumors, neoplasms, carcinomas, sarcomas, leukemias, and lymphomas. In some embodiments, a cancer of the present disclosure comprises cells (e.g., tumor cells) expressing EGFR and cMET, such as, but not limited to, lung cancer, colorectal cancer, pancreatic cancer, or head and neck cancer.

The term “chelate,” as used herein, refers to an organic compound or portion thereof that can be bonded to a central metal or radiometal atom at two or more points.

The term “conjugate,” as used herein, refers to a molecule that contains a chelating group or metal complex thereof, a linker group, and which optionally contains a targeting moiety, e.g., an antibody or antigen-binding fragment thereof.

As used herein, unless otherwise noted, the phrase “constant region,” when used in reference to an antibody or a fragment thereof (e.g., an IgG1, an IgG2, or an IgG4 constant region) is intended to encompass both wild type constant regions and variants (e.g., constant regions having at least 85%, at least 90%, at least 92%, at least 94%, at least 95%, at least 96%, at least 97%, at least 98%, or at least 99% amino acid sequence identity with a reference sequence for a wild-type constant region.

As used herein, the term “compound,” is meant to include all stereoisomers, geometric isomers, and tautomers of the structures depicted.

The compounds recited or described herein can be asymmetric (e.g., having one or more stereocenters). All stereoisomers, such as enantiomers and diastereomers, are intended unless otherwise indicated. Compounds discussed in the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis.

As used herein, “detection agent” refers to a molecule or atom which is useful in diagnosing a disease by locating the cells containing the antigen. Various methods of labeling polypeptides with detection agents are known in the art. Examples of detection agents include, but are not limited to, radioisotopes and radionuclides, dyes (such as with the biotin-streptavidin complex), contrast agents, luminescent agents (e.g., fluorescein isothiocyanate or FITC, rhodamine, lanthanide phosphors, cyanine, and near IR dyes), and magnetic agents, such as gadolinium chelates.

As used herein, the term “radionuclide,” refers to an atom capable of undergoing radioactive decay (e.g., ³H, ¹⁴C, ¹⁵N, ¹⁸F, ³⁵S, ⁴⁴Sc, ⁴⁷Sc, ⁵⁵Co, ⁶⁰Cu, ⁶¹Cu, ⁶²Cu, ⁶⁴Cu, ⁶⁷Cu, ⁷⁵Br, ⁷⁶Br, ⁷⁷Br, ⁸⁹Zr, ⁸⁶Y, ⁸⁷Y, ⁹⁰Y, ⁹⁷Ru, ⁹⁹Tc, ⁹⁹mTc, ¹⁰⁵Rh, ¹⁰⁹Pd, ¹¹¹In, ¹²³I, ¹²⁴I, ¹²⁵I, ¹³¹I, ¹⁴⁹Pm, ¹⁴⁹Tb ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰³Pb, ²¹¹At, ²¹²Pb, ²¹²Bi, ²¹³Bi, ²²³Ra, ²²⁵Ac, ²²⁷Th, ²²⁹Th, ⁶⁶Ga, ⁶⁷Ga, ⁶⁸Ga, ⁸²Rb, ^{117m}Sn, ²⁰¹Tl). The terms radioactive nuclide, radioisotope, or radioactive isotope may also be used to describe a radionuclide. Radionuclides may be used as detection agents, as described herein. In some embodiments, the radionuclide may be used as therapeutic agents, e.g., an alpha-emitting radionuclide.

The term an “effective amount” of an agent (e.g., any of the foregoing conjugates), as used herein, is that amount sufficient to effect beneficial or desired results, such as clinical results, and, as such, an “effective amount” depends upon the context in which it is being applied. For example, in therapeutic applications, an “effective amount” may be an amount sufficient to cure or at least partially arrest the symptoms of the disorder and its complications, and/or to substantially improve at least one symptom associated with the disease or a medical condition. Typically, an “effective amount” in the context of the present disclosure is an amount of a compound (e.g., radioimmunoconjugate) disclosed herein, e.g., an Ac-225-radioimmunoconjugate, that produces at least some measurable therapeutic response or desired effect in some fraction of the patient to whom it is administered. For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but may, for example, provide a treatment for a disease or condition such that the onset of the disease or condition is delayed, hindered, or prevented, such that the disease or condition symptoms are ameliorated, or such that the term of the disease or condition is changed. For example, the disease or condition may become less severe and/or recovery is accelerated in an individual. An effective amount may be administered by administering a single dose or multiple (e.g., at least two, at least three, at least four, at least five, or at least six) doses.

The term “immunoconjugate,” as used herein, refers to a conjugate that includes a targeting moiety, such as an antibody (or antigen-binding fragment thereof), nanobody, affibody, or a consensus sequence from Fibronectin type III domain. In some embodiments, the immunoconjugate comprises an average of at least 0.10 conjugates per targeting moiety (e.g., an average of at least 0.2, 0.3, 0.4, 0.5, 0.6, 0.7, 0.8, 0.9, 1, 2, 3, 4, 5, 6, 7, or 8 conjugates per targeting moiety).

The term “radioconjugate,” as used herein, refers to any conjugate that includes a radioisotope or radionuclide, such as any of the radioisotopes or radionuclides described herein.

The term “radioimmunoconjugate,” as used herein, refers to any immunoconjugate that includes a radioisotope or radionuclide, such as any of the radioisotopes or radionuclides described herein. A radioimmunoconjugate provided in the present disclosure typically refers to a bifunctional conjugate that comprises a metal complex formed from a radioisotope or radionuclide.

The term “radioimmunotherapy,” as used herein, refers a method of using a radioimmunoconjugate to produce a therapeutic effect. In some embodiments, radioimmunotherapy may include administration of a radioimmunoconjugate to a subject in need thereof, wherein administration of the radioimmunoconjugate produces a therapeutic effect in the subject. In some embodiments, radioimmunotherapy may include administration of a radioimmunoconjugate to a cell, wherein administration of the radioimmunoconjugate kills the cell. Wherein radioimmunotherapy involves the selective killing of a cell, in some embodiments the cell is a cancer cell in a subject having cancer.

The term “pharmaceutical composition,” as used herein, represents a composition containing a radioimunoconjugate described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, radioprotectants, sorbents, suspending or dispersing agents, sweeteners, or waters of hydration. Exemplary excipients include, but are not limited to: ascorbic acid, histidine, phosphate buffer, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts of the compounds described here that are, within the scope of sound medical judgment, suitable for use in contact with the tissues of humans and animals without undue toxicity, irritation, or allergic response. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, among others. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “polypeptide,” as used herein, refers to a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides can include one or more “non-natural” amino acids or other entities that nonetheless are capable of integrating into a polypeptide chain. In some embodiments, a polypeptide may be glycosylated, e.g., a polypeptide may contain one or more covalently linked sugar moieties. In some embodiments, a single “polypeptide” (e.g., an antibody polypeptide) may comprise two or more individual polypeptide chains, which may in some cases be linked to one another, for example by one or more disulfide bonds or other means.

By “subject” is meant a human or non-human animal (e.g., a mammal).

By “substantial identity” or “substantially identical” is meant a polypeptide sequence that has the same polypeptide sequence, respectively, as a reference sequence, or has a specified percentage of amino acid residues, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). Sequence identity may be measured using sequence analysis software on the default setting (e.g., Sequence Analysis Software Package of the Genetics Computer Group, University of Wisconsin Biotechnology Center, 1710 University Avenue, Madison, WI 53705). Such software may match similar sequences by assigning degrees of homology to various substitutions, deletions, and other modifications.

As used herein, and as well understood in the art, “to treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

As used herein, the term “about” or “approximately,” when used in reference to a quantitative value, includes the recited quantitative value itself, unless specifically stated otherwise. As used herein, the term “about” or “approximately” refers to a ±10% variation from the recited quantitative value unless otherwise indicated or inferred from the context.

As used herein, the term “targeting moiety” refers to any molecule or any part of a molecule that is capable of binding to a given target. The term, “EGFR-cMET targeting moiety” refers to a targeting moiety (e.g., an antibody or antigen-binding fragment thereof) that is capable of binding to both EGFR and cMET, e.g., anti-EGFR-cMET antibody.

As used herein, the term “fragment,” when used to refer to an EGFR or cMET fragment, refers to N-terminally and/or C-terminally truncated EGFR or cMET or protein domains thereof. Unless otherwise noted, fragments of EGFR or cMET used in accordance with embodiments described herein retain the capability of the full-length EGFR or cMET to be recognized and/or bound by an EGFR-cMET-targeting moiety as described in the present disclosure.

The use of the terms “a” and “an” and “the” and similar referents in the context of describing the disclosure (especially in the context of the claims) are to be construed to cover both the singular and the plural, unless otherwise indicated herein or clearly contradicted by context.

Unless specifically stated or obvious from context, as used herein the term “or” is understood to be inclusive and covers both “or” and “and”.

The term “and/or” where used herein is to be taken as specific disclosure of each of the specified features or components with or without the other.

The terms “comprising”, “having”, “including”, and “containing” are to be construed as open-ended terms (i.e., meaning “including, but not limited to”) unless otherwise noted. The term “consisting of” is to be construed as close-ended.

As used herein, “antibody” refers to a polypeptide whose amino acid sequence includes immunoglobulins and fragments thereof which specifically bind to a designated antigen, or fragments thereof. Antibodies in accordance with the present invention may be of any type (e.g., IgA, IgD, IgE, IgG, or IgM) or subtype (e.g., IgA1, IgA2, IgG1, IgG2, IgG3, or IgG4). Those of ordinary skill in the art will appreciate that a characteristic sequence or portion of an antibody may include amino acids found in one or more regions of an antibody (e.g., variable region, hypervariable region, constant region, heavy chain, light chain, and combinations thereof). Moreover, those of ordinary skill in the art will appreciate that a characteristic sequence or portion of an antibody may include one or more polypeptide chains, and may include sequence elements found in the same polypeptide chain or in different polypeptide chains.

As used herein, “antigen-binding fragment” refers to a portion of an antibody that retains the specificity of the binding characteristics of the parent antibody.

Antibodies or antigen-binding fragments thereof of the present disclosure may be isolated and/or substantially purified.

### Compounds, e.g., Immunoconjugates or Radioimmunoconjugates

In one aspect, this disclosure provides compounds, e.g., immunoconjugates or radioimmunoconjugates, comprising the following structure, or pharmaceutically acceptable salts thereof.

A-L¹-(L²)ₙ-B Formula I
wherein
A is a chelating moiety or a metal complex thereof,
B is an antibody or antigen-binding fragment thereof that is capable of binding to both EGFR and cMET, wherein the antibody or antigen-binding fragment thereof is featured as disclosed herein;
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
L² each independently has the structure of Formula II:

X¹-L³-Z¹ Formula II
wherein
   X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—*, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—*, or —NR¹—*; wherein “*” indicates the attachment point to L³, and R¹ is hydrogen, optionally substituted C₁-C₆ alkyl, optionally substituted C₁-C₆ heteroalkyl, or optionally substituted aryl or heteroaryl;
   L³ is optionally substituted C₁-C₅₀ alkyl or optionally substituted C₁-C₅₀ heteroalkyl; and
   Z¹ is —CH₂-#, —C(O)-#, —C(S)-#, —OC(O)-#, —C(O)O-#, —NR²C(O)-#, —C(O)NR²-#, or —NR²-#, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl.

Typical substituents of alkyl, heteroalkyl, aryl, or heteroaryl include, but are not limited to halo (e.g., F, Cl, Br, I), OH, CN, nitro, amino, C₁₋₆ alkyl, C₂₋₆ alkenyl, C₂₋₆ alkynyl, C₃₋₈ cycloalkyl, C₁₋₆ heteroalkyl, C₁₋₆ heterocycloalkyl, haloalkyl (e.g., CF₃), alkoxy (e.g., OCH₃), alkylamino (e.g., NH₂CH₃), sulfonyl, aryl, and heteroaryl.

In some embodiments, the compound (e.g., immunoconjugate or radioimmunoconjugate) has or comprises the structure shown below, or a metal complex thereof:

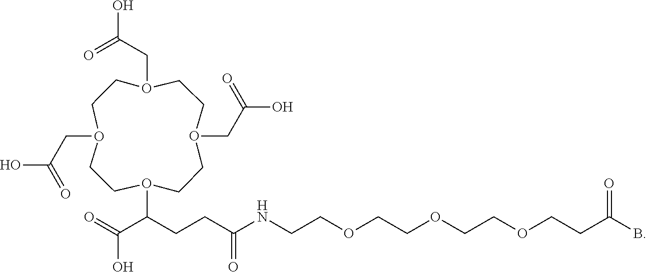

wherein B is an EGFR-cMET antibody or antigen-binding fragment thereof as disclosed herein.

In some embodiments, the compound of Formula I comprises one of the following structures, or a metal complex thereof:

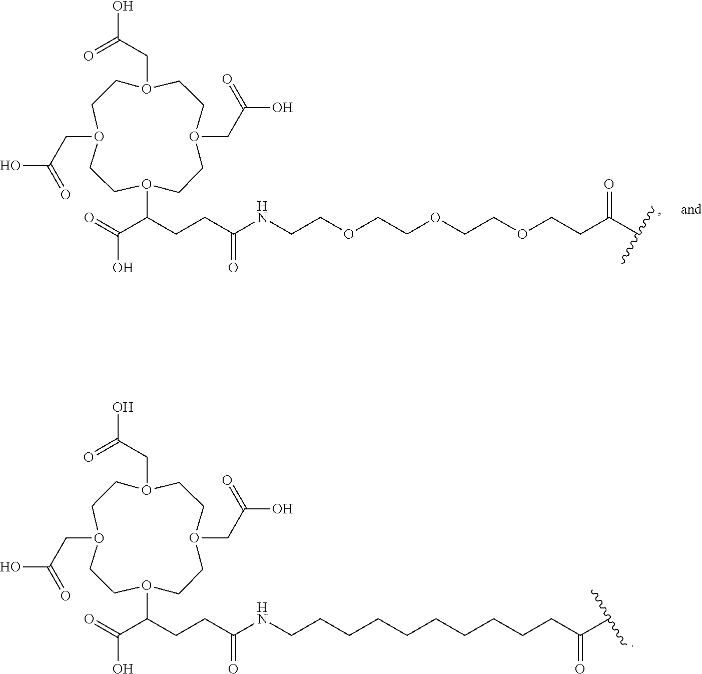

In some embodiments, provided compounds (e.g., immunoconjugates or radioimmunoconjugates) are capable of binding to different cell lines with varying expression levels of EGFR and cMET with a Kd value of at most about 25 nM, at most about 20 nM, at most about 15 nM, at most about 12.5 nM, at most about 10 nM, at most about 7.5 nM, at most about 7 nM, at most about 6.5 nM, at most about 6 nM, at most about 5 nM, at most about 4 nM, at most about 3.5 nM, at most about 3 nM, or at most about 2.5 nM. In some embodiments, provided compounds (e.g., immunoconjugates or radioimmunoconjugates) are capable of binding to different cell lines with varying expression levels of EGFR and cMET with a Kd value of about 15 nM, about 12.5 nM, about 10 nM, about 7.5 nM, about 7 nM, about 6.5 nM, about 6 nM, about 5 nM, about 4 mM, about 3.5 nM, about 3 nM, or about 2.5 nM.

In some embodiments, as further described herein, the compound (e.g., immunoconjugate or radioimmunoconjugate) comprises a chelating moiety or a metal complex thereof, which metal complex may comprise a radionuclide. In some such compounds, the average ratio or median ratio of the chelating moiety to the EGFR-cMET targeting moiety (e.g., EGFR-cMET antibody) is eight or less, seven or less, six or less, five or less, four or less, three or less, two or less, or about one. In some compounds, the average ratio or median ratio of the chelating moiety to the EGFR-cMET targeting moiety (e.g., EGFR-cMET antibody) is about one.

In some embodiments, after a radioimmunoconjugate is administered to a mammal, the proportion of radiation (of the total amount of radiation that is administered) that is excreted by the intestinal route, the renal route, or both is greater than the proportion of radiation excreted by a comparable mammal that has been administered a reference radioimmunoconjugate. By “reference immunoconjugate” it is meant a known radioimmunoconjugate that differs from a radioimmunoconjugate described herein at least by (1) having a different linker; (2) having a targeting moiety of a different size and/or (3) lacking a targeting moiety. In some embodiments, the reference radioimmunoconjugate is selected from the group consisting of [⁹⁰Y]-ibritumomab tiuxetan (Zevalin (⁹⁰Y)) and [¹¹¹In]-ibritumomab tiuxetan (Zevalin (¹¹¹In)).

In some embodiments, the proportion of radiation excreted by a given route or set of routes) is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater than the proportion of radiation excreted by the same route(s) by a comparable mammal that has been administered a reference radioimmunoconjugate. In some embodiments, the proportion of radiation excreted is at least 1.5-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, or at least 10-fold greater than proportion of radiation excreted by a comparable mammal that has been administered a reference radioimmunoconjugate. The extent of excretion can be measured by methods known in the art, e.g., by measuring radioactivity in urine and/or feces and/or by measuring total body radioactivity over a period time. See also, e.g., International Patent Publication WO 2018/024869.

In some embodiments, the extent of excretion is measured at a time period of at least or about 12 hours after administration, at least or about 24 hours after administration, at least or about 2 days after administration, at least or about 3 days after administration, at least or about 4 days after administration, at least or about 5 days after administration, at least or about 6 days after administration, or at least or about 7 days, after administration.

In some embodiments, after a compound (e.g., immunoconjugate or radioimmunoconjugate) has been administered to a mammal, the compound (e.g., immunoconjugate or radioimmunoconjugate) exhibits decreased off-target binding effects (e.g., toxicities) as compared to a reference compound (e.g., reference conjugate, e.g., a reference immunoconjugate such as a reference radioimmunoconjugate). In some embodiments, this decreased off-target binding effect is a feature of a compound (e.g., immunoconjugate or radioimmunoconjugate) that also exhibits a greater excretion rate as described herein.

### Targeting Moieties

Targeting moieties include any molecule or any part of a molecule that is capable of binding (e.g., capable of specifically binding, specifically binds to, etc.) to a given target, e.g., EGFR or cMET, or EGFR-cMET. In some embodiments, the targeting moiety comprises a protein or polypeptide. In some embodiments, the targeting moiety is selected from the group consisting of antibodies or antigen binding fragments thereof, nanobodies, affibodies, and consensus sequences from Fibronectin type III domains (e.g., Centyrins or Adnectins).

In some embodiments, a moiety is both a targeting and a therapeutic moiety, i.e., the moiety is capable of binding to a given target and also confers a therapeutic benefit.

In some embodiments, the targeting moiety has a molecular weight of at least 50 kDa, at least 75 kDa, at least 100 kDa, at least 125 kDa, at least 150 kDa, at least 175 kDa, at least 200 kDa, at least 225 kDa, at least 250 kDa, at least 275 kDa, or at least 300 kDa.

In some embodiments, the targeting moiety is capable of specifically binding to and inhibits both EGFR and cMET. By “inhibits,” it is meant that the targeting moiety at least partially inhibits one or more functions of EGFR, cMET, or both. In some embodiments, the targeting moiety impairs signaling downstream of EGFR, cMET, or both, e.g., results in the suppressed growth of tumor cells with varying expression levels of EGFR and cMET.

### Targets

### EGFR

Human EGFR (also known as proto-oncogene c-ErbB-1, receptor tyrosine-protein kinase erbB-1 and EC 2.7.10.1) is the protein identified by UniProt P00533. Alternative splicing of mRNA encoded by the human EGFR gene (also known as ERBB, ERBB1 and HER1) yields four isoforms: isoform 1 (UniProt: P00533-1, v2 (last sequence update: Nov. 1, 1997)); isoform 2 (UniProt: P00533-2, v1), which comprises the substitutions F404L and L405S relative to isoform 1, and which lacks the amino acid sequence corresponding to positions 406 to 1210 of isoform 1; isoform 3 (UniProt: P00533-3, v1), which comprises substitutions at position 628 to 705 of isoform 1, and which lacks the amino acid sequence corresponding to positions 706 to 1210 of isoform 1; and isoform 4 (UniProt: P00533-4), which comprises the substitution C628S relative to isoform 1, and which lacks the amino acid sequence corresponding to positions 629 to 1210 of isoform 1.

The structure and function of EGFR is reviewed e.g., in Ferguson, Annu Rev Biophys. (2008) 37: 353-373. EGFR is a transmembrane protein that is a receptor for members of the epidermal growth factor family (EGF family). The receptor comprises a large extracellular region, a single spanning transmembrane domain, an intracellular juxtamembrane domain, a tyrosine kinase domain and a C-terminal regulatory region. Binding of EGFR to a ligand induces receptor dimerization and autophosphorylation of several tyrosine residues (Y992, Y1045, Y1068, Y1148 and Y1173) in the C-terminal regulatory region of EGFR.

Aberrant EGFR expression/activity is implicated in many diseases, including nervous system disorders and many cancers.

In this specification “EGFR” refers to EGFR from any species and includes EGFR isoforms, fragments, variants or homologues from any species.

cMet

Human cMET (also known as Hepatocyte growth factor receptor (HGFR) or tyrosine-protein kinase Met) is the protein identified by UniProt P08581. Alternative splicing of mRNA encoded by the human MET gene yields three isoforms: isoform 1 (UniProt: P08581-1, v4 (last sequence update: Jul. 7, 2009)); isoform 2 (UniProt: P08581-2), in which the amino acid sequence “STWWKEPLNIVSFLFCFAS” is inserted at position 755 of isoform 1; and isoform 3 (UniProt: P08581-3) also known as Soluble met variant 4, in which the amino acid sequence corresponding to positions 755 to 764 of isoform 1 are substituted with “RHVNIALIQR” and which further lacks the amino acid sequence corresponding to positions 765 to 1390 of isoform 1.

The structure of cMET is reviewed e.g. in Gherardi, 2003, which is herein incorporated by reference in its entirety. cMET is a heterodimer made of an alpha chain (50 kDa) and a beta chain (145 kDa), which are disulphide linked. cMET comprises a N-terminal Sema domain, which mediates binding to hepatocyte growth factor (HGF) and an intracellular kinase domain. Ligand binding at the cell surface induces autophosphorylation of cMET on its intracellular domain that provides docking sites for downstream signalling molecules and the activation of several signalling cascades.

cMET is expressed in normal tissues on the surface of epithelial cells. cMET overexpression is observed in many human tumors and cancers, which is frequently associated with a metastatic phenotype and poor prognosis. Examples of cancers where high levels of cMET expression has been observed includes non-small cell lung cancer, pancreatic cancer, colorectal cancer, head and neck squamous cell carcinoma, breast cancer and esophageal-gastric cancer. In these cancers, co-expression of EGFR and cMET is often observed.

### Antibodies or Antigen-Binding Fragments Thereof

The term “antibody” describes an immunoglobulin whether natural or partly or wholly synthetically produced. The antibody may be human or humanised. The antibody is preferably a monoclonal antibody. Examples of antibodies are the immunoglobulin isotypes, such as immunoglobulin G (IgG), and their isotypic subclasses, such as IgG1, IgG2, IgG3 and IgG4, as well as fragments thereof.

The term “antibody”, as used herein, thus includes antibody fragments, as long as they display binding to the relevant target molecule(s). Examples of antibody fragments include Fv, scFv, Fab, scFab, F(ab′)₂, Fab₂, diabodies, triabodies, scFv-Fc, minibodies and single domain antibodies (e.g., VhH), etc.). Unless the context requires otherwise, the term “antibody”, as used herein, is thus equivalent to “antibody or fragment thereof”.

Antibodies and methods for their construction and use are well-known in the art and are described in, for example, Holliger & Hudson, Nature Biotechnology 23(9):1126-1136 (2005). It is possible to take monoclonal and other antibodies and use techniques of recombinant DNA technology to produce other antibody or chimeric molecules which retain the specificity of the original antibody. Such techniques may involve introducing CDRs or variable regions of one antibody into a different antibody (EP-A-184187, GB 2188638A and EP-A-239400).

In view of today's techniques in relation to monoclonal antibody technology, antibodies can be prepared to most antigens. The antigen-binding domain may be a part of an antibody (for example a Fab fragment) or a synthetic antibody fragment (for example a single chain Fv fragment (ScFv)). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimaeric antibodies are discussed by Neuberger, 1988.

Antibodies according to the present disclosure comprise an antigen-binding domain. An “antigen-binding domain” describes the part of a molecule that binds to all or part of the target antigen. Where an antigen is large, an antibody may only bind to a particular part of the antigen, which part is termed an epitope. An antibody antigen-binding site may be provided by one or more antibody variable domains. An antibody antigen-binding site preferably comprises a variable light (VL) region and variable heavy (VH) region. The VH and VL region of an antigen-binding domain together constitute the Fv region.

An antigen-binding domain generally comprises six complementarity-determining regions (CDRs); three in the VH region: HCDR1, HCDR2 and HCDR3, and three in the VL region: LCDR1, LCDR2, and LCDR3. The six CDRs together define the paratope of the antigen-binding domain, which is the part of the antigen-binding domain which binds to the target antigen.

The VH region and VL region comprise framework regions (FRs) either side of each CDR, which provide a scaffold for the CDRs. From N-terminus to C-terminus, VH regions comprise the following structure: N term-[HFR1]-[HCDR1]-[HFR2]-[HCDR2]-[HFR3]-[HCDR3]-[HFR4]-C term; and VL regions comprise the following structure: N term-[LFR1]-[LCDR1]-[LFR2]-[LCDR2]-[LFR3]-[LCDR3]-[LFR4]-C term.

There are several different conventions for defining antibody CDRs and FRs, such as those described in Kabat, 1991, Chothia, 1987, IMGT numbering as described in LeFranc, 2015, and VBASE2, as described in Retter, 2005. The CDRs and FRs of the VH regions and VL regions of the antibodies described herein were defined according to Kabat (Kabat, 1991).

Antibodies that comprise at least two antigen-binding domains, each of which being capable of binding to a different target may be termed “bispecific antibodies”. In contrast, antibodies that only bind a single target (e.g., EGFR or cMET) are termed “monospecific antibodies”. The present disclosure provides compounds, (e.g., radioimmunoconjugates) comprising a bispecific antibody or antigen-binding fragment thereof comprising a first antigen binding domain that is capable of binding EGFR and a second antigen-binding domain that is capable of binding cMET.

### Anti-EGFR Antigen-Binding Domains

The antigen-binding domain that is capable of binding EGFR comprises the CDRs of an antibody which is capable of binding to EGFR. In some embodiments, the antigen-binding domain is capable of binding EGFR additionally comprises the FRs of an antibody which is capable of binding to EGFR. That is, in some embodiments the antigen-binding domain that is capable of binding EGFR comprises the VH region and the VL region of an antibody which is capable of binding to EGFR.

In some embodiments the antigen-binding domain that is capable of binding EGFR comprises a VH region and a VL region which is, or which is derived from, the VH/VL region of an EGFR-binding antibody clone RAA22.

In some embodiments, the antigen-binding domain that is capable of binding EGFR comprises the three HCDRs or three LCDRs, preferably the three VH CDRs and the three VL CDRs, of anti-EGFR antibody clone RAA22. The VH and VL domain sequences of antibody RAA22 are described herein, and the three VH and three VL domain CDRs of said antibody may thus be determined from said sequences.

In some embodiments, the antigen-binding domain that is capable of binding EGFR comprises a VH region according to (1):
(1) a VH region comprising the following CDRs:
   HCDR1 having the amino acid sequence of SEQ ID NO: 1
   HCDR2 having the amino acid sequence of SEQ ID NO: 2
   HCDR3 having the amino acid sequence of SEQ ID NO: 3.

In some embodiments, the antigen-binding domain that is capable of binding EGFR comprises a VH region according to (1), wherein the VH region additionally comprises the FRs according to (2) below:
(2) HFR1 having the amino acid sequence of SEQ ID NO: 7
   HFR2 having the amino acid sequence of SEQ ID NO: 8
   HFR3 having the amino acid sequence of SEQ ID NO: 9
   HFR4 having the amino acid sequence of SEQ ID NO: 10,
   or a variant thereof in which one or two or three amino acids in one or more of HFR1, HFR2, HFR3, or HFR4 are substituted with another amino acid.

In some embodiments the antigen-binding domain that is capable of binding EGFR comprises a VH region according to (3) below:
(3) a VH region comprising the CDRs according to (1) and the FRs according to (2).

In some embodiments the antigen-binding domain that is capable of binding EGFR comprises a VH region according to (4):
(4) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 15.

In some embodiments the antigen-binding domain that is capable of binding EGFR comprises a VL region according to (5) below:
(5) a VL region comprising the following CDRs:
   LCDR1 having the amino acid sequence of SEQ ID NO: 4
   LCDR2 having the amino acid sequence of SEQ ID NO: 5
   LCDR3 having the amino acid sequence of SEQ ID NO: 6.

In some embodiments, the antigen-binding domain that is capable of binding EGFR comprises a VL region according to (5) above, wherein the VL region additionally comprises the FRs according to (6) below:
(6) LFR1 having the amino acid sequence of SEQ ID NO: 11
   LFR2 having the amino acid sequence of SEQ ID NO: 12
   LFR3 having the amino acid sequence of SEQ ID NO: 13
   LFR4 having the amino acid sequence of SEQ ID NO: 14,
   or a variant thereof in which one or two or three amino acids in one or more of LFR1, LFR2, LFR3, or LFR4 are substituted with another amino acid.

In some embodiments the antigen-binding domain that is capable of binding EGFR comprises a VL region comprising the CDRs according to (5) above, and the FRs according to (6) above.

In some embodiments the antigen-binding domain that is capable of binding EGFR comprises a VL region according to (7) below:
(7) a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 16.

In some embodiments the antigen-binding domain that is capable of binding EGFR comprises a VH region according to any one of (1) to (4) above, and a VL region according to any one of (5) to (7) above. In some embodiments, the antigen-binding domain comprises a VH region according to any one of (1), (3) and (4) and a VL region according to one of (5) and (7).

### Anti-cMET Antigen-Binding Domains

The antigen-binding domain that is capable of binding cMET comprises the CDRs of an antibody which is capable of binding to cMET. In some embodiments, the antigen-binding domain that is capable of binding cMET additionally comprises the FRs of an antibody which is capable of binding to cMET. That is, in some embodiments the antigen-binding domain that is capable of binding cMET comprises the VH region and the VL region of an antibody which is capable of binding to cMET.

In some embodiments the antigen-binding domain that is capable of binding cMET comprises a VH region and a VL region which is, or which is derived from, the VH/VL region of a cMET-binding antibody clone described herein (i.e. anti-cMET antibody clone B09-GL).

In some embodiments the antigen-binding domain that is capable of binding cMET comprises the three HCDRs or three LCDRs, preferably the three VH CDRs and the three VL CDRs, of cMET-binding antibody clone B09-GL. The VH and VL domain sequences of antibodies B09-GL are described herein, and the three VH and three VL domain CDRs of said antibodies may thus be determined from said sequences.

In some embodiments, the antigen-binding domain that is capable of binding cMET comprises a VH region according to (8) below:
(8) a VH region comprising the following CDRs:
   HCDR1 having the amino acid sequence of SEQ ID NO: 17
   HCDR2 having the amino acid sequence of SEQ ID NO: 18
   HCDR3 having the amino acid sequence of SEQ ID NO: 19.

In some embodiments, the antigen-binding domain that is capable of binding cMET comprises a VH region according to (8) above, wherein the VH region additionally comprises the FRs according to (9) below:
(9) HFR1 having the amino acid sequence of SEQ ID NO: 23
   HFR2 having the amino acid sequence of SEQ ID NO: 24
   HFR3 having the amino acid sequence of SEQ ID NO: 25
   HFR4 having the amino acid sequence of SEQ ID NO: 26,
   or a variant thereof in which one or two or three amino acids in one or more of HFR1, HFR2, HFR3, or HFR4 are substituted with another amino acid.

In some embodiments the antigen-binding domain that is capable of binding cMET comprises a VH according to (10) below:
(10) a VH region comprising the CDRs according to (8) and the FRs according to (9).

In some embodiments the antigen-binding domain that is capable of binding cMET comprises a VH region according to (11) below:
(11) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 31.

In some embodiments the antigen-binding domain that is capable of binding cMET comprises a VL region according to (12) below:
(12) a VL region comprising the following CDRs:
   LCDR1 having the amino acid sequence of SEQ ID NO: 20
   LCDR2 having the amino acid sequence of SEQ ID NO: 21
   LCDR3 having the amino acid sequence of SEQ ID NO: 22.

In some embodiments, the antigen-binding domain that is capable of binding cMET comprises a VL region according to (12) above, wherein the VL region additionally comprises the FRs according to (13) below:
(13) LFR1 having the amino acid sequence of SEQ ID NO: 27
   LFR2 having the amino acid sequence of SEQ ID NO: 28
   LFR3 having the amino acid sequence of SEQ ID NO: 29
   LFR4 having the amino acid sequence of SEQ ID NO: 30,
   or a variant thereof in which one or two or three amino acids in one or more of LFR1, LFR2, LFR3, or LFR4 are substituted with another amino acid.

In some embodiments the antigen-binding domain that is capable of binding cMET comprises a VL region according to (14) below:
(14) a VL region comprising the CDRs according to (12) and the FRs according to (13).

In some embodiments the antigen-binding domain that is capable of binding cMET comprises a VL region according to (15) below:
(15) a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 32.

In some embodiments the antigen-binding domain that is capable of binding cMET comprises a VH region according to any one of (8) to (11) above, and a VL region according to any one of (12) to (15) above.

### Antigen-Binding Domains of a Bispecific Antibody

The invention provides compounds (e.g., radioimmunoconjugates) comprising an antibody (i.e. a bispecific antibody) or antigen-binding fragment thereof conjugated to a radioactive payload, wherein the antibody or antigen-binding fragment thereof of the radioimmunoconjugate comprises a first antigen-binding domain that comprises the CDRs of an antigen-binding domain which is capable of binding to EGFR, and a second antigen-binding domain that comprises the CDRs of an antigen-binding domain which is capable of binding to cMET. In some embodiments, the first antigen-binding domain comprises the CDRs and the FRs of an antigen-binding domain which is capable of binding to EGFR and the second antigen-binding domain comprises the CDRs and the FRs of an antigen-binding domain which is capable of binding to cMET. That is, in some embodiments the antibody or antigen-binding fragment of the compound (e.g., radioimmunoconjugate) comprises a first antigen-binding domain comprising the VH region and the VL region of an antigen-binding domain which is capable of binding to EGFR and a second antigen-binding domain comprising the VH region and the VL region of an antigen-binding domain which is capable of binding to cMET.

In some embodiments, the first antigen-binding domain that is capable of binding EGFR comprises a VH region and a VL region which is, or which is derived from, the VH/VL region of the EGFR-binding antibody clone RAA22, and the second antigen-binding domain that is capable of binding cMET comprises a VH region and a VL region which is, or which is derived from, the VH/VL region of the cMET-binding antibody clone B09-GL. Such a bispecific antibody may be termed “RAA22/B09” or “RAA22/B09 bispecific antibody”.

In some embodiments the first antigen-binding domain comprises:
a VH region according to any one of (1) to (4) above and a VL region according to any one of (5) to (7) above; and
   the second antigen-binding domain comprises:
a VH region according to any one of (8) to (11) above, and a VL region according to any one of (12) to (15) above.

In certain embodiments, the compound (e.g., radioimmunoconjugate) of this invention comprises an antibody or antigen-binding fragment thereof comprising:
a first antigen-binding domain that is capable of binding epidermal growth factor receptor (EGFR); and
a second antigen-binding domain that is capable of binding cMET,
   wherein the first antigen-binding domain comprises:
(i) a heavy chain variable (VH) region comprising the following complementarity determining regions (CDRs):
   HCDR1 having the amino acid sequence of SEQ ID NO: 1
   HCDR2 having the amino acid sequence of SEQ ID NO: 2
   HCDR3 having the amino acid sequence of SEQ ID NO: 3; and
(ii) a light chain variable (VL) region comprising the following CDRs:
   LCDR1 having the amino acid sequence of SEQ ID NO: 4
   LCDR2 having the amino acid sequence of SEQ ID NO: 5
   LCDR3 having the amino acid sequence of SEQ ID NO: 6.

In certain embodiments, the first antigen-binding domain comprises:
a VH region comprising an amino acid sequence having at least 70% sequence identity to the amino acid sequence of SEQ ID NO: 15; and
a VL region comprising an amino acid sequence having at least 70% sequence identity to the amino acid sequence of SEQ ID NO: 16,
   and/or the second antigen-binding domain comprises:
a VH region comprising an amino acid sequence having at least 70% sequence identity to the amino acid sequence of SEQ ID NO: 31; and a VL region comprising an amino acid sequence having at least 70% sequence identity to the amino acid sequence of SEQ ID NO: 32.

### Constant Region

In some embodiments the antibody or antigen-binding fragment thereof of the compound (e.g., radioimmunoconjugate) described herein comprises an immunoglobulin heavy chain constant (CH) region. In some embodiments the CH is, or is derived from, the heavy chain constant sequence of an IgG (e.g., IgG1, IgG2, IgG3, IgG4), IgA (e.g., IgA1, IgA2), IgD, IgE or IgM.

In some embodiments the CH region is human immunoglobulin G1 constant (IGHG1; UniProt: P01857-1, v1; SEQ ID NO: 33) or a fragment thereof.

In some embodiments, the CH region comprises an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 39 or 40.

In some embodiments, the antibody or antigen-binding fragment thereof of the compound (e.g., radioimmunoconjugate) comprises a heavy chain that comprises or consists of a VH region as described herein and a CH region as described herein.

In some embodiments, the antibody or antigen-binding fragment thereof of the compound (e.g., radioimmunoconjugate) described herein comprises an immunoglobulin light chain constant (CL) region or a fragment thereof. In some embodiments, the CL region is, or is derived from a kappa CL region set forth in SEQ ID NO: 34. In some embodiments, the CL region is, or is derived from a lambda CL region set forth in SEQ ID NO: 41. In some embodiments, the antibody or antigen-binding fragment thereof of the compound (e.g., radioimmunoconjugate) comprises: a first CL region that is, or is derived from, a kappa CL region set forth in SEQ ID NO: 34; and a second CL region that is, or is derived from, a lambda CL region set forth in SEQ ID NO: 41.

In some embodiments, the antibody or antigen-binding fragment thereof of the compound (e.g., radioimmunoconjugate) described herein comprises:
a first heavy chain, wherein the first heavy chain comprises the VH region of the first antigen-binding domain, and a first heavy chain constant (CH) region or a fragment thereof,
a first light chain, wherein the first light chain comprises the VL region of the first antigen-binding domain, and a first light chain constant (CL) region or a fragment thereof;
a second heavy chain, wherein the second heavy chain comprises the VH region of the second antigen-binding domain, and a second heavy chain constant (CH) region or a fragment thereof; and
a second light chain, wherein the second light chain comprises the VL region of the second antigen-binding domain, and a second light chain constant (CL) region or a fragment thereof.

It will be understood that when an antibody or antigen-binding fragment thereof comprises a first VH region and a first CH region, that these regions together form a first heavy chain of the antibody or antigen-binding fragment thereof, that is that the first VH and first CH regions are connected to each other. Similarly, a second VH region and a second CH region forms a second heavy chain of the antibody or antigen-binding fragment thereof, a first VL region and a first CL region form a first light chain of the antibody or antigen-binding fragment thereof, and a second VL region and a second CL region form a second light chain of the antibody or antigen-binding fragment thereof.

In some embodiments, the antibody or antigen-binding fragment thereof of the compound (e.g., radioimmunoconjugate) comprises a first and second heavy chain, wherein
(i) the first heavy chain comprises an amino acid sequence which has at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of the heavy chain set forth in SEQ ID NO: 35; and
(ii) the second heavy chain comprises an amino acid sequence which has at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of the heavy chain set forth in SEQ ID NO: 36.

In some embodiments, the antibody or antigen-binding fragment thereof of the compound (e.g., radioimmunoconjugate) described herein comprises a light chain that comprises or consists of a VL region as described herein and a CL region as described herein.

In some embodiments, the antibody or antigen-binding fragment thereof of the compound (e.g., radioimmunoconjugate) described herein comprises a first and second light chain, wherein
(i) the first light chain comprises an amino acid sequence which has at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of the light chain set forth in SEQ ID NO: 37; and
(ii) the second light chain comprises an amino acid sequence which has at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of the light chain set forth in SEQ ID NO: 38.

The CH, CL, heavy chain and/or light chain of the antibody or antigen-binding fragment thereof of the compounds (e.g., radioimmunoconjugates) described herein may comprise one or more modifications, for example to abrogate or reduce Fc effector functions, promote formation of a heterodimeric antibody or antigen-binding fragment thereof, to increase the efficacy of cognate heavy and light chain pairing, and/or to assist with conjugate formation as described in more detail below. A CH, CL, heavy chain and light chain that has been modified may be referred to as a modified CH, CL, heavy chain and light chain, respectively.

The antibody or antigen-binding fragment thereof of the compound (e.g., radioimmunoconjugate) may comprise a mutation in the CH region(s) of the heavy chain(s) to reduce or abrogate binding of the antibody or antigen-binding fragment thereof to one or more Fcγ receptors, such as FcγRI, FcγRIIa, FcγRIIb, FcγRIII and/or to complement. Such mutations abrogate or reduce Fc effector functions. Mutations for reduce or abrogate binding of an antibody to one or more Fcγ receptors and complement are known and include the “triple mutation” or “TM” of L234F/L235E/P331S described for example in Organesyan, 2008. Other mutations that are known to modulate antibody effector function are described for example in Wang, 2018.

Examples of CH regions comprising the triple mutation are SEQ ID NOs: 39 and 40. Thus, in some embodiments, one of the first and second heavy chains comprises a CH region having an amino acid sequence having at least 70%, at least 80%, at least 90%, at least 95%, or 100% sequence identity to the sequence set forth in SEQ ID NO: 39 and the other heavy chain comprises a CH region having an amino acid sequence having at least 70%, at least 80%, at least 90%, at least 95%, or 100% sequence identity to the sequence set forth in SEQ ID NO: 40, wherein one or both (preferably both) of the CH regions comprise a phenylalanine at position 234, glutamic acid at position 235, and serine at position 331, wherein the numbering is as per the EU index.

Examples of heavy chains of the disclosure comprising a CH region containing the triple mutation are SEQ ID NOs: 35 and 36. Thus, in some embodiments, one of the first and second heavy chains has an amino acid sequence having at least 70%, at least 80%, at least 90%, at least 95%, or 100% sequence identity to the sequence set forth in SEQ ID NO: 35 and the other heavy chain has an amino acid sequence having at least 70%, at least 80%, at least 90%, at least 95%, or 100% sequence identity to the sequence set forth in SEQ ID NO: 36, wherein one or both (preferably both) of the have chains comprise a phenylalanine at position 234, glutamic acid at position 235, and serine at position 331, wherein the numbering is as per the EU index.

The VL and CL region, and the VH region and CH1 region of an antibody or antigen-binding fragment thereof of the compound (e.g., radioimmunoconjugate) together constitute the Fab region. The remainder of the antibody or antigen-binding fragment thereof of the compound (e.g., radioimmunoconjugate) constitute the Fc region.

Unless otherwise specified, amino acid residue positions in the constant domain, including the position of amino acid sequences, substitutions, deletions and insertions as described herein, are numbered according to EU numbering (Edelman, 2007).

### Bispecificformats

The disclosure provides asymmetrical IgG-like bispecific antibodies or antigen-binding fragments thereof within the compound (e.g., radioimmunoconjugates) disclosed herein. Asymmetrical bispecific molecules are typically monovalent for each target. As described in, for example, Klein, 2012, the concept of monovalent bispecific IgG is thought to have a unique therapeutic niche in that they (i) do not cause receptor homodimerization, (ii) potentially have reduced toxicity on non-target tissues due to loss of avidity for each antigen, and (iii) have better selectivity when both antigens are either selectively restricted or abundantly expressed on target cells. Thus, in some embodiments, the antibody or antigen-binding fragment thereof is an asymmetrical IgG-like bispecific antibody or antigen-binding fragment thereof.

Asymmetrical IgG-like bispecific antibodies involve heterodimerization of two distinct heavy chain and correct pairing of the cognate light chain and heavy chain. Heterodimerization of the heavy chains can be addressed by several techniques, such as knobs-into-holes, electrostatic steering of CH3, CH3 strand exchanged engineered domains and leucine zippers. The pairing of the correct light and heavy chain can be ensured by using one of these heavy chain heterodimerization techniques along with the use of a common light chain, domain cross-over between CH1 and CL, coupling of the heavy and light chains with a linker, in vitro assembly of heavy chain-light chain dimers from two separate monoclonals, interface engineering of an entire Fab domain, or disulfide engineering of the CH1/CL interface.

A particular exemplified format of asymmetrical IgG-like bispecific antibodies is referred to as “DuetMab”. DuetMab antibodies uses KIH technology for heterodimerization of 2 distinct heavy chains and increases the efficacy of cognate heavy and light chain pairing by replacing the native disulphide bond in one of the CH1-CL interfaces with an engineered disulphide bond. Disclosure related to DuetMab can found e.g., in U.S. Pat. No. 9,527,927 and Mazor, 2015, which are herein incorporated by reference in their entirety.

In some embodiments, the antibody or antigen-binding fragment thereof of the compound (e.g., radioimmunoconjugate) comprises:
(a) a modified CH region, wherein the modified heavy chain comprises a substitution of a native non-cysteine amino acid to a cysteine amino acid; and
(b) a modified corresponding CL region, wherein the modified CL comprises a substitution of a native non-cysteine amino acid to a cysteine amino acid,
wherein either:
(i) the first heavy chain comprises the modified CH region and the first light chain comprises the modified corresponding CL region; or
(ii) the second heavy chain comprises the modified CH region and the second light chain comprises the modified corresponding CL region.

In some embodiments, the substituted cysteine of the modified CH region, resulting from the substitution of the native non-cysteine amino acid to the cysteine amino acid, and the substituted cysteine of the modified corresponding CL region, resulting from the substitution of the native non-cysteine amino acid to the cysteine amino acid, can form a disulphide bond.

In some embodiments, the modified CH region comprises a substitution of a native non-cysteine amino acid to a cysteine amino acid at position 126; and the modified corresponding CL region comprises a substitution of a native non-cysteine amino acid to a cysteine at position 121, wherein the numbering of the constant region is as per the EU index.

In some embodiments, the modified CH region comprises a substitution of a native non-cysteine amino acid to a cysteine amino acid at position 126 and a substitution of a native cysteine amino acid to a non-cysteine amino acid at position 219, for example to a valine; and the modified corresponding CL region comprises a substitution of a native non-cysteine amino acid to a cysteine at position 121 and a substitution of a native cysteine amino acid to a non-cysteine amino acid at position 214, for example to a valine, where the numbering of the constant region is as per the EU index.

In some embodiments, the antibody or antigen-binding fragment thereof comprises a second CH region and a second corresponding light chain, wherein the second CH region and second corresponding CL do not comprise a substitution of a native non-cysteine amino acid to a cysteine amino acid and do not comprise a substitution of a native cysteine to a non-cysteine amino acid.

### Functional Properties of the Antibodies or Antigen-Binding Fragments Thereof of the Compounds (e.g., Radioimmunoconjugates)

### Binding Affinity

The antibodies or antigen-binding fragments thereof of the radioimmunoconjugates described herein may be characterized by the antigen-binding domain that is capable of binding EGFR having a particular affinity for EGFR and/or the antigen-binding domain that is capable of binding cMET having a particular affinity for cMET. The binding affinity of an antibody or antigen-binding fragment thereof of the compound (e.g., radioimmunoconjugate) to a cognate antigen, such as human, mouse or cynomolgus EGFR or cMET can be determined by surface plasmon resonance (SPR), using Biacore, for example. The binding affinity can be determined using an antibody or antigen-binding fragment thereof, for example as part of a bispecific antibody that comprises a first antigen-binding domain that is capable of binding EGFR and a second antigen-binding domain that is capable of binding cMET. Alternatively, the binding affinity can be determined using an antibody or antigen-binding fragment thereof that is monospecific for EGFR or cMET. In some embodiments, the binding affinity is determined using BIACore as provided herein or known in the field.

Binding affinity is typically measured by Kd (the equilibrium dissociation constant between the antigen-binding domain and its antigen). As is well understood, the lower the Kd value, the higher the binding affinity of the antigen-binding domain. For example, an antigen-binding domain that is capable of binding to a target with a Kd of 10 nM would be considered to be binding said target with a higher affinity than an antigen-binding domain that is capable of binding to the same target with a Kd of 100 nM.

Reference to human EGFR may refer to a polypeptide comprising the extracellular domain of EGFR, such as one having the amino acid sequence set forth in SEQ ID NO: 42. Reference to mouse EGFR may refer to a polypeptide produced from the molecule available from SinoBiological with catalogue #51091-M08H. Reference to cynomolgus EGFR may refer to the amino acid sequence set forth in SEQ ID NO: 46. Reference to human cMET may refer to a polypeptide having the amino acid sequence set forth in SEQ ID NO: 43. Reference to mouse cMET may refer to a polypeptide produced from the molecule available from SinoBiological with catalogue #50622-M08H. Reference to cynomolgus cMET may refer to the amino acid sequence set forth in SEQ ID NO: 44.

### EGFR Affinity

The antibodies or antigen-binding fragments thereof of the compounds (e.g., radioimmunoconjugates) described herein may comprise an antigen-binding domain that is capable of binding EGFR with a low affinity. As used herein, “low affinity” refers to a first antigen-binding domain that is capable of binding human EGFR with a dissociation constant (Kd) that is equal to or higher than 10 nM. Antibodies or antigen-binding fragments of the compounds (e.g., radioimmunoconjugates) described herein comprising such a low affinity EGFR antigen-binding domain may display reduced on-target toxicity in normal tissues such as skin toxicity and therefore have an improved safety profile compared to conjugates comprising an EGFR antigen-binding domain that is capable of binding human EGFR with a “higher affinity”. As used herein, “higher affinity” or “high affinity” refers to a first antigen binding domain that is capable of binding human EGFR with a Kd that is lower than 10 nM.

The antigen-binding domain that is capable of binding EGFR may bind to human EGFR with an affinity having a Kd equal to or higher than 10 nM, 15 nM, 20 nM, 25 nM, 30 nM, 35 nM, or 40 nM. Alternatively, the antigen-binding domain that is capable of binding EGFR may bind to human EGFR with a Kd of between 10 and 100 nM, between 20 and 100 nM, between 30 and 100 nM, between 40 and 100 nM, between 10 and 80 nM, between 20 and 80 nM, between 30 and 80 nM, between 40 and 80 nM, between, between 10 and 70 nM, between 20 and 70 nM, between 30 and 70 nM, between 40 and 70 nM, between 10 and 60 nM, between 20 and 60 nM, between 30 and 60 nM, between 40 and 60 nM, between 10 and 50 nM, between 20 and 50 nM, between 30 and 50 nM, or between 40 and 50 nM.

The antigen-binding domain that is capable of binding EGFR may bind to human EGFR with an affinity that is lower than the affinity of an antigen-binding domain comprising the variable heavy (VH) region sequence and variable light (VL) region sequence of antibody QD6, as set forth in SEQ ID NOs: 47 and 48, respectively.

For example, the antigen-binding domain that is capable of binding EGFR may bind to human EGFR with an affinity having a Kd that is at least 2-fold, 3-fold, 4-fold, 5-fold, 6-fold, or 7-fold higher than the Kd at which an antigen-binding domain comprising the heavy chain sequence and light chain sequence of antibody QD6 set forth in SEQ ID NOs: 47 and 48, respectively, binds human EGFR. Alternatively, the antigen-binding domain that is capable of binding EGFR may bind to human EGFR with an affinity having a Kd that is between 2- and 10-fold higher, between 3- and 10-fold higher, between 4- and 10-fold higher, between 5- and 10-fold higher, between 6- and 10-fold higher, between 7- and 10-fold higher, between 2- and 9-fold higher, between 3- and 9-fold higher, between 4- and 9-fold higher, between 5- and 9-fold higher, between 6- and 9-fold higher, between 7- and 9-fold higher, between 2- and 8-fold higher, between 3- and 8-fold higher, between 4- and 8-fold higher, between 5- and 8-fold higher, between 6- and 8-fold higher, between 7- and 8-fold higher than the Kd at which an antigen-binding domain comprising the heavy chain sequence and light chain sequence of antibody QD6 set forth in SEQ ID NOs: 47 and 48, respectively, binds human EGFR.

The antigen-binding domain that is capable of binding EGFR may bind to human EGFR with an affinity that is similar to the affinity that an antigen-binding domain comprising the variable heavy region sequence and variable light region sequence of antibody RAA22 set forth in SEQ ID NOs: 15 and 16, respectively. For example, the antigen-binding domain that is capable of binding EGFR may bind to human EGFR with an affinity having a Kd that is less than 5-fold different, less than 4-fold different, less than 3-fold different, less than 2-fold different, less than 1-fold different or less than 0.5-fold different than the Kd at which an antigen-binding domain comprising the variable heavy region sequence and variable light region sequence of antibody RAA22 set forth in SEQ ID NOs: 15 and 16, respectively, binds human EGFR.

The antigen-binding domain that is capable of binding EGFR may also be capable of binding cynomolgus EGFR. For example, the antigen-binding domain that is capable of binding EGFR may bind to cynomolgus EGFR with an affinity having a Kd that is less than 700 nM, less than 600 nM, less than 500 nM, less than 400 nM, less than 300 nM, or less than 250 nM. Alternatively, the antigen-binding domain that is capable of binding EGFR may bind to cynomolgus EGFR with an affinity having a Kd of between 100 and 700 nM, between 100 and 600 nM, between 100 and 500 nM, between 100 and 400 nM, between 100 and 300 nM, between 150 and 250 nM, between 100 and 200 nM. The antigen-binding domain that is capable of binding EGFR may bind to cynomolgus EGFR with a Kd that is less than or equal 10-, 9-, 8-, 7-, 6-, 5-, 4-, 3-fold higher Kd than the antigen-binding domain binds to human EGFR.

The antigen-binding domain is capable of binding EGFR may also be capable of binding mouse EGFR. For example, the antigen-binding domain that is capable of binding EGFR may bind to mouse EGFR with an affinity having a Kd that is less than 1 μM, less than 900 nM, less than 800 nM, less than 700 nM, less than 600 nM or less than 650 nM.

Alternatively, the antigen-binding domain that is capable of binding EGFR may bind to mouse EGFR with a Kd of between 100 nM and 1 μM, between 200 and 900 nM, between 300 and 800 nM, between 400 and 700 nM, between 400 and 600 nM, or between 450 and 550 nM.

Preferably, the antigen-binding domain that is capable of binding EGFR is capable of binding human EGFR and cynomolgus EGFR. This cross-reactivity is advantageous, as it allows dosing and safety testing of the antibodies and conjugates to be performed in cynomolgus monkeys during preclinical development. Even more preferably, the antigen-binding domain that is capable of binding EGFR is capable of binding human EGFR, cynomolgus EGFR and mouse EGFR. For example, the antigen-binding domain that is capable of binding EGFR may be capable of binding human EGFR, cynomolgus EGFR and mouse EGFR with the Kd values set out above (e.g. human EGFR with a Kd of between 10 and 100 nM, cynomolgus EGFR with a Kd of between 100 and 700 nM and mouse EGFR with a Kd of between 100 nM and 1 μM).

cMET Affinity

The antigen-binding domain that is capable of binding cMET may bind to human cMET with an affinity having a Kd of lower than 20 nM, 15 nM, 12 nM, 11 nM, 10 nM, 9 nM, 8 nM, 7 nM, 6 nM, 5 nM, 4 nM, 3 nM or 2.5 nM. Alternatively, antigen-binding domain that is capable of binding cMET may bind to human cMET with an affinity having a Kd of between 1 and 20 nM, between 1 and 15 nM, between 1 and 10 nM, between 1 and 9 nM, between 1 and 8 nM, between 1 and 7 nM, between 1 and 6 nM, between 1 and 5 nM, between 1 and 4 nM, between 1 and 3 nM, between 1 and 2.5 nM, or between 2 and 2.5 nM.

The antigen-binding domain that is capable of binding cMET may be capable of binding cynomolgus cMET. For example, the antigen-binding domain that may be capable of binding cMET may bind to cynomolgus cMET with an affinity having a Kd that is lower than 30 nM, 25 nM, 20 nM, 15 nM, 10 nM, 9 nM, 8 nM, 7 nM, 6 nM, 5 nM, 4 nM, 3 nM, or 2.5 nM. Alternatively, the antigen-binding domain that is capable of binding cMET may bind to cynomolgus cMET with an affinity having a Kd of between 1 and 20 nM, between 1 and 15 nM, between 1 and 10 nM, between 1 and 9 nM, between 1 and 8 nM, between 1 and 7 nM, between 1 and 6 nM, between 1 and 5 nM, between 1 and 4 nM, between 1 and 3 nM, between 1 and 2.5 nM, or between 2 and 2.5 nM. The antigen-binding domain that is capable of binding cMET may bind to cynomolgus cMET with an affinity having a Kd that is less than or equal 10-, 9-, 8-, 7-, 6-, 5-, 4-, 3- , 2-, 1-fold higher Kd than the antigen-binding domain binds to human cMET.

Preferably, the antigen-binding domain that is capable of binding cMET is capable of binding human cMET and cynomolgus cMET. This cross-reactivity is advantageous, as it allows dosing and safety testing of the antibodies to be performed in cynomolgus monkeys during preclinical development. For example, the antigen-binding domain that is capable of binding cMET may be capable of binding human cMET and cynomolgus cMET with the Kd values set out above (e.g., human cMET with a Kd of between 1 and 20 nM and cynomolgus cMET with a Kd of between 1 and 20 nM).

### Specific Binding

The antibodies or antigen-binding fragments thereof of the compounds (e.g., radioimmunoconjugates) described herein may comprise an antigen-binding domain that is capable of specifically binding EGFR. The antibodies or antigen-binding fragments of the compounds (e.g., radioimmunoconjugates) described herein may comprise an antigen-binding domain that is capable of specifically binding cMET. The antibodies or antigen-binding fragments of the compounds (e.g., radioimmunoconjugates) described herein may comprise a first antigen-binding domain that is capable of binding EGFR, which is a first antigen-binding domain that is capable of specifically binding EGFR, and a second antigen-binding domain that is capable of binding cMET, which is a first antigen-binding domain that is capable of specifically binding cMET.

The term “specific” may refer to the situation in which the antigen-binding domain will not show any significant binding to molecules other than its specific binding partner(s), here, EGFR or cMET. Such molecules are referred to as “non-target molecules”. The term “specific” is also applicable where the antibody or antigen-binding fragment thereof is specific for particular epitopes, such as epitopes on EGFR or cMET, that are carried by a number of antigens in which case the antibody or antigen-binding fragment thereof will be able to bind to the various antigens carrying the epitope.

In some embodiments, an antibody or antigen-binding fragment thereof of the compound (e.g., radioimmunoconjugate) is considered to not show any significant binding to a non-target molecule if the extent of binding to a non-target molecule is less than about 10% of the binding of the antibody or antigen-binding fragment thereof to the target as measured, e.g., by ELISA, SPR, Bio-Layer Interferometry (BLI), MicroScale Thermophoresis (MST), or by a radioimmunoassay (RIA). Alternatively, the binding specificity may be reflected in terms of binding affinity, where the antibodies or antigen-binding fragments thereof of the compounds (e.g., radioimmunoconjugates) described herein are capable of binding to EGFR and/or cMET with an affinity that is at least 0.1 order of magnitude greater than the affinity towards another, non-target molecule. In some embodiments, the antibody or antigen-binding fragment thereof of the compound (e.g., radioimmunoconjugate) of the present disclosure is capable of binding to EGFR and/or cMET with an affinity that is one of at least 0.2, 0.3, 0.4, 0.5, 0.6, 0.7, 0.8, 0.9, 1.0, 1.5, or 2.0 orders of magnitude greater than the affinity towards another, non-target molecule.

EGFR is a member of the ErbB family of receptors, a subfamily of four closely related receptor tyrosine kinases: EGFR, HER2, HER3 and HER4. The RAA22 antigen-binding domain showed no binding to HER2, HER3 and HER4, demonstrating that this antigen-binding domain binds EGFR specifically. Thus, in certain embodiments, the antigen-binding domain that is capable of binding EGFR does not bind, or does not show any significant binding, to HER2, HER3 or HER4.

cMET is a member of the subfamily of receptor tyrosine kinases that includes Ron and Sema 4a. The B09-GL antigen-binding domain showed no binding to Ron and Sema 4a, demonstrating that this antigen-binding domain binds cMET specifically. Thus, in certain embodiments, the antigen-binding domain that is capable of binding cMET does not bind, or does not show any significant binding, to Ron, Sema 4a.

### Concurrent Engagement

The antibodies or antigen-binding fragments thereof of the compounds (e.g., radioimmunoconjugates) in the present disclosure comprising a first antigen-binding domain that is capable of binding EGFR and a second antigen-binding domain that is capable of binding cMET described herein may be characterized by the ability of both the antigen-binding domains to concurrently engage their respective EGFR and cMET targets. Antibodies or antigen-binding fragments thereof of the presently disclosed compounds (e.g., radioimmunoconjugates) with the ability to concurrently engage EGFR and cMET are expected to be advantageous, as numerous tumors are known to co-express both EGFR and cMET and therefore can be targeted by compounds of the disclosure. Thus, in some embodiments, the antibody or antigen-binding fragment thereof of the presently disclosed compounds (e.g., radioimmunoconjugates) is able to concurrently engage EGFR and cMET.

Further details of these methods to measure concurrent engagement can be found in the examples.

### Antibody (or Antigen-Binding Fragment Thereof) Internalisation

The antibodies or antigen-binding fragments thereof of the compounds (e.g., radioimmunoconjugates) described herein may be characterised by their ability to mediate efficient internalisation.

Internalisation of an antibody or antigen-binding fragment thereof (or compound (e.g., radioimmunoconjugate) comprising said antibody or antigen-binding fragment thereof) by cells can be analysed by contacting live cells with the antibody or antigen-binding fragment thereof, and detecting the antibody (or antigen-binding fragment thereof) or compound (e.g., radioimmunoconjugate) after sufficient period of time for internalisation. Internalisation can be determined by detection of the localisation of the antibody (or antigen-binding fragment thereof) or compound (e.g., radioimmunoconjugate). Where the antibody (or antigen-binding fragment thereof) or compound (e.g., radioimmunoconjugate) remains on the surface of the cell (e.g., is detected on the cell surface, and/or is not detected inside the cell), the antibody (or antigen-binding fragment thereof) or compound (e.g., radioimmunoconjugate) is determined not to have been internalised. Where the antibody (or antigen-binding fragment thereof) or compound (e.g., radioimmunoconjugate) is detected inside the cell (e.g., localised to the cytoplasm or a cellular organelle), the antibody (or antigen-binding fragment thereof) or compound (e.g., radioimmunoconjugate) is determined to have been internalised.

An exemplary method for visualising whether the antibody (or antigen-binding fragment thereof) or compound (e.g., radioimmunoconjugate) is able to mediate efficient internalisation involves labelling the antibody with pH sensitive dyes that exhibit fluorescent at an acidic pH and adding these labelled antibodies or conjugates to cells. Internalisation into the cell can be detected by monitoring fluorescence. The antibody (or antigen-binding fragment thereof) or compound (e.g., radioimmunoconjugate) is considered able to mediate internalisation and delivery to lysosomes if the fluorescence observed is greater than that of a labelled non-binding control antibody (or antigen-binding fragment thereof) or compound (e.g., radioimmunoconjugate) over a certain time period, for example 48 hours. Further details of this method to visualise antibody (or antigen-binding fragment thereof) internalisation can be found in the examples.

The antibodies or antigen-binding fragments thereof of the compounds (e.g., radioimmunoconjugates) comprising a first antigen-binding domain that is capable of binding EGFR and a second antigen-binding domain that is capable of binding cMET may be characterised by their ability to mediate more efficient internalisation when compared to the EGFR or cMET monospecific controls. Antibodies or antigen-binding fragments thereof of compounds (e.g., radioimmunoconjugates) that exhibit this property are expected to be advantageous, as they are expected to display greater selectivity to tumor cells co-expressing both targets and could minimise the impact of the antibody or antigen-binding fragment thereof in normal tissues that do not display significant levels of co-expression.

In some embodiments, provided herein are compounds (e.g., immunoconjugates or radioimmunoconjugates) comprising antibodies or antigen-binding fragments that comprise a heavy chain variable domain and a light chain variable domain, wherein the heavy chain variable domain comprises a CDR-H1, CDR-H2, and CDR-H3, and the light chain variable domain comprises a CDR-L1, CDR-L2, and CDR-L3, wherein the CDR-H1, CDR-H2, CDR-H3, CDR-L1, CDR-L2, and CDR-L3, are those of an antibody described in the Sequence Annex provided below.

In some embodiments, provided are compounds (e.g., immunoconjugates or radioimmunoconjugates) comprising antibodies or antigen-binding fragments that are variants of the antibodies shown in Sequence Annex, in that such antibodies or antigen-binding fragments have CDR sequences that differ by no more than three amino acid residues (e.g., three or two or one amino acid residue(s)) per CDR from the CDR sequences of an antibody described in Sequence Annex. In some embodiments, provided are compounds (e.g., immunoconjugates or radioimmunoconjugates) comprising antibodies or antigen-binding fragments that are variants of the antibodies shown in Sequence Annex, in that such antibodies or antigen-binding fragments have a set of six CDRs whose sequences collectively differ by no more than three amino acid residues (e.g., three or two or one amino acid residues) from the CDRs of an antibody described in Sequence Annex.

In some embodiments, provided are compounds (e.g., immunoconjugates or radioimmunoconjugates) comprising antibodies or antigen-binding fragments that comprise a heavy chain variable domain and a light chain variable domain which comprise heavy chain variable domain and light chain variable sequences of an antibody described in Sequence Annex. In some embodiments, provided are compounds (e.g., immunoconjugates or radioimmunoconjugates) comprising antibodies or antigen-binding fragments that are variants of the antibodies shown in Sequence Annex, in that such antibodies or antigen-binding fragments have (1) a heavy chain domain comprising an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of the heavy chain variable domain of an antibody described in Sequence Annex; and (2) a light chain domain comprising an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of a light chain variable domain of the same antibody in Sequence Annex.

### Sequence Annex

### CDR Sequences for Anti-EGFR Antibody Clone RAA22

HCDR1-
(SEQ ID NO: 1)
DNDFS
HCDR2-
(SEQ ID NO: 2)
AIVAVFRTETYAQKFQD
HCDR3-
(SEQ ID NO: 3)
RLMSAISGPGAPLLM
LCDR1-
(SEQ ID NO: 4)
TGTSSDVGGYNYVS
LCDR2-
(SEQ ID NO: 5)
DVSKRPS
LCDR3-
(SEQ ID NO: 6)
SSYTSSDTLEI

### FR Sequences for Anti-EGFR Antibody Clones RAA22

HFR1-
(SEQ ID NO: 7)
QVQLVQSGAEVKKPGSSVKVSCKASGGTFS
HFR2-
(SEQ ID NO: 8)
WVRQAPGQGLEWMG
HFR3-
(SEQ ID NO: 9)
RVKITADISTRTTYMELSSLRSEDTAVYYCAR
HFR4-
(SEQ ID NO: 10)
WGQGTLVTVSS
LFR1-
(SEQ ID NO: 11)
QSALTQPRSVSGSPGQSVTISC
LFR2-
(SEQ ID NO: 12)
WYQQHPGKAPKLMIY
LFR3-
(SEQ ID NO: 13)
GVPDRFSGSKSGNTASLTISGLQAEDEADYYC
LFR4-
(SEQ ID NO: 14)
FGGGTKLTVL

### Amino Acid Sequence of the Variable Heavy (VH) Region of Anti-EGFR Antibody Clone RAA22 (SEQ ID NO: 15)

QVQLVQSGAEVKKPGSSVKVSCKASGGTFSDNDFSWVRQAPGQGLEWMG
AIVAVFRTETYAQKFQDRVKITADISTRTTYMELSSLRSEDTAVYYCAR
RLMSAISGPGAPLLMWGQGTLVTVSS

### Amino Acid Sequence of the Variable Light (VL) Region of Anti-EGFR Antibody Clone RAA22 (SEQ ID NO: 6):

QSALTQPRSVSGSPGQSVTISCTGTSSDVGGYNYVSWYQQHPGKAPKLM
IYDVSKRPSGVPDRFSGSKSGNTASLTISGLQAEDEADYYCSSYTSSDT
LEIFGGGTKLTVL

### CDR Sequences for Anti-c-Met Antibody Clone B09-GL

HCDR1-
(SEQ ID NO: 17)
DYYIH
HCDR2-
(SEQ ID NO: 18)
WMNPNSGNTGYAQKFQG
HCDR3-
(SEQ ID NO: 19)
GQGYTHS
LCDR1-
(SEQ ID NO: 20)
RASEGIYHWLA
LCDR2-
(SEQ ID NO: 21)
KASSLAS
LCDR3-
(SEQ ID NO: 22)
QQYSNYPPT

### FR Sequences for Anti-c-Met Antibody Clone B09-GL

HFR1-
(SEQ ID NO: 23)
QVQLVQSGAEVKKPGASVKVSCKASGYTFT
HFR2-
(SEQ ID NO: 24)
WVRQATGQGLEWMG
HFR3-
(SEQ ID NO: 25)
RVTMTRDTSISTAYMELSSLRSEDTAVYYCAR
HFR4-
(SEQ ID NO: 26)
WGQGTMVTVSS
LFR1-
(SEQ ID NO: 27)
DIQMTQSPSTLSASVGDRVTITC
LFR2-
(SEQ ID NO: 28)
WYQQKPGKAPKLLIY
LFR3-
(SEQ ID NO: 29)
GVPSRFSGSGSGTEFTLTISSLQPDDFATYYC
LFR4-
(SEQ ID NO: 30)
FGGGTKLEIK

### Amino Acid Sequence of the Variable Heavy (VH) Region of Anti-c-Met Antibody Clone B09-GL (SEQ ID NO: 31):

QVQLVQSGAEVKKPGASVKVSCKASGYTFTDYYIHWVRQATGQGLEWMG
WMNPNSGNTGYAQKFQGRVTMTRDTSISTAYMELSSLRSEDTAVYYCAR
GQGYTHSWGQGTMVTVSS

### Amino Acid Sequence of the Variable Light (VL) Region of Anti-c-Met Antibody Clone B09-GL (SEQ ID NO: 32):

DIQMTQSPSTLSASVGDRVTITCRASEGIYHWLAWYQQKPGKAPKLLIY
KASSLASGVPSRFSGSGSGTEFTLTISSLQPDDFATYYCQQYSNYPPTF
GGGTKLEIK

### Amino Acid Sequence of a Human Immunoglobulin G1 Heavy Chain Constant (CH) Region (SEQ ID NO: 33):

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

### Amino Acid Sequence of a Wild-Type Human Immunoglobulin Kappa Constant Region (SEQ ID NO: 34):

RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

### Amino Acid Sequence of the Heavy Chain of Anti-EGFR in “EGFR-cMET TM” Antibody (SEQ ID NO: 35):

Following substitutions are bolded and underlined:

Triple mutation (TM; L234F, L235E and P331S); “Knob” mutation (T366W); interchain cysteine mutations (F126C and C219V); stabilizing cysteine mutation (S354C), where numbering of residues is according to EU index.

QVQLVQSGAEVKKPGSSVKVSCKASGGTFSDNDFSWVRQAPGQGLEWMGA
IVAVFRTETYAQKFQDRVKITADISTRTTYMELSSLRSEDTAVYYCARRL
MSAISGPGAPLLMWGQGTLVTVSSASTKGPSVCPLAPSSKSTSGGTAALG
CLVKDYFPEPVTVSWNSGALTSGVHTFPAVLQSSGLYSLSSVVTVPSSSL
GTQTYICNVNHKPSNTKVDKRVEPKSVDKTHTCPPCPAPEFEGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPASIEKTISKAKGQP
REPQVYTLPPCREEMTKNQVSLWCLVKGFYPSDIAVEWESNGQPENNYKT
TPPVLDSDGSFFLYSKLTVDKSRWQQGNVFSCSVMHEALHNHYTQKSLSL
SPGK

### Amino Acid Sequence of the Heavy Chain of Anti-c-Met in “EGFR-cMET TM” Antibody (SEQ ID NO: 36):

Following substitutions are bolded and underlined:

Triple mutation (TM; L234F, L235E and P331S); “Hole” mutations (T366S, L368A, and Y407V); and stabilizing cysteine mutation (Y349C), where numbering of residues is according to EU index.

QVQLVQSGAEVKKPGASVKVSCKASGYTFTDYYIHWVRQATGQGLEWMGW
MNPNSGNTGYAQKFQGRVTMTRDTSISTAYMELSSLRSEDTAVYYCARGQ
GYTHSWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPEFEGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPASIEKTISKAKGQPREPQVCTL
PPSREEMTKNQVSLSCAVKGFYPSDIAVEWESNGQPENNYKTTPPVLDSD
GSFFLVSKLTVDKSRWQQGNVFSCSVMHEALHNHYTQKSLSLSPGK

### Amino Acid Sequence of the Light Chain of Anti-EGFR in “EGFR-cMET TM” Antibody (SEQ ID NO: 37):

Following substitutions are bolded and underlined:

S121C and C214V, wherein numbering is according to EU index

QSALTQPRSVSGSPGQSVTISCTGTSSDVGGYNYVSWYQQHPGKAPKLMI
YDVSKRPSGVPDRFSGSKSGNTASLTISGLQAEDEADYYCSSYTSSDTLE
IFGGGTKLTVLGQPKAAPSVTLFPPCSEELQANKATLVCLISDFYPGAVT
VAWKADSSPVKAGVETTTPSKQSNNKYAASSYLSLTPEQWKSHRSYSCQV
THEGSTVEKTVAPTEVS

### Amino Acid Sequence of the Light Chain of Anti-c-Met in “EGFR-cMET TM” Antibody (SEQ ID NO: 38):

DIQMTQSPSTLSASVGDRVTITCRASEGIYHWLAWYQQKPGKAPKLLIYK
ASSLASGVPSRFSGSGSGTEFTLTISSLQPDDFATYYCQQYSNYPPTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Amino Acid Sequence of a Human Immunoglobulin G1 CH Region Modified to Include “Knob” Mutations, a Cysteine to Form a Stabilizing Disulfide Bridge, without a Cysteine Insertion and with the TM (SEQ ID NO: 39):

Following substitutions are bolded and underlined:

Triple mutation (TM; L234F, L235E and P331S); “Knob” mutation (T366W); interchain cysteine mutations (F126C and C219V); stabilizing cysteine mutation (S354C), where numbering of residues is according to EU index.

ASTKGPSVCPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSVDKTHTCPPCPAPEFEGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPASIEKTISKAKGQPREPQVYTLPPCREEMTKNQVSLWC
LVKGFYPSDIAVEWESNGQPENNYKTTPPVLDSDGSFFLYSKLTVDKSRW
QQGNVFSCSVMHEALHNHYTQKSLSLSPGK

Amino Acid Sequence of a Human Immunoglobulin G1 CH Region Modified to Include “Hole” Mutations, a Cysteine to Form a Stabilizing Disulfide Bridge, without a Cysteine Insertion and with the TM (SEQ ID NO: 40):

Following substitutions are bolded and underlined:

Triple mutation (TM; L234F, L235E and P331S); “Hole” mutations (T366S, L368A, and Y407V); and stabilizing cysteine mutation (Y349C), where numbering of residues is according to EU index.

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPEFEGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPASIEKTISKAKGQPREPQVCTLPPSREEMTKNQVSLSC
AVKGFYPSDIAVEWESNGQPENNYKTTPPVLDSDGSFFLVSKLTVDKSRW
QQGNVFSCSVMHEALHNHYTQKSLSLSPGK

### Amino Acid Sequence of a Human Immunoglobulin Lambda Constant Region Modified to Include S121C and C214V Substitutions (SEQ ID NO: 41):

GQPKAAPSVTLFPPCSEELQANKATLVCLISDFYPGAVTVAWKADSSPVK
AGVETTTPSKQSNNKYAASSYLSLTPEQWKSHRSYSCQVTHEGSTVEKTV
APTEVS

### Amino Acid Sequence of the Human EGFR Extracellular Domain (SEQ ID NO: 42):

LEEKKVCQGTSNKLTQLGTFEDHFLSLQRMFNNCEVVLGNLEITYVQRNY
DLSFLKTIQEVAGYVLIALNTVERIPLENLQIIRGNMYYENSYALAVLSN
YDANKTGLKELPMRNLQEILHGAVRFSNNPALCNVESIQWRDIVSSDFLS
NMSMDFQNHLGSCQKCDPSCPNGSCWGAGEENCQKLTKIICAQQCSGRCR
GKSPSDCCHNQCAAGCTGPRESDCLVCRKFRDEATCKDTCPPLMLYNPTT
YQMDVNPEGKYSFGATCVKKCPRNYVVTDHGSCVRACGADSYEMEEDGVR
KCKKCEGPCRKVCNGIGIGEFKDSLSINATNIKHFKNCTSISGDLHILPV
AFRGDSFTHTPPLDPQELDILKTVKEITGFLLIQAWPENRTDLHAFENLE
IIRGRTKQHGQFSLAVVSLNITSLGLRSLKEISDGDVIISGNKNLCYANT
INWKKLFGTSGQKTKIISNRGENSCKATGQVCHALCSPEGCWGPEPRDCV
SCRNVSRGRECVDKCNLLEGEPREFVENSECIQCHPECLPQAMNITCTGR
GPDNCIQCAHYIDGPHCVKTCPAGVMGENNTLVWKYADAGHVCHLCHPNC
TYGCTGPGLEGCPTNGPKIPS

Amino Acid Sequence of the Human c-Met Extracellular Domain (SEQ ID NO: 43):

ECKEALAKSEMNVNMKYQLPNFTAETPIQNVILHEHHIFLGATNYIYVLN
EEDLQKVAEYKTGPVLEHPDCFPCQDCSSKANLSGGVWKDNINMALVVDT
YYDDQLISCGSVNRGTCQRHVFPHNHTADIQSEVHCIFSPQIEEPSQCPD
CVVSALGAKVLSSVKDRFINFFVGNTINSSYFPDHPLHSISVRRLKETKD
GFMFLTDQSYIDVLPEFRDSYPIKYVHAFESNNFIYFLTVQRETLDAQTF
HTRIIRFCSINSGLHSYMEMPLECILTEKRKKRSTKKEVFNILQAAYVSK
PGAQLARQIGASLNDDILFGVFAQSKPDSAEPMDRSAMCAFPIKYVNDFF
NKIVNKNNVRCLQHFYGPNHEHCFNRTLLRNSSGCEARRDEYRTEFTTAL
QRVDLFMGQFSEVLLTSISTFIKGDLTIANLGTSEGRFMQVVVSRSGPST
PHVNFLLDSHPVSPEVIVEHTLNQNGYTLVITGKKITKIPLNGLGCRHFQ
SCSQCLSAPPFVQCGWCHDKCVRSEECLSGTWTQQICLPAIYKVFPNSAP
LEGGTRLTICGWDFGFRRNNKFDLKKTRVLLGNESCTLTLSESTMNTLKC
TVGPAMNKHFNMSIIISNGHGTTQYSTFSYVDPVITSISPKYGPMAGGTL
LTLTGNYLNSGNSRHISIGGKTCTLKSVSNSILECYTPAQTISTEFAVKL
KIDLANRETSIFSYREDPIVYEIHPTKSFISGGSTITGVGKNLNSVSVPR
MVINVHEAGRNFTVACQHRSNSEIICCTTPSLQQLNLQLPLKTKAFFMLD
GILSKYFDLIYVHNPVFKPFEKPVMISMGNENVLEIKGNDIDPEAVKGEV
LKVGNKSCENIHLHSEAVLCTVPNDLLKLNSELNIEWKQAISSTVLGKVI
VQPDQNFT

Amino acid sequence of the cynomolgus monkey c-Met extracellular domain (SEQ ID NO: 44):

ECKEALAKSEMNVNMKYQLPNFTAETAIQNVILHEHHIFLGATNYIYVLN
EEDLQKVAEYKTGPVLEHPDCFPCQDCSSKANLSGGVWKDNINMALVVDT
YYDDQLISCGSVNRGTCQRHVFPHNHTADIQSEVHCIFSPQIEEPNQCPD
CVVSALGAKVLSSVKDRFINFFVGNTINSSYFPHHPLHSISVRRLKETKD
GFMFLTDQSYIDVLPEFRDSYPIKYIHAFESNNFIYFLTVQRETLNAQTF
HTRIIRFCSLNSGLHSYMEMPLECILTEKRKKRSTKKEVFNILQAAYVSK
PGAQLARQIGASLNDDILFGVFAQSKPDSAEPMDRSAMCAFPIKYVNDFF
NKIVNKNNVRCLQHFYGPNHEHCFNRTLLRNSSGCEARRDEYRAEFTTAL
QRVDLFMGQFSEVLLTSISTFVKGDLTIANLGTSEGRFMQVVVSRSGPST
PHVNFLLDSHPVSPEVIVEHPLNQNGYTLVVTGKKITKIPLNGLGCRHFQ
SCSQCLSAPPFVQCGWCHDKCVRSEECPSGTWTQQICLPAIYKVFPTSAP
LEGGTRLTICGWDFGFRRNNKFDLKKTRVLLGNESCTLTLSESTMNTLKC
TVGPAMNKHFNMSIIISNGHGTTQYSTFSYVDPIITSISPKYGPMAGGTL
LTLTGNYLNSGNSRHISIGGKTCTLKSVSNSILECYTPAQTISTEFAVKL
KIDLANRETSIFSYREDPIVYEIHPTKSFISGGSTITGVGKNLHSVSVPR
MVINVHEAGRNFTVACQHRSNSEIICCTTPSLQQLNLQLPLKTKAFFMLD
GILSKYFDLIYVHNPVFKPFEKPVMISMGNENVLEIKGNDIDPEAVKGEV
LKVGNKSCENIHLHSEAVLCTVPNDLLKLNSELNIEWKQAISSTVLGKVI
VQPDQNFT

### Amino Acid Sequence of the Cynomolgus Monkey EGFR Extracellular Domain (SEQ ID NO: 46):

LEEKKVCQGTSNKLTQLGTFEDHFLSLQRMFNNCEVVLGNLEITYVQRNY
DLSFLKTIQEVAGYVLIALNTVERIPLENLQIIRGNMYYENSYALAVLSN
YDANKTGLKELPMRNLQEILHGAVRFSNNPALCNVESIQWRDIVSSEFLS
NMSMDFQNHLGSCQKCDPSCPNGSCWGAGEENCQKLTKIICAQQCSGRCR
GKSPSDCCHNQCAAGCTGPRESDCLVCRKFRDEATCKDTCPPLMLYNPTT
YQMDVNPEGKYSFGATCVKKCPRNYVVTDHGSCVRACGADSYEMEEDGVR
KCKKCEGPCRKVCNGIGIGEFKDTLSINATNIKHFKNCTSISGDLHILPV
AFRGDSFTHTPPLDPQELDILKTVKEITGFLLIQAWPENRTDLHAFENLE
IIRGRTKQHGQFSLAVVSLNITSLGLRSLKEISDGDVIISGNKNLCYANT
INWKKLFGTSSQKTKIISNRGENSCKATGQVCHALCSPEGCWGPEPRDCV
SCQNVSRGRECVDKCNILEGEPREFVENSECIQCHPECLPQVMNITCTGR
GPDNCIQCAHYIDGPHCVKTCPAGVMGENNTLVWKYADAGHVCHLCHPNC
TYGCTGPGLEGCARNGPKIPS

### Amino Acid Sequence of the VH Region of Anti-EGFR Antibody Clone QD6 (SEQ ID NO: 47):

QVQLVQSGAEVKKPGSSVKVSCKASGGTFSDNDFSWVRQAPGQGLEWMGA
IVAVVRTETYAQKFQDRVKITADISTRTTYMELSSLRSEDTAVYYCARRL
MSAISGPGAPLLMWGQGTLVTVSS

Amino acid sequence of the VL region of anti-EGFR antibody clone QD6 (SEQ ID NO: 48):

QSALTQPRSVSGSPGQSVTISCTGTSSDVGGYNYVSWYQQHPGKAPKLMI
YDVSERPSGVPDRFSGSKSGNTASLTISGLQAEDEADYYCFSYTSSDTLE
IFGGGTKLTVL

### Chelating Moiety or Metal Complex Thereof

### Chelating Moieties

Examples of suitable chelating moieties include, but are not limited to, DOTA (1,4,7,10-tetraazacyclododecane-1,4,7,10-tetraacetic acid), DOTMA (1R,4R,7R,10R)-α, α′, α″, α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10-tetra propionic acid), DO3AM-acetic acid (2-(4,7,10-tris(2-amino-2-oxoethyl)-1,4,7,10-tetraazacyclododecan-1-yl)acetic acid), DOTA-GA anhydride (2,2′,2″-(10-(2,6-dioxotetrahydro-2H-pyran-3-yl)-1,4,7,10-tetraazacyclododecane-1,4,7-triyl)triacetic acid, DOTP (1,4,7,10-tetraazacyclododecane-1,4,7,10-tetra(methylene phosphonic acid)), DOTMP (1,4,6,10-tetraazacyclodecane-1,4,7,10-tetramethylene phosphonic acid, DOTA-4AMP (1,4,7,10-tetraazacyclododecane-1,4,7,10-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacyclotetradecane-1,4,8,11-tetrapropionic acid), TETA (1,4,8,11-tetraazacyclotetradecane-1,4,8,11-tetra acetic acid), HEHA (1,4,7,10,13,16-hexaazacyclohexadecane-1,4,7,10,13,16-hexaacetic acid), PEPA (1,4,7,10,13-pentaazacyclopentadecane-N,N′,N″,N″′, N″″-pentaacetic acid), H₄octapa (N,N′-bis(6-carboxy-2-pyridylmethyl)-ethylenediamine-N,N′-diacetic acid), H₂dedpa (1,2-[[6-(carboxy)-pyridin-2-yl]-methylamino]ethane), H₆phospa (N,N′-(methylenephosphonate)-N,N′-[6-(methoxycarbonyl)pyridin-2-yl]-methyl-1,2-diaminoethane), TTHA (triethylenetetramine-N,N,N′,N″,N″′, N″′-hexaacetic acid), DO2P (tetraazacyclododecane dimethanephosphonic acid), HP-DO3A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), octadentate-HOPO (octadentate hydroxypyridinones), or porphyrins.

Preferably, the chelating moiety is selected from DOTA (1,4,7,10-tetraazacyclododecane-1,4,7,10-tetraacetic acid), DOTMA (1R,4R,7R,10R)-α, α′, α″, α″′-tetramethyl-1,4,7,10-tetraazacyclododecane-1,4,7,10-tetraacetic acid, DOTAAM (1,4,7,10-tetrakis(carbamoylmethyl)-1,4,7,10-tetraazacyclododecane), DO3AM-acetic acid (2-(4,7,10-tris(2-amino-2-oxoethyl)-1,4,7,10-tetraazacyclododecan-1-yl)acetic acid), DOTP (1,4,7,10-tetraazacyclododecane-1,4,7,10-tetra(methylene phosphonic acid)), DOTA-4AMP (1,4,7,10-tetraazacyclododecane-1,4,7,10-tetrakis(acetamido-methylenephosphonic acid), NOTA (1,4,7-triazacyclononane-1,4,7-triacetic acid), and HP-DO3A (10-(2-hydroxypropyl)-1,4,7-tetraazacyclododecane-1,4,7-triacetic acid).

In some embodiments, the chelating moiety is DOTA.

In some embodiments, compounds comprise a metal complex of a chelating moiety.

For example, chelating groups may be used in metal chelate combinations with metals, such as manganese, iron, and gadolinium and isotopes (e.g., isotopes in the general energy range of 60 to 10,000 keV), such as any of the radioisotopes and radionuclides discussed herein.

In some embodiments, chelating moieties are useful as detection agents, and compounds comprising such detectable chelating moieties can therefore be used as diagnostic or theranostic agents.

### Radioisotopes and Radionuclides

In some embodiments, the metal complex comprises a radionuclide. Examples of suitable radioisotopes and radionuclides include, but are not limited to, ³H, ¹⁴C, ¹⁵N, ¹⁸F, ³⁵S, ⁴⁴Sc, ⁴⁷Sc, ⁵⁵Co, ⁶⁰Cu, ⁶¹Cu, ⁶²Cu, ⁶⁴Cu, ⁶⁶Ga ⁶⁷Ga, ⁶⁷Cu, ⁶⁸Ga ⁷⁵Br, ⁷⁶Br, ⁷⁷Br, ⁸²Rb, ⁸⁹Zr, ⁸⁶Y, ⁸⁷Y, ⁹⁰Y, ⁹⁷Ru, ⁹⁹Tc, ⁹⁹mTc, ¹⁰⁵Rh, ¹⁰⁹Pd, ¹¹¹In ¹²³I, ¹²⁴I, ¹²⁵I, ¹³¹I, ¹⁴⁹Pm, ¹⁴⁹Tb, ¹⁵³ Sm, ¹⁶⁶Ho ¹⁷⁷Lu, ^{117m}Sn, ¹⁸⁶Re, ¹⁸⁸Re, ¹⁹⁸Au, ¹⁹⁹Au, ²⁰¹Tl, ²⁰³Pb ²¹¹At, ²¹²Pb, ²¹²Bi, ²¹³Bi, ²²³Ra ²²⁵Ac, ^{227Th} and ^{229Th}.

In some embodiments, the metal complex comprises a radionuclide selected from ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga ⁶⁷Ga, ⁶⁸Ga ⁸²Rb, ⁸⁶Y, ⁸⁷Y, ⁸⁹Zr, ⁹⁰Y, ⁹⁷Ru, ⁹⁹Tc, ⁹⁹mTc ¹⁰⁵Rh, ¹⁰⁹Pd, ¹¹¹In, ^{117m}Sn, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au ¹⁹⁹Au, ²⁰¹Tl, ²⁰³Pb, ²¹¹At, ²¹²Pb, ²¹²Bi, ²¹³Bi, ²²³Ra, ²²⁵Ac, ^{227Th} and ^{229Th}.

In certain embodiments, the metal complex comprises a radionuclide selected from ⁶⁸Ga, ⁸⁹Zr, ⁹⁰Y, ¹¹¹In, ¹⁷⁷Lu, and ²²⁵Ac. In certain embodiments, the metal complex comprises a radionuclide of ¹⁷⁷Lu or ²²⁵Ac.

In some embodiments, the radionuclide is an alpha emitter, e.g., Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), or Terbium-149 (¹⁴⁹Tb), or a progeny thereof. In some embodiments, the alpha-emitter is Actinium-225 (²²⁵Ac), or a progeny thereof.

In certain embodiments, the metal complex comprises an alpha emitter of ²²⁵Ac or a progeny thereof.

### Linker

The compounds of this invention comprise the structure of Formula I below:

A-L¹-(L²)ₙ-B Formula I
wherein each of the variables is defined in the SUMMARY section above.

Each of the compounds of Formula I comprises a linker moiety as -L¹-(L²)ₙ-, wherein:
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
each L², independently, has the structure:

—X¹-L³-Z¹ Formula II
wherein:
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—*, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—*, or —NR¹—*; wherein “*” indicates the attachment point to L³, and R¹ is hydrogen, optionally substituted C₁-C₆ alkyl, optionally substituted C₁-C₆ heteroalkyl, or optionally substituted aryl or heteroaryl;
L³ is optionally substituted C₁-C₅₀ alkyl or optionally substituted C₁-C₅₀ heteroalkyl (e.g., (CH₂CH₂O)₂₋₂₀); and
Z¹ is —CH₂-#, —C(O)-#, —C(S)-#, —OC(O)-#, —C(O)O-#, —NR²C(O)-#, —C(O)NR²-#, or —NR²-#, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl.

In some embodiments, L¹ is optionally substituted C₁-C₆ alkyl or optionally substituted C₁-C₆ heteroalkyl. In certain embodiments, L¹ is substituted C₁-C₆ alkyl or substituted C₁-C₆ heteroalkyl, the substituent comprising a heteroaryl group (e.g., six-membered nitrogen-containing heteroaryl). In some embodiments, L¹ is C₁-C₆ alky. For example, L¹ is —CH₂CH₂—. In some embodiments, L¹ is a bond. In some embodiments, L¹ is

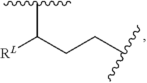

wherein R^{L} is hydrogen or —CO₂H.

In some embodiments, X¹ is —C(O)NR¹—*, —NR¹C(O)—*, or —NR¹—, “*” indicating the attachment point to L³, and R¹ is hydrogen, optionally substituted C₁-C₆ alkyl, optionally substituted C₁-C₆ heteroalkyl, optionally substituted aryl, or optionally substituted heteroaryl. In some embodiments, X¹ is —C(O)NR¹—*, “*” indicating the attachment point to L³, and R¹ is hydrogen.

In some embodiments, L³ is optionally substituted C₁-C₅₀ alkyl (e.g., C₃-C₃₀ alkyl, C₃-C₂₅ alkyl, C₃-C₂₀ alkyl, C₃-C₁₅ alkyl, C₃-C₁₀ alkyl, C₅-C₃₀ alkyl, C₅-C₂₅ alkyl, C₅-C₂₀ alkyl, C₅-C₁₅ alkyl, and C₅-C₁₀ alkyl) or optionally substituted C₁-C₅₀ heteroalkyl (e.g., C₃-C₃₀ heteroalkyl, C₃-C₂₅ heteroalkyl, C₃-C₂₀ heteroalkyl, C₃-C₁₅ heteroalkyl, C₃-C₁₀ heteroalkyl, C5-C₃₀ heteroalkyl, C5-C₂₅ heteroalkyl, C₅-C₂₀ heteroalkyl, C₅-C₁₅ heteroalkyl, and C₅-C₁₀ heteroalkyl). An exemplary C₁-C₅₀ heteroalkyl is C₅-C₃₀ polyethylene glycol (e.g., C₅-C₂₅ polyethylene glycol, C₅-C₂₀ polyethylene glycol, C₅-C₁₅ polyethylene glycol). In certain embodiments, L³ is C₅-C₂₅ polyethylene glycol, C₅-C₂₀ polyethylene glycol, or C₅-C₁₅ polyethylene glycol.

In some embodiments, L³ is optionally substituted C₁-C₅₀ heteroalkyl (e.g., C₁-C₄₀ heteroalkyl, C₁-C₃₀ heteroalkyl, C₁-C₂₀ heteroalkyl, C₂-C₁₈ heteroalkyl, C₃-C₁₆ heteroalkyl, C₄-C₁₄ heteroalkyl, C₅-C₁₂ heteroalkyl, C₆-C₁₀ heteroalkyl, C₈-C₁₀ heteroalkyl, C₄ heteroalkyl, C₆ heteroalkyl, C₈ heteroalkyl, C₁₀ heteroalkyl, C₁₂ heteroalkyl, C₁₆ heteroalkyl, C₂₀ heteroalkyl, or C₂₄ heteroalkyl).

In some embodiments, L³ is optionally substituted C₁-C₅₀ heteroalkyl comprising a polyethylene glycol (PEG) moiety comprising 1-20 oxyethylene (—O—CH₂—CH₂—) units, e.g., 2 oxyethylene units (PEG2), 3 oxyethylene units (PEG3), 4 oxyethylene units (PEG4), 5 oxyethylene units (PEG5), 6 oxyethylene units (PEG6), 7 oxyethylene units (PEG7), 8 oxyethylene units (PEG8), 9 oxyethylene units (PEG9), 10 oxyethylene units (PEG10), 12 oxyethylene units (PEG12), 14 oxyethylene units (PEG14), 16 oxyethylene units (PEG16), or 18 oxyethylene units (PEG18).

In certain embodiments, L³ is optionally substituted C₁₋₅₀ heteroalkyl comprising a polyethylene glycol (PEG) moiety comprising 1-20 oxyethylene (—O—CH₂—CH₂—) units or portions thereof. For example, L³ comprises PEG3 as shown below:

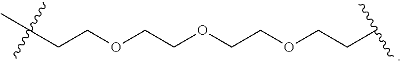 [SMILES: *CCOCCOCCOCC*]

In some embodiments, L³ is (CH₂CH₂O)ₘ(CH₂)_{w}, and m and w are each independently an integer between 0 and 10 (inclusive), and at least one of m and w is not 0.

In some embodiments, L³ is substituted C₁-C₅₀ alkyl or substituted C₁-C₅₀ heteroalkyl, the substituent comprising a heteroaryl group (e.g., six-membered nitrogen-containing heteroaryl).

In some embodiments, Z¹ is CH₂, C═O, or NR¹; wherein R¹ is H, optionally substituted C₁-C₆ alkyl, optionally substituted C₁-C₆ heteroalkyl, optionally substituted aryl, or optionally substituted heteroaryl.

In certain embodiments, A-L¹-(L²)ₙ-B can be represented by Formula I-a, or a metal complex thereof:

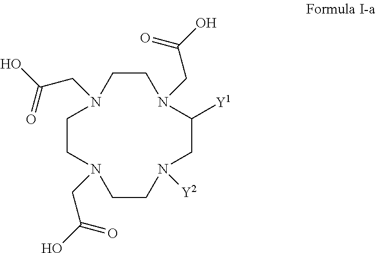

wherein Y¹ is —CH₂OCH₂(L²)ₙ-B, —C(O)(L²)ₙ-B, or —C(S)(L²)ₙ-B and Y² is —CH₂CO₂H; or wherein Y¹ is H and Y² is L¹-(L²)ₙ-B.

### Cross-Linking Groups

In some embodiments, compounds (e.g., radioimmunoconjugates) are synthesized using bifunctional chelates that comprise a chelate, a linker, and a cross-linking group. Once the compound (e.g., radioimmunoconjugate) is formed, the cross-linking group may be absent from the compound (e.g., radioimmunoconjugate).

In some embodiments, compounds (e.g., radioimmunoconjugates) comprise a cross-linking group instead of, or in addition to, the targeting moiety (e.g., in some embodiments, B in Formula I comprises a cross-linking group).

A cross-linking group is a reactive group that is able to join two or more molecules by a covalent bond. Cross-linking groups may be used to attach the linker and chelating moiety to a therapeutic or targeting moiety. Cross-linking groups may also be used to attach the linker and chelating moiety to a target in vivo. In some embodiments, the cross-linking group is an amino-reactive, methionine reactive or thiol-reactive cross-linking group, or a comprises sortase recognition sequence. In some embodiments, the amino-reactive or thiol-reactive cross-linking group comprises an activated ester such as a hydroxysuccinimide ester, 2,3,5,6-tetrafluorophenol ester, 4-nitrophenol ester or an imidate, anhydride, thiol, disulfide, maleimide, azide, alkyne, strained alkyne, strained alkene, halogen, sulfonate, haloacetyl, amine, hydrazide, diazirine, phosphine, tetrazine, isothiocyanate, or oxaziridine. In some embodiments, the sortase recognition sequence may comprise of a terminal glycine-glycine-glycine (GGG) and/or LPTXG amino acid sequence, where X is any amino acid. A person having ordinary skill in the art will understand that the use of cross-linking groups is not limited to the specific constructs disclosed herein, but rather may include other known cross-linking groups.

### Pharmaceutical Compositions

In one aspect, the present disclosure provides pharmaceutical compositions comprising compounds disclosed herein. Such pharmaceutical compositions can be formulated for use in a variety of drug delivery systems. One or more physiologically acceptable excipients or carriers can also be included in a pharmaceutical composition for proper formulation. Non-limiting examples of suitable formulations compatible for use with the present disclosure include those described in Remington's Pharmaceutical Sciences, Mack Publishing Company, Philadelphia, PA, 17th ed., 1985. For a brief review of methods for drug delivery, See, e.g., Langer (Science. 249:1527-1533, 1990).

Pharmaceutical compositions may be formulated for any of a variety of routes of administration discussed herein (See, e.g., the “Administration and Dosage” subsection herein), Sustained release administration is contemplated, by such means as depot injections or erodible implants or components. Thus, the present disclosure provides pharmaceutical compositions that include agents disclosed herein (e.g., radioimmunoconjugates) dissolved or suspended in an acceptable carrier, preferably an aqueous carrier, e.g., water, buffered water, saline, or PBS, among others. In some embodiments, pharmaceutical compositions contain pharmaceutically acceptable auxiliary substances to approximate physiological conditions, such as pH adjusting and buffering agents, tonicity adjusting agents, wetting agents, or detergents, among others. In some embodiments, pharmaceutical compositions are formulated for oral delivery and may optionally contain inert ingredients such as binders or fillers for the formulation of a unit dosage form, such as a tablet or a capsule. In some embodiments, pharmaceutical compositions are formulated for local administration and may optionally contain inert ingredients such as solvents or emulsifiers for the formulation of a cream, an ointment, a gel, a paste, or an eye drop.

In some embodiments, provided pharmaceutical compositions are sterilized by conventional sterilization techniques, e.g., may be sterile filtered. Resulting aqueous solutions may be packaged for use as is, or lyophilized. Lyophilized preparations can be, for example, combined with a sterile aqueous carrier prior to administration. The pH of preparations typically will be between 3 and 11, more preferably between 5 and 9 or between 6 and 8, and most preferably between 6 and 7, such as 6 to 6.5. Resulting compositions in solid form may be packaged, for example, in multiple single dose units, each containing a fixed amount of the above-mentioned agent or agents, such as in a sealed package of tablets or capsules. Pharmaceutical compositions in solid form can also be packaged in a container for a flexible quantity, such as in a squeezable tube designed for a topically applicable cream or ointment.

### Methods of Treatment

In one aspect, the present disclosure provides methods of treatment comprising administering to a subject in need thereof a compound (e.g., radioimmunoconjugate) as disclosed herein.

### Subjects

In some disclosed methods, a therapy (e.g., comprising a therapeutic agent) is administered to a subject. In some embodiments, the subject is a mammal, e.g., a human.

In some embodiments, the subject has cancer or is at risk of developing cancer. For example, the subject may have been diagnosed with cancer. For example, the cancer may be a primary cancer or a metastatic cancer. Subjects may have any stage of cancer, e.g., stage I, stage II, stage III, or stage IV with or without lymph node involvement and with or without metastases. Provided compounds (e.g., radioimmunoconjugates) and compositions may prevent or reduce further growth of the cancer and/or otherwise ameliorate the cancer (e.g., prevent or reduce metastases). In some embodiments, the subject does not have cancer but has been determined to be at risk of developing cancer, e.g., because of the presence of one or more risk factors such as environmental exposure, presence of one or more genetic mutations or variants, family history, etc. In some embodiments, the subject has not been diagnosed with cancer.

In some embodiments, the cancer is any cancer that comprises cells expressing EGFR and cMET. In certain embodiments, the cancer is lung cancer, colorectal cancer, pancreatic cancer, or head and neck cancer.

### Administration and Dosage

Compounds (e.g., radioimmunoconjugates) and pharmaceutical compositions thereof disclosed herein may be administered by any of a variety of routes of administration, including systemic and local routes of administration

Systemic routes of administration include parenteral routes and enteral routes. In some embodiments, compounds (e.g., radioimmunoconjugates) or pharmaceutical compositions thereof are administered by a parenteral route, for example, intravenously, intraarterially, intraperitoneally, subcutaneously, intracranially, or intradermally. In some embodiments, compounds (e.g., radioimmunoconjugates) or pharmaceutical compositions thereof are administered intravenously. In some embodiments, compounds (e.g., radioimmunoconjugates) or pharmaceutical compositions thereof are administered by an enteral route of administration, for example, trans-gastrointestinal, or orally.

Local routes of administration include, but are not limited to, peritumoral injections and intratumoral injections.

Pharmaceutical compositions can be administered for radiation treatment planning, diagnostic, and/or therapeutic treatments. When administered for radiation treatment planning or diagnostic purposes, the compound (e.g., radioimmunoconjugate) may be administered to a subject in a diagnostically effective dose and/or an amount effective to determine the therapeutically effective dose. In therapeutic applications, pharmaceutical compositions may be administered to a subject (e.g., a human) already suffering from a condition (e.g., cancer) in an amount sufficient to cure or at least partially arrest the symptoms of the disorder and its complications. An amount adequate to accomplish this purpose is defined as a “therapeutically effective amount,” an amount of a compound sufficient to substantially improve at least one symptom associated with the disease or a medical condition. For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but may, for example, provide a treatment for a disease or condition such that the onset of the disease or condition is delayed, hindered, or prevented, such that the disease or condition symptoms are ameliorated, or such that the term of the disease or condition is changed. For example, the disease or condition may become less severe and/or recovery is accelerated in an individual. In some embodiments, a subject is administered a first dose of a compound (e.g., radioimmunoconjugate) or composition in an amount effective for radiation treatment planning, then administered a second dose or set of doses of the compound (e.g., radioimmunoconjugate) or composition in a therapeutically effective amount.

For treating cancer comprising cells expressing EGFR and cMET, the method of this invention typically comprises administering to a subject (e.g., a human) in need thereof a first dose of a compound or composition provided above in an amount effective for radiation treatment planning, followed by administering subsequent doses of a compound or composition provided above in a therapeutically effective amount.

In some embodiments, the compound or composition administered in the first dose and the compound or composition administered in the second dose are the same.

In some embodiments, the compound or composition administered in the first dose and the compound or composition administered in the second dose are different.

Therapeutically effective amounts may depend on the severity of the disease or condition and other characteristics of the subject (e.g., weight). Therapeutically effective amounts of disclosed compounds (e.g., radioimmunoconjugates) and compositions for subjects (e.g., mammals such as humans) can be determined by the ordinarily-skilled artisan with consideration of individual differences (e.g., differences in age, weight and the condition of the subject).

In some embodiments, disclosed compounds (e.g., radioimmunoconjugates) exhibit an enhanced ability to target cancer cells. In some embodiments, the effective amount of disclosed compounds (e.g., radioimmunoconjugates) is lower than (e.g., less than or equal to about 90%, 75%, 50%, 40%, 30%, 20%, 15%, 12%, 10%, 8%, 7%, 6%, 5%, 4%, 3%, 2%, 1%, 0.5%, or 0.1% of) the equivalent dose for a therapeutic effect of the unconjugated, and/or non-radiolabeled targeting moiety.

Single or multiple administrations of pharmaceutical compositions disclosed herein including an effective amount can be carried out with dose levels and pattern being selected by the treating physician. Dose and administration schedule can be determined and adjusted based on the severity of the disease or condition in the subject, which may be monitored throughout the course of treatment according to the methods commonly practiced by clinicians or those described herein.

The following specific examples are to be construed as merely illustrative, and not limitative of the remainder of the disclosure in any way whatsoever.

### EXAMPLES

### Example 1—RAA22/B09 DuetMab Design and Construction

This example describes the creation of bispecific antibody molecules that are capable of binding both EGFR and c-Met.

### 1.1 Isolation and Identification of Anti-cMET Antibody 0021U3-B09

cMET-specific scFv antibodies were isolated from a large naïve human scFv phage display library in a series of repeated panning selection cycles on recombinant mammalian expressed biotinylated monomeric human cMET (MedImmune) essentially as described (Vaughan, 1996). ScFvs from the round 2 of the selection output were expressed in the bacterial periplasm and screened for their ability to inhibit the binding of the human cMET receptor with the HGF ligand in a HGF:cMET HTRF® (Homogeneous Time-Resolved Fluorescence) ligand receptor inhibitory binding assay. Top hits exhibiting strong inhibitory effect were selected and subjected to DNA sequencing. Unique genes were then converted to human immunoglobulin G2 (IgG2) antibodies and produced in mammalian cells essentially as described (Persic; 1997). The purified antibodies were then ranked based on their inhibitory effect in the HGF:cMET HTRF® binding assay. The most potent antibody, 0021U3-B09, was selected for further characterization.

### 1.2 Optimization of Anti-cMET Antibody 0021U3-B09.

To minimize potential immunogenicity, non-Vernier framework residues (Foote and Winter 1992) in the variable framework regions of 0021U3-B09 were targeted specifically and altered to match the closest human germline sequence. In the VH region, seven amino acid residues were mutated to match the reference human germline sequence IGHV1-8*01. In the VL region, three residues were mutated to match the reference human germline sequence IGKV1-5*03. All residues in VH and VL regions were successfully changed to the germline residues without loss of activity. 0021U3-B09 was affinity optimized using a hybridization-based mutagenesis method essentially as described (Kunkel 1985). A large scFv library derived from 0021U3-B09 sequence was created by oligonucleotide-directed mutagenesis of the VH complementarity determining regions 3 (CDR3) using standard molecular biology techniques. The library was subjected to affinity-based solution phase selections to select variants with a higher affinity to human and cynomolgus cMET antigens. Crude scFv-containing periplasmic extracts from the CDR-targeted selection outputs were screened for improved inhibitory activity in the HGF:cMET HTRF® binding assay. Variants exhibiting significantly improved inhibitory effect compared to parent 0021U3-B09, were subjected to DNA sequencing and unique genes were converted to human IgG2. The purified antibodies were then ranked based on their inhibitory effect. The most potent antibody, B09-57, was selected for further characterization.

### 1.3 Isolation and Identification of Anti-EGFR Antibody Tdev-0004.

EGFR-specific scFv antibodies were isolated from a large naïve human scFv phage display library in a series of repeated panning selection cycles on recombinant mammalian expressed biotinylated monomeric human EGFR (MedImmune) essentially as described (Vaughan, 1996). ScFv-displaying phage from the round 3 of the selection output were screened for their binding to human and cynomolgus EGFR in ELISA. Top hits showing cross reactivity were selected and subjected to DNA sequencing. Unique genes were then converted to human immunoglobulin G1 (IgG1) antibodies and produced in mammalian cells essentially as described (Persic, 1997). The purified antibodies were then ranked based on their binding to the EGFR-expressing cell line, A431, by flow cytometry. Antibody Tdev-0004 exhibiting specific cell binding was selected for further characterization.

### 1.4 Optimization of Anti-EGFR Antibody Tdev-0004.

Variant RAA22 and QD6, were derived by optimizing the anti-EGFR Tdev-0004 mAb. The VL frameworks of Tdev-0004 had 100% match to the reference human germline sequence IGLV2-11*01/IGLJ2 (see https://www.ncbi.nlm.nih.gov/projects/igblast/Idlink.cgi?seqname=IGLV2-11*01&taxid=9606&dbname=IG_DB %2Fimgt.Homo_sapiens.V.f.orf.p), however, the VH frameworks had 79% homology with the closest human germline IGHV1-69*01/JH4 (see https://www.ncbi.nlm.nih.gov/projects/igblast/Idlink.cgi?segname=IGHV1-69*01&taxid=9606&dbname=IG_DB %2Fimgt.Homo_sapiens.V.f.orfp). To minimize potential immunogenicity, the VH region was initially fully germlined by mutating all 13 non-germline framework residues. Upon germlining, the binding of the fully germlined variant to cynomolgus EGFR was significantly impaired. To restore the binding to cynomolgus EGFR, four non-germline residues; K68, I73, R76 and T78 were selectively back mutated. Amino acid residues are numbered by Kabat numbering system (Kabat and Wu 1991). The resulting, partially germlined variant, named H4, was used as a template sequence for the affinity optimization. Variant H4 was affinity optimized by parsimonious mutagenesis of all six CDRs using a QuikChange Lightning Multi Site-Directed Mutagenesis Kit (Agilent), according to the manufacturer's instructions. Single amino acid mutagenized VH and VL libraries were expressed in bacteria as Fab fragments and screened for improved binding to human and cynomolgus EGFR in ELISA. Variants exhibiting improved binding compared to parent H4 were subjected to DNA sequencing and unique genes were converted to human IgG1. Variant RAA22 was identified with a single mutation in CDRH3. To further improve the affinity, individual positive mutations were combined to create a combinatorial library that was screened for variants with enhanced binding to human and cynomolgus EGFR. Variant QD6 was identified with four combined mutations in CDRL2, CDRL3 and CDRH3.

1.5 Generation of Monovalent Bispecific Anti-EGFR cMET DuetMab Antibodies.

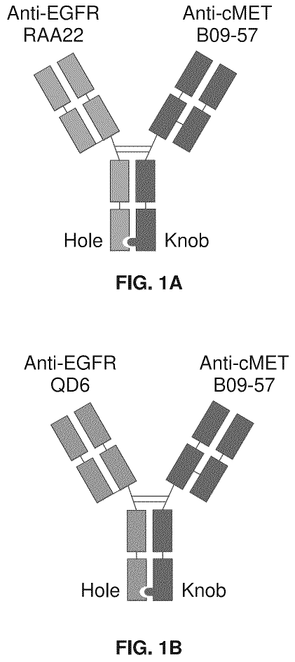

The variable domains of the anti-cMET mAb B09-57 and anti-EGFR mAbs RAA22 and QD6 were utilized for the construction of monovalent bispecific anti-EGFR/cMET antibodies on the backbone of the DuetMab platform (Mazor, 2015). Specifically, the VH gene of the anti-cMET B09-57 was inserted into a human gamma-1 constant heavy chain carrying the “Knob” mutation (T366W) and the alternative interchain cysteine mutations (F126C and C219V). The VL gene of B09-57 was inserted in frame into a human Kappa constant domain carrying the corresponding alternative interchain cysteine mutations (S121C and C214V) designed to pair with the “Knob” heavy chain. Similarly, the VH genes of the anti-EGFR RAA22 and affinity optimized QD6 were inserted into a human gamma-1 constant heavy chain carrying the “Hole” mutations (T366S, L368A, and Y407V), while the VL genes of RAA22 and B09-57 were inserted in frame into a human Lambda constant domain designed to pair with the “Hole” heavy chain. In addition, two residues in the CH₃ domains of “Knob” and “Hole” heavy chains were mutated to cysteine (S354C in “Knob” and Y349C in “Hole”) to form a stabilizing disulfide bridge. The Fc domain was further engineered to carry a Cysteine insertion after Serine 239 (C239i/“Maia”) designed to enable site-specific conjugation of maleimide-bearing cytotoxic drugs (Dimasi, 2017). Amino acid residues are numbered by Kabat numbering system (Kabat and Wu 1991). The assembled monovalent bispecific anti-EGFR/cMET DuetMab antibodies were designated as RAA22/B09-57 and QD6/B09-57 (FIG. 1). DuetMab antibodies were produced from mammalian cells as previously described (Mazor, 2017).

It was also recognised that it may be possible to mitigate immune toxicities and improve pharmacokinetics if the effector functions of the Fc backbone were reduced or removed. We therefore also introduced the “triple mutant (TM)” of L234F/L235E/P331S (EU numbering) that has previously been shown to reduce Fc effector functions in antibody molecules (Organesyan, 2008; Hay, 2016). These TM constructs were engineered such that the c-MET arm carried the “Hole” mutation and the EGFR arm carried the “Knob” mutation.

The generated “EGFR-cMET TM” DuetMab, comprising variable regions from RAA22 and B09 with the TM introduced has the amino acid sequences set forth in the following table:

| | “EGFR-cMET TM” |
|---|---|
| EGFR heavy chain | 35 |
| c-Met heavy chain | 36 |
| EGFR light chain | 37 |
| c-Met light chain | 38 |

### Example 2-Biochemical and Biophysical Properties

This example tests various biochemical and biophysical properties of the RAA22, QD6 and B09-57 monoclonal antibodies and RAA22/B09-57 and QD6/B09-57 bispecific antibodies molecules, including their binding affinity to EGFR and c-Met, respectively and their ability to bind both antigens simultaneously.

2.1 Binding Affinity of DuetMabs and Parental mAbs for EGFR and MET.

The kinetic rate constants (kₒₙ and k_{off}), and equilibrium dissociation constants (Kd) of EGFR-cMET DuetMAbs for recombinant human, cynomolgus monkey, and murine EGFR and cMET antigens were determined at 25° C. by SPR using an antibody capture assay on a BIAcore T200 instrument (GE Healthcare, Pittsburgh, PA). Mouse anti-human IgG was immobilized on a CM4 sensor chip with a final surface density of ˜2000 resonance units (RUs). A reference flow cell surface was also prepared on this sensor chip using identical immobilization protocol. Test and control article antibodies were prepared at 5-20 nM in instrument buffer (HBS-EP buffer; 0.01M HEPES, pH 7.4, 0.15M NaCl, 3 mM EDTA, and 0.005% P-20), along with 3-fold serial dilutions of purified EGFR (0.27-200 nM human, 0.4-900 nM cyno, and 4-1000 nM murine) or cMET proteins (0.27-66 nM human and 0.27-22 nM cyno) in instrument buffer. A sequential approach was utilized for kinetic measurements. Antibodies were first injected over the capture surface, at a flow rate of 10 μL/minute. Once the binding of the captured antibody stabilized, a single concentration of the analyte was injected over both capture and reference surfaces, at a flow rate of 75 μL/minute. The resulting binding response curves yielded the association phase data. Following the injection of analyte, the flow was then switched back to instrument buffer for 15 minutes to permit the collection of dissociation phase data, followed by a 1-minute pulse of 10 mM glycine, pH 1.5, to regenerate the antibody-captured surface on the chip. Binding responses against test and control article antibodies were recorded from duplicate injections of each concentration of analyte. In addition, several buffer injections were interspersed throughout the injection series. Select buffer injections were used along with the reference cell responses to correct the raw data sets for injection artifacts and/or nonspecific binding interactions, commonly referred to as “double referencing”. Corrected binding data were globally fit to a 1:1 binding model (Biacore T200 Evaluation software 2.0, GE Healthcare, Pittsburgh, PA). The calculated kinetic parameters (kₒₙ and k_{off}) and Kd determined as k_{off}/kₒₙ are shown in Table 1.

TABLE 1
Kinetics of DuetMabs and parental
IgGs to EGFR and cMET Antigens

| Antibody | Antigen | Kₒₙ (M⁻¹ s⁻¹) | K_{off} (s⁻¹) | Kd (nM) |
|---|---|---|---|---|
| RAA22 | Human EGFR | 4.41 × 10⁴ | 2.06 × 10⁻³ | 46.6 |
| IgG | Cynomolgus | 3.54 × 10⁴ | 6.01 × 10⁻³ | 169.8 |
| | monkey EGFR | | | |
| | Mouse EGFR | 4.03 × 10⁴ | 1.90 × 10⁻² | 488.0 |
| | Human cMET | | | |
| | Cynomolgus | No binding detected at 200 nM | | |
| | monkey cMET | | | |
| | Mouse cMET | | | |
| QD6 IgG | Human EGFR | 1.48 × 10⁵ | 2.92 × 10⁻⁴ | 2.0 |
| | Cynomolgus | 1.14 × 10⁵ | 2.88 × 10⁻⁴ | 2.5 |
| | monkey EGFR | | | |
| | Mouse EGFR | 1.67 × 10⁵ | 7.35 × 10⁻⁴ | 4.4 |
| | Human cMET | | | |
| | Cynomolgus | ND | | |
| | monkey cMET | | | |
| | Mouse cMET | | | |
| B09-57 | Human EGFR | | | |
| IgG | Cynomolgus | No binding detected at 200 nM | | |
| | monkey EGFR | | | |
| | Mouse EGFR | | | |
| | Human cMET | 5.63 × 10⁵ | 8.82 × 10⁻⁴ | 1.6 |
| | Cynomolgus | 1.04 x 10⁶ | 1.94 × 10⁻³ | 1.9 |
| | monkey cMET | | | |
| | Mouse cMET | No binding detected at 200 nM | | |
| RAA22/ | Human EGFR | 4.47 × 10⁴ | 2.01 × 10⁻³ | 45.0 |
| B09-57 | Cynomolgus | 2.78 × 10⁴ | 5.48 × 10⁻³ | 197.0 |
| DuetMab | monkey EGFR | | | |
| | Mouse EGFR | 3.54 × 10⁴ | 2.06 × 10⁻² | 575.4 |
| | Human cMET | 4.43 × 10⁵ | 9.86 × 10⁻⁴ | 2.2 |
| | Cynomolgus | 9.74 × 10⁵ | 2.12 × 10⁻³ | 2.2 |
| | monkey cMET | | | |
| | Mouse cMET | ND | | |
| QD6/ | Human EGFR | 6.35 × 10⁴ | 3.74 × 10⁻⁴ | 5.9 |
| B09-57 | Cynomolgus | 2.10 × 10⁵ | 5.85 × 10⁻⁴ | 2.8 |
| DuetMab | monkey EGFR | | | |
| | Mouse EGFR | 1.26 × 10⁵ | 7.80 × 10⁻⁴ | 6.2 |
| | Human cMET | 4.25 × 10⁵ | 6.96 × 10⁻⁴ | 1.6 |
| | Cynomolgus | 9.58 × 10⁵ | 2.06 × 10⁻³ | 2.2 |
| | monkey cMET | | | |

| | |
|---|---|
| Mouse cMET | ND |

^{a}ND: not determined. Kinetic measurements to soluble monomeric forms of EGFR and cMET were performed using a BIACore instrument. Kd were calculated as the ratio of k_{off}/kₒₙ from a non-linear fit of the data.

As can be seen from the above data, bispecific antibody molecule QD6/B09-57 binds human cMet with a high affinity (˜2 nM Kd) and human EGFR with a high affinity (˜6 nM Kd), whilst bispecific antibody molecule RAA22/B09-57 binds human c-Met with a similarly high affinity (˜2 nM Kd) but binds human EGFR with a reduced affinity (˜45 nM Kd) in comparison to QD6/B09-57.

2.2 Concurrent Binding of DuetMabs to EGFR and cMET.

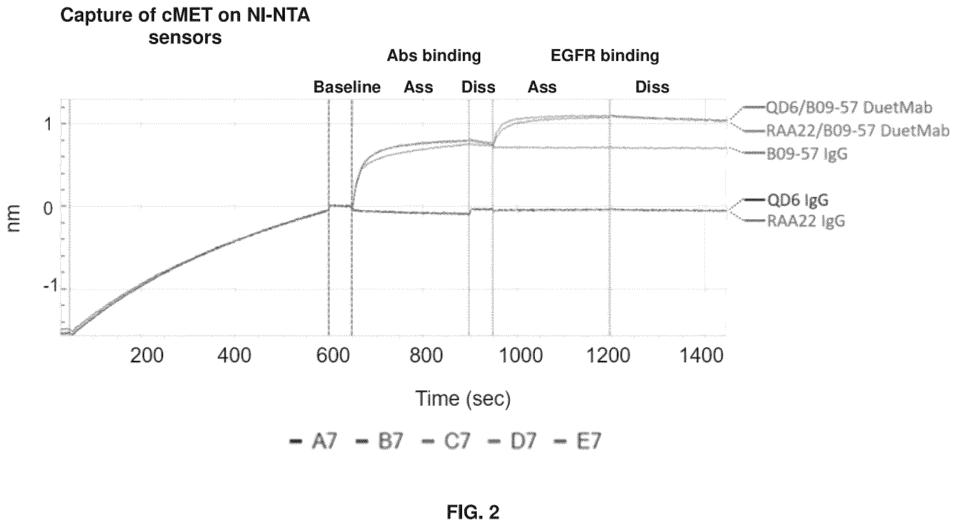

Concurrent binding studies to recombinant human EGFR and cMET proteins were measured by biolayer interferometry on an Octet384 instrument essentially as described (Mazor, 2015). Briefly, His-tagged cMET antigen at 5 μg/mL in assay buffer [PBS pH 7.2, 3 mg/mL bovine serum albumin (BSA), 0.05% (v/v) Tween 20] was initially captured on NI-NTA biosensors. Following a washing step to remove any unbound protein, the respective loaded biosensors were subjected to successive association and dissociation interactions, first with 66 nM of the antibodies and then with the EGFR antigen at 500 nM. Association and dissociation curves were calculated from a non-linear fit of the data using the Octet384 software v.9.0. As shown in FIG. 2 the DuetMabs demonstrated simultaneous binding to both antigens while the parental anti-cMET IgG exhibited specific binding only to cMET and the two anti-EGFR IgGs exhibited no binding to the cMET loaded sensors.

2.3 EGFR and cMet Specificity

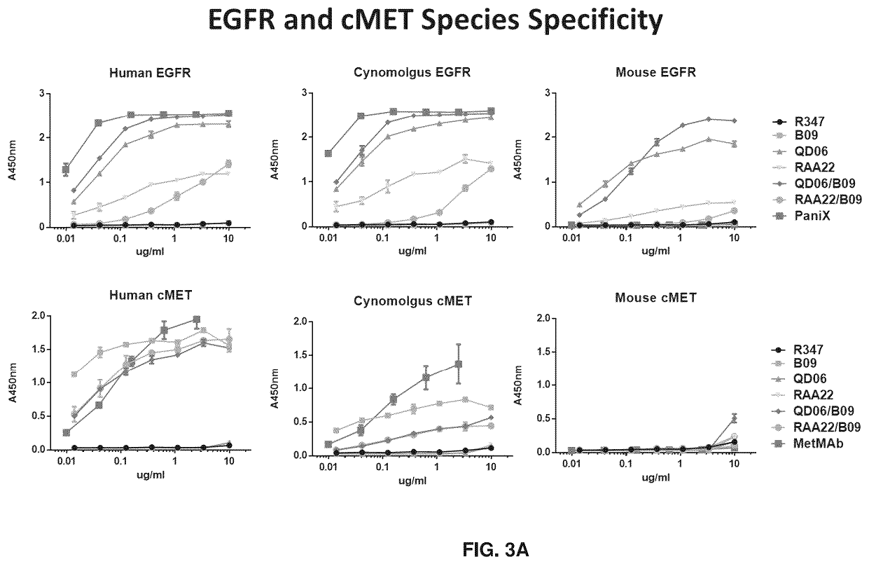

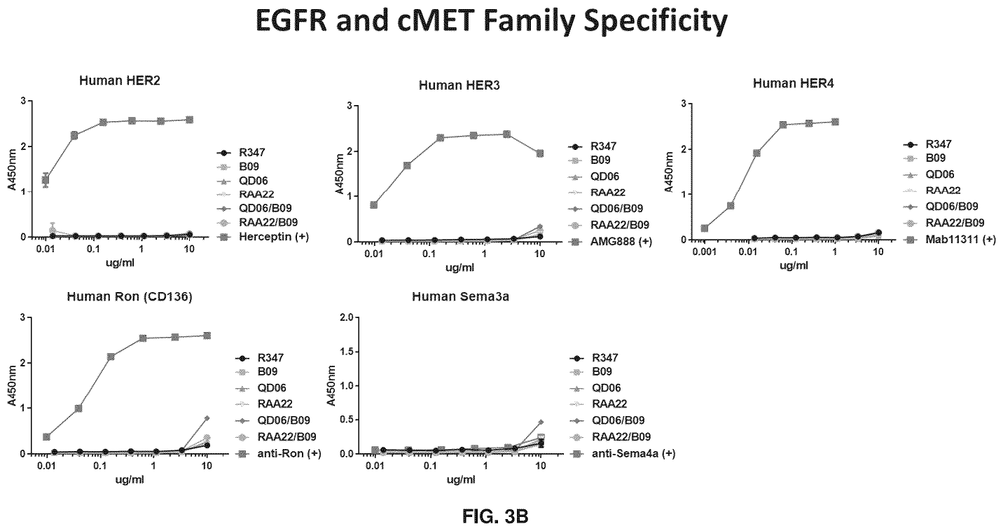

Specificity for EGFR and cMET species paralogs and closely related family members was determined by ELISA. Briefly, antigen solutions were prepared in PBS at 1 μg/mL and 50 microliters was coated onto half area ELISA assay plates. Plates were washed and blocked with 1% BSA in PBS containing 0.005% Tween-20 (PBS-T) for one hour at room temperature. The wells were washed 4 times in PBS-T. As set out in FIG. 3, the primary antibodies used where: R374 (a non-binding IgG1 isotype control antibody), B09 (anti-cMET antibody), QD6 (anti-EGFR antibody), RAA22 (anti-EGFR antibody), QD6/B09 (bispecific EGFR/c-MET DuetMAb), RAA22/B09 (bispecific EGFR/c-MET DuetMAb), PaniX (anti-EGFR antibody), MetMab (anti-cMET antibody) and Mab 11311 (anti-HER4 antibody). Wells were incubated with 50 microliters of the indicated primary antibodies diluted in PBS-T in a 1:3 dilution series, starting at 10 μg/mL and ending at 0.002 μg/mL, except for the HER4 binding mAb control, MAB1131, for which the series started at 1 μg/mL. The wells were washed 4 times in PBS-T, then 50 μl of goat anti-human Fab HRP-labeled secondary antibody, diluted 1:5000 in PBS-T, was added to each well and incubated for one hour at room temperature. 50 microliters of TMB substrate solution was added to all wells and incubated at room temperature for 5-30 min, until intense signal was observed in the positive control wells. 50 microliters of TMB stop solution was added to all wells and the absorbance was read at 450 nm on a SpectraMax M5 microplate reader. Data were analyzed in the SoftMax Pro 5 software and plotted using GraphPad Prism 7 graphing software.

To determine the species cross reactivity, ELISA assays were carried out as described above. As shown in FIG. 3A, the high affinity EGFR IgG, QD6, as well as the monovalent bispecific EGFR/cMET DuetMAb, QD6/B09, bound to human, cynomolgus monkey, and mouse EGFR and gave robust signals in the ELISA assay. In contrast, the lowered affinity EGFR IgG, RAA22, bound more weakly to human, cynomolgus monkey, and mouse EGFR compared to QD6. Binding to mouse EGFR was weak, but detectable. The corresponding monovalent bispecific EGFR/cMET DuetMAb, RAA22/B09, showed still weaker binding to human and cynomolgus monkey EGFR relative to the bivalent parental IgG, RAA22, and nominal binding to mouse EGFR. The cMET IgG, B09, as well as all of the bispecific variants, showed comparable binding to human and cynomolgus monkey cMET. There was no detectable binding of any of the antibodies to mouse cMET. These results are consistent with the binding kinetics determined by surface plasmon resonance measurements on the BIAcore instrument (Table 1 above).

As shown in FIG. 3B none of the parental IgG or derivative bispecific antibodies showed appreciable binding to any of the EGFR HER family proteins, HER2, HER3, or HER4. Similarly, none of the antibodies showed significant binding to the cMET family members, Ron (CD136) or Semaphorin 3a.

These results demonstrate that the parental IgG's and the resulting bispecific antibodies bind specifically to their cognate targets, with no detectable binding to closely related family species.

### Example 3—Monitoring Antibody Internalization and Trafficking to Acidified Compartments Using a pH Sensitive Dye

Target expression in normal, non-tumor tissues can lead to toxicities that reduce the therapeutic window of the antibody conjugates by decreasing payload delivery to the target tissue. The design of the bispecific antibodies or antigen-binding fragments thereof of compounds (e.g., radioimmunoconjugates) of the present disclosure are intended to minimize the impact of the conjugate in normal tissues that exhibit little or no co-expression of the targets while maximizing the delivery of the antibody molecule (i.e., the payload delivery vehicle) to tumor cells that co-express the two targets. To assess whether dual targeting using the EGFR affinity reduced bispecific antibody, RAA22/B09, provided a selectivity advantage relative to single target engagement, we carried out studies using pH sensitive dye labeled antibodies to compare internalization efficiency of the bispecific mAb versus the monovalent parental antibodies comprising the bispecific.

Visualization of antibody internalization and trafficking to acidified intracellular compartments, such as lysosomes and endosomes, was facilitated using antibodies labeled with pHAb pH sensitive dye (Promega). This dye exhibits very low fluorescence at pH>7, but becomes strongly fluorescent at acidic pH, reaching a maximum at approximately pH 5. Briefly, antibodies were labeled with pHAb amine reactive dye, according to the manufacturer's recommendations. The antibodies that were labeled were: R347 IgG1 isotype control, monovalent bispecific control antibodies anti-EGFR antibody RAA22/R347 and anti-cMET B09/R347, as well as EGFR/cMET monovalent bispecific antibody RAA22/B09. NCI-H1975 lung cancer cells, which co-express modest levels of EGFR (˜33,000 relative receptor density) and cMET (˜50,000 relative receptor density), were plated into clear bottomed, black walled 96-well assay plates in 100 microliter volumes at a density of 2×10⁵ cells/mL in RPMI growth medium supplemented with 10% fetal bovine serum. The plates were cultured in a humidified incubator overnight at 37° C. and 5% CO₂. The plates were then chilled on ice for 30 min prior to addition of pHAb labeled bispecific and monovalent control antibodies at various concentrations in pre-chilled growth medium. The cultures were chilled on ice for another 30 minutes, and then the fluorescence was read on an Operetta High Content Imaging system using the Cy3 filter and this initial reading was designated as time zero. The plates were moved back to the 37° C. incubator and additional readings were taken at 3, 6, 24, 30 and 48 hours.

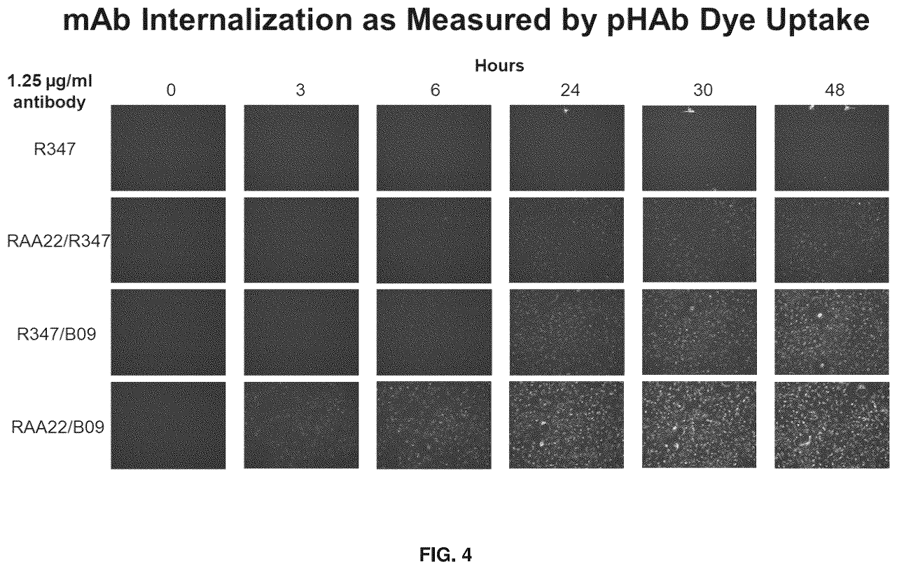

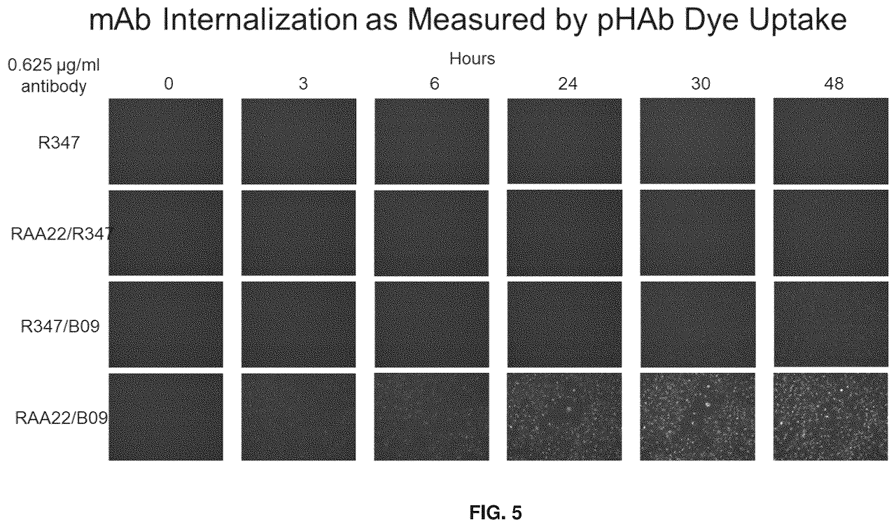

A representative internalization experiment using pHAb labeled mAbs at 1.25 μg/mL to treat NCI H1975 cells is shown in FIG. 4. The non-binding IgG1 isotype control antibody, R347, showed no detectable fluorescence at any time point. The EGFR/cMET bispecific antibody, RAA22/B09, exhibited intracellular fluorescence by 3 hours and fluorescence intensity continued to increase out to 48 hours post treatment. The monovalent EGFR binding control antibody, RAA22/R347, showed very weak fluorescence starting at 24 hours, which did not increase dramatically by 48 hours. The monovalent monospecific cMET binding control antibody, B09/R347, showed modest fluorescence at 24 hours, increasing further by 48 hours. Nevertheless, the intensity of the fluorescent signal of the monospecific cMET antibody was modest compared to the bispecific RAA22/B09, suggesting that the bispecific antibody has greater internalization efficiency than the monospecific parental antibody in the dual target expressing cell line tested here. When the cells were treated with pHAb labeled mAbs at 0.625 μg/mL, the difference between bispecific antibody and monospecific controls was even more striking (FIG. 5). The monospecific antibodies showed very little fluorescence, even at 48 hours, while the bispecific RAA22/B09 antibody again showed intracellular fluorescence by 3 hours and fluorescence intensity continued to increase out to 48 hours post treatment. These results are consistent with the hypothesis that the dual targeting bispecific antibody mediates efficient internalization into cells co-expressing both EGFR and cMET. At the same time, the monospecific parental antibodies show a reduced uptake and fluorescence intensity compared to the bispecific antibody. A logical extension of these conclusions is to suggest that the bispecific antibody might behave more like the monospecific antibodies in tissues that express only one but not both targets, which is generally true for EGFR and cMET. Of particular note, the pHAb labeled lowered affinity EGFR control mAb, RAA22/R347, exhibited negligible uptake and fluorescence. This reduced binding to EGFR could minimize the impact of the antibody conjugate in normal tissues, such as the skin, which express significant levels of EGFR but little or no cMET.

### Example 4—Monitoring Antibody Internalization Using Confocal Microscopy

Internalization kinetics of labeled DuetMabs: RAA22/B09 and QD6/B09 antibodies was assessed in vitro using live cell confocal fluorescence microscopy.

### 4.1 Materials and Methods

H1975 and HCC827 cells were from ATCC. RAA22/B09, QD6/B09, and single-arm derivatives were from MedImmune. QD6/B09 and single arm specific controls QD6/IgG and B09/IgG, with IgG Fab arms were derived from the non-specific human IgG1 NMGC were from MedImmune. RAA22/B09 and single-arm specific controls RAA22/IgG and B09/IgG derived from the non-specific human IgG1 R347, were from MedImmune. RPMI (11875-093), HEPES (15630106), sodium pyruvate (11360070), AlexaFluor® 647 (A-20186) Monoclonal Antibody Labeling Kits, Zeba™ Spin Desalting Columns (87767), and CellTracker™ Blue CMAC (C2110) were from Life Technologies (Carlsbad, CA). Accutase Cell Detachment Solution (423201) was from BioLegend (San Diego, CA). HyClone heat-inactivated fetal bovine serum (SH30071.03HI) was from GE Life Sciences (Marlborough, MA). PBS (21-040) was from Corning Incorporated (Corning, NY). FcR Blocking Reagent (130-059-901) was from Miltenyi Biotec Inc. (Auburn, CA). Polypropylene round-bottomed tubes (352063) were from BD Biosciences (San Jose, CA). CellCarrier 384-well microplates were from PerkinElmer Inc. (Cat #6007550, Waltham, MA).

### Preparation of AlexaFluor Conjugates

Monoclonal antibodies were conjugated with AlexaFluor-647 dyes using the antibody labeling kits according to manufacturer instructions. In brief, 50-100 micrograms of an antibody in the sodium bicarbonate buffer, pH=8.3, were incubated with reactive dye reagents under gentle agitation at room temperature for 1 h. Unincorporated dyes were removed by size exclusion chromatography using Zeba™ Spin Desalting Columns with 40K MWCO equilibrated with 1×PBS according to manufacturer's instructions.

### Culture and Preparation of Cells for Staining

Adherent H1975 or HCC827 cells were cultured in T-75 flasks in the CO₂ incubators using media RPMI-1640 containing 10% fetal bovine serum (FBS) to 80-90% confluency after initial seeding. On the experiment day, adherent monolayers grown in T-75 flasks were dissociated into cell suspension using Accutase. Detached cells were washed twice with 1×PBS using centrifugation at 300×g for 5 min. Cells were then resuspended into phenol-free RPMI at concentration of 2×106 cells/mL and used for staining.

### Cell Staining for Imaging

Cell suspension at 2×106 cells/mL were incubated for 30 min with 1 μM CellTracker™ Blue CMAC prepared in phenol-free RPMI at 37° C. in the CO₂ incubator. Unincorporated CellTracker™ Blue CMAC dye was removed by two washes with phenol-free RPMI using centrifugation at 300×g for 5 min at 4° C. Cells were then chilled on ice and blocked with the 10 ul FcR Blocking Reagent per 1×106 cells for 15 minutes. 2×10⁵ cells were aliquoted into 5 mL round-bottomed tubes and incubated with fluorescent antibodies at a final concentration of 2.5 μg/mL. After removal of unbound fluorescent reagents by centrifugation at 4° C., cells were resuspended in phenol-free RPMI containing 100 mM HEPES, 1 mM sodium pyruvate, and 1% FBS. Cells were transferred into multiple wells of a 384-well imaging plate at a density of 5,000 cells per well, and briefly centrifuged at 2,200 rpm for 2 min at 4° C. prior to image acquisition.

### Acquisition of Cell Images Using Confocal Fluorescence Microscopy

Stained cells in imaging plates (384-well format) were either imaged on an Opera confocal fluorescence imaging system as previous described previously (Vainshtein, 2015) or transferred onto a Zeiss Axio Observer.Z1 inverted microscope with 40×/1.2NA LCIPlan Apo objective (Carl Zeiss Microscopy, Thornwood, NY). For experiments using the Zeiss microscope, the imaging environment was kept at 37° C. at 5% CO₂ and 70% humidity using an Incubator XLmulti S DARK (PeCon Gmbh, Erbach, Germany). Samples were illuminated by 405, 488, 561, and 63 nm solid-state lasers (Carl Zeiss Microscopy, Thornwood, NY). A series of images was acquired at indicated times using a Yokogawa CSU-X1 Spinning Disk Unit (Yokogawa Electric Corporation, Tokyo, Japan) with Evolve 512 EMCCD (Photometrics, Tucson, AZ). Prior to image acquisition, exposure parameters, such as laser power, exposure times, camera gain, etc., were determined using an aliquot of the stained cells. Images were processed using ZEN 2.3 (Carl Zeiss Microscopy, Thornwood, NY) and analyzed using Columbus software (PerkinElmer, Waltham, MA).

### Algorithm for Image Analysis of Internalization

The algorithm used for quantification of antibody internalization was described previously (Vainsthein, 2015), with the following updates and modifications. The reference channel used for iterative image processing originated from the CellTracker™ Blue CMAC (CTB) staining of cells. Signal channel still derived from the antibody-AlexaFluor-647 channels of the imagers. Images were processed by the algorithm using algorithm-defined parameters, which were initially set as default values and then optimized for each cell type and experiment. CTB staining of the image was used to identify cells using thresholding to detect a region on the image having a higher intensity than its surrounding based excluding areas with fluorescence intensity signals below the threshold. The remaining identified cell objects were designated “Total Cell”. Cells were further selected filtering on morphology properties area (objects between 120-600 μm²) and roundness (>0.5). “Membrane Region” and “Cytoplasm Region” in the accepted cells were then constructed around the object boundaries using algorithm-defined parameters. Fluorescence intensity in each region was used to monitor antibody-associated AlexaFluor-647 signals. The fluorescence intensities of each region were reported as the mean of the sum of all pixels in accepted cells.

Accumulation of antibody-associated fluorescence in the cytoplasm was used to quantify kinetics of antibody internalization. To ensure comparability of results due to variability in cell staining and fluorescence intensity, cytoplasmic signals were normalized by the total cell signal at each time point and designated as the Internalized Fraction using the equation: Internalized Fraction=Intensity(cytoplasm)/(Intensity (cytoplasm)+Intensity (membrane)). Internalization rate constants kint were calculated from internalization time course by curve fitting of the data using the equation: Fcyt(t)=(1−e−kint·t)·Fmax,cyt, where Fmax,cyt is the maximal ratio cytoplasmic intensity per cell to total intensity per cell. The curve fitting of the data was conducted using Graphpad Prism (GraphPad Software, La Jolla, CA). The half-life of internalization (T½) was calculated as the ratio of ln(2) and kint.

### 4.2 In Vitro Internalization of RAA22/B09 and QD6/B09

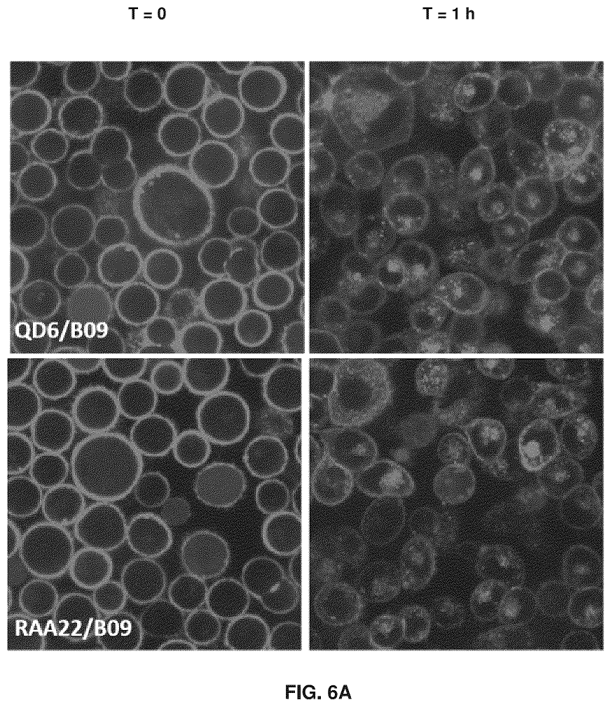

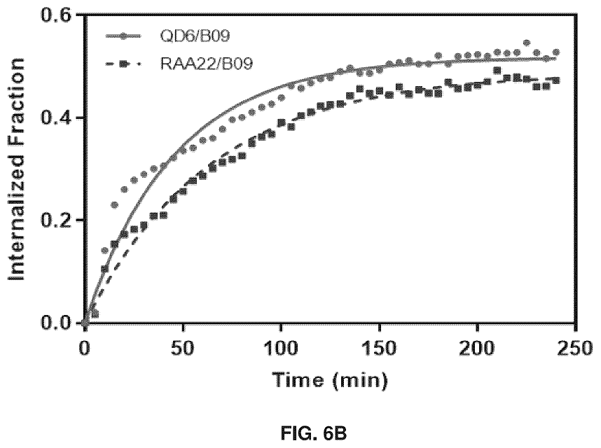

Internalization kinetics of AlexaFluor647 (AF647) primary-labeled DuetMabs: RAA22/B09 and QD6/B09 antibodies was assessed in vitro using the EGFR and c-MET expressing cell line, H1975. Each antibody was pre-bound to cells and antibody translocation from cell surface to cytoplasm was then monitored using live cell confocal fluorescence microscopy. FIG. 6 shows both antibodies localized primarily on the cell surface before (T=0) subjecting to internalization conditions and were translocated to the cytoplasm area (blue) after 1 hour (T=1 h). Kinetic images were taken every 5 min over the time course of internalization and were processed using quantitative algorithm (see above) to determine internalization kinetic constants and half-times. FIG. 6B shows very comparable internalization kinetic for QD6/B09 and RAA22/B09 with the half-times of 37.5±10.6 min and 43.2±15.5, respectively.

To evaluate the mode of antibody internalization and to investigate contribution of each arm to overall internalization of the DuetMabs, we evaluated internalization of single-arm specific control antibody molecules, QD6/IgG and B09/IgG, against duet QD6/B09, and RAA22/IgG and B09/IgG against duet RAA22/B09 in H1975 cells, which express both EGFR and c-MET. Since only one arm is specific for the target receptor, the control antibody can only internalize via one receptor eliminating dual receptor targeting and cross-linking as mode of internalization.

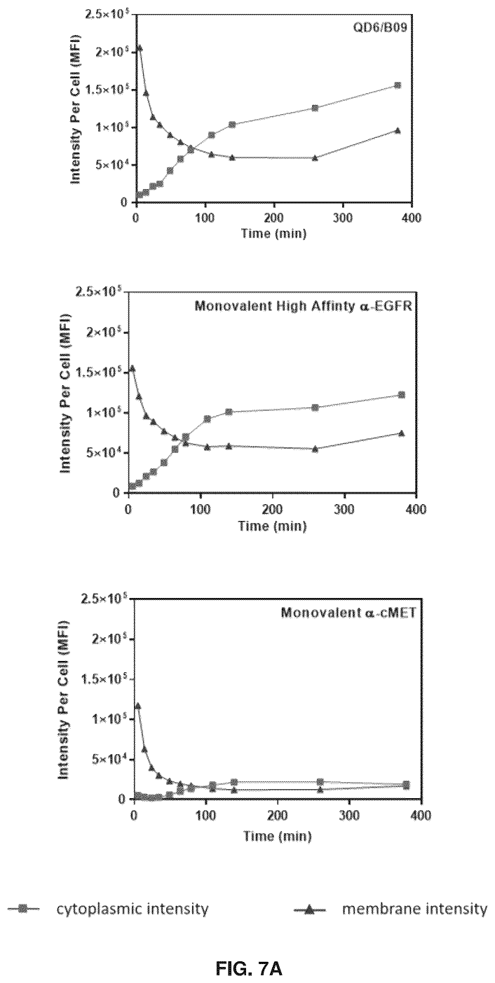

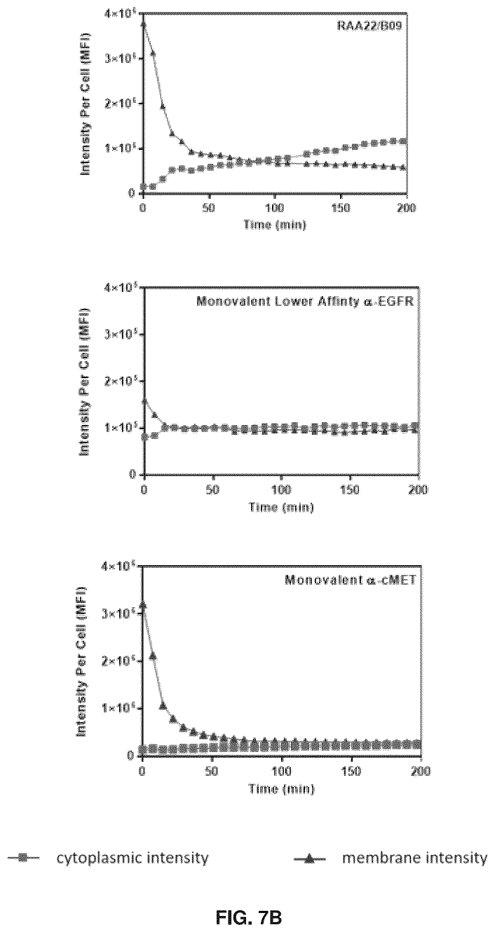

Internalization profiles of QD6/B09 (FIG. 7A) and RAA22/B09 (FIG. 7B) rendered very similar patterns of congruent decrease of membrane mAb-F1647 signals with respective increase mAb-AF647 signals in the cytoplasm, a typical profile for internalization. However, their single-arm constructs showed very different internalization profiles. Single-arm QD6/IgG had a near-identical internalization time course as the QD6/B09 DuetMab (FIG. 7A, left and middle), indicating that internalization of QD6/B09 duet was mostly driven by EGFR-arm of the molecule with minimal contribution of B09-arm. Indeed, the B09/IgG construct showed very small level of internalization (FIG. 7B, right). The rapid and extensive decrease of the membrane signal corresponded to a very moderate increase of the cytoplasm signal, likely due to extensive dissociation of pre-bound B09/IgG from c-MET receptor on the cell surface. The dissociation of the antibody subsequently resulted in modest internalization of B09/IgG. These results revealed that internalization of QD6/B09 duet was mostly driven by the EGFR-arm of the molecule with minimal contribution of the B09-arm.

In contrast, RAA22/B09 DuetMab showed internalization profiles very different when compared to its single-arm control antibodies. As seen in FIG. 7B, cytoplasmic intensity values were 10.98- and 4.70-fold higher for RAA22/B09 DuetMab than RAA22-IgG and B09-IgG, respectively. While inefficient internalization of B09/IgG maybe attributed to its pronounced dissociation (discussed above), RAA22/IgG did undergo rapid internalization. However, due to the lower affinity of the EGFR-arm, the number of RAA22/IgG molecules were 10.98-fold less (based on fluorescent intensity) than for the RAA22-B09 DuetMab. The markedly increased amount of duet RAA22/B09 mAb entering the cytoplasm as opposed to the single-arm constructs demonstrated that both antibody arms must engage with target receptors to drive internalization. This finding shows that QD6/B09 and RAA22/B09 DuetMabs have different mechanisms of internalization, with QD6/B09 primarily driven by the EGFR-arm but RAA22/B09 requiring both EGFR and c-MET arms for engagement.

4.3 Internalization of RAA22/B09 in Cell Lines with Different Levels of Target Receptors

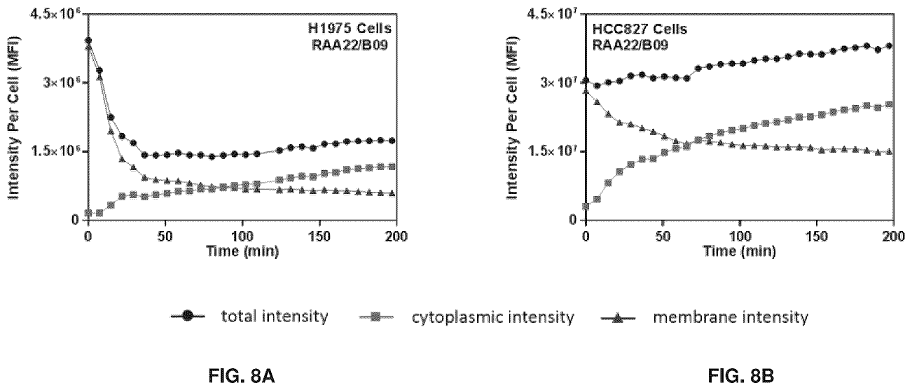

Since binding of both EGFR and c-MET arms to target receptor promoted RAA22/B09 receptor internalization in H1975 cells, we examined if the increased number of EGFR and c-MET receptors would affect internalization properties. The respective receptor levels determined by Western Blot were ˜33,000 for EGFR and ˜50,000 for c-MET in H1975 cells and ˜790,000 for EGFR and ˜523,000 for c-MET in HCC827 cells. Internalization profiles of RAA22/B09 in H1975 (medium receptor) and HCC827 (high receptor) cells shows markedly increased internalization in HCC827 cells (FIG. 8).

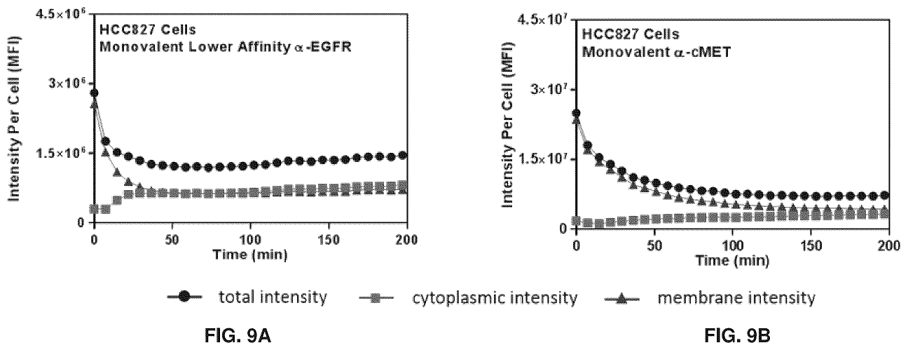

As expected in correspondence to the 23.9- fold and 10.4-fold respective increases in overall levels of EGFR and c-MET RAA22/B09, binding to HCC827 cells were on average 8.9-fold higher than H1975 (T=0, 3.1×10⁷ MFI versus 3.5×10⁶ MFI). Internalization levels (judged by peak cytoplasmic intensity) was 21.7-fold higher in HCC827 cells, suggesting that significantly higher concentrations of antibody enter the cytoplasm in cells expressing high levels of target receptors. Importantly, in addition to the markedly different intensities, the internalization profiles (membrane and cytoplasm signals over time) were also significantly distinct between HCC827 and H1975 cells. In high expressing HCC827 cells, the decrease of RAA22/B09-AF647 membrane signal corresponded to the reciprocal increase of RAA22-B09 cytoplasm signal with total RAA22/B09 signal maintained over the time course, indicating strong dual arm antibody interaction with both receptors and subsequent internalization. In H1975 cells, there was a concurrent decrease of total and membrane intensity, indicating that portions of pre-bound antibody could have dissociated from cell surface and failed to internalize inside the cell. Similar profiles indicative of dissociation were observed for internalization of RAA22/IgG and B09/IgG in HCC827 cells where single arm engagement did not render effective binding and was prone to dissociation (FIGS. 9A and 9B). This data suggested that mixed mode of receptor interaction (single arm and dual arm engagement) is present when RAA22/B09 is subjected to internalization in H1975 cells. Together, these data suggest that target cell receptor expression levels are an important determinant of the extent and efficiency of RAA22/B09 internalization.

### Example 5. General Materials and Methods for Radiopharmaceuticals

Lutetium-177 can be obtained from ITM Medical Isotopes as lutetium trichloride in a 0.05 N hydrochloric acid solution; indium-111, as indium trichloride in a 0.05 N hydrochloric acid solution, can be obtained from BWXT; and actinium-225 can be obtained as actinium-225 trinitrate from Oak Ridge National Laboratories or actinium-225 trichloride from Canadian Nuclear Laboratories.

Analytical HPLC-MS can be performed using a Waters Acquity HPLC-MS system comprised of a Waters Acquity Binary Solvent Manager, a Waters Acquity Sample Manager (samples cooled to 10° C.), a Water Acquity Column Manager (column temperature 30° C.), a Waters Acquity Photodiode Array Detector (monitoring at 254 nm and 214 nm), a Waters Acquity TQD with electrospray ionization and a Waters Acquity BEH C18, 2.1×50 (1.7 μm) column. Preparative HPLC can be performed using a Waters HPLC system comprised of a Waters 1525 Binary HPLC pump, a Waters 2489 UV/Visible Detector (monitoring at 254 nm and 214 nm) and a Waters XBridge Prep phenyl or C18 19×100 mm (5 μm) column.

HPLC elution method 1: Waters Acquity BEH C18 2.1×50 mm (1.7 μm) column; mobile phase A: H₂O (0.1% v/v TFA); mobile phase B: acetonitrile (0.1% v/v TFA); flow rate=0.3 mL/min; initial=90% A, 3-3.5 min=0% A, 4 min=90% A, 5 min=90% A.

HPLC elution method 2: Waters XBridge Prep Phenyl 19×100 mm (5 μm) column; mobile phase A: H₂O (0.1% v/v TFA); mobile phase B: acetonitrile (0.1% v/v TFA); flow rate: 10 mL/min; initial=80% A, 13 min=0% A.

HPLC elution method 3: Waters Acquity BEH C18 2.1×50 mm (1.7 μm) column; mobile phase A: H₂O (0.1% v/v TFA); mobile phase B: acetonitrile (0.1% v/v TFA); flow rate=0.3 mL/min; initial=90% A, 8 min=0% A, 10 min=0% A, 11 min=90% A, 12 min=90% A.

HPLC elution method 4: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=80% A, 3 min=80% A, 13 min=20% A, 18 min=0% A.

HPLC elution method 5: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=90% A, 3 min=90% A, 13 min=0% A, 20 min=0% A.

HPLC elution method 6: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=75% A, 13 min=0% A, 15 min=0% A.

HPLC elution method 7: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=80% A, 12 min=0% A, 15 min=0% A.

HPLC elution method 8: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=90% A, 12 min=0% A, 15 min=0% A.

Analytical Size Exclusion Chromatography (SEC) can be performed using a Waters system comprised of a Waters 1525 Binary HPLC pump, a Waters 2489 UV/Visible Detector (monitoring at 280 nm), a Bioscan Flow Count radiodetector (FC-3300) and TOSOH TSKgel G3000SWx1, 7.8×300 mm column. The isocratic SEC method can have a flow rate of, e.g., mL/min, with a mobile phase of 0.1 M phosphate, 0.6 M NaCl, 0.025% sodium azide, pH=7.

MALDI-MS (positive ion) can be performed using a MALDI Bruker Ultraflextreme Spectrometer.

Radio thin-layer chromatography (radioTLC) can be performed with Bioscan AR-2000 Imaging Scanner, and can be carried out on iTLC-SG glass microfiber chromatography paper (Agilent Technologies, SGI0001) plates using citrate buffer (0.1 M, pH 5.5).

### Example 6. Synthesis of 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B)

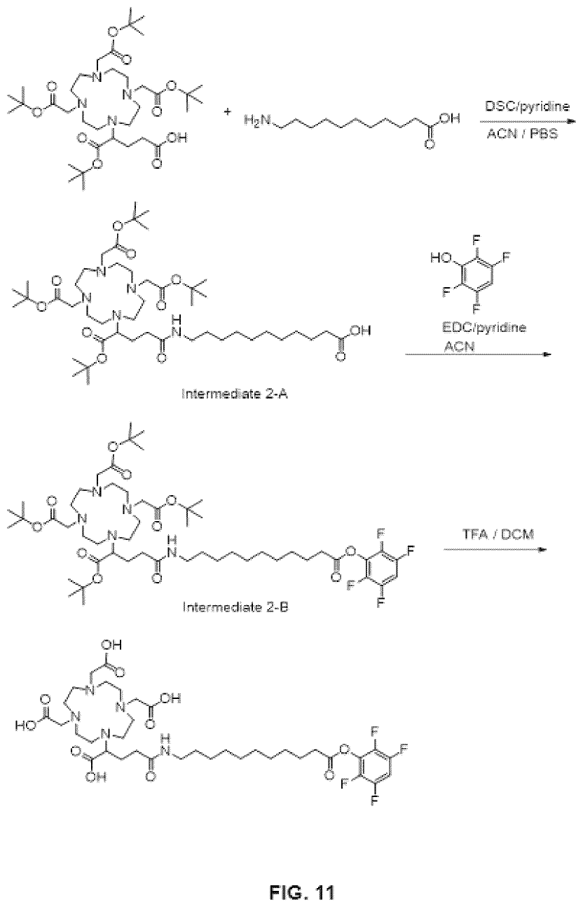

A bifunctional chelate, 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B), can be synthesized according to the scheme provided in FIG. 11. To a solution of 5-(tert-butoxy)-5-oxo-4-(4,7,10-tris(2-(tert-butoxy)-2-oxoethyl)-1,4,7,10-tetraazacyclododecan-1-yl)pentanoic acid (DOTA-GA-(tBu)₄, 50 mg, 0.07 mmol) in ACN (2.0 mL), DSC (50 mg, 0.21 mmol) is added, followed by pyridine, (0.20 mL, 2.48 mmol). The reaction is stirred at room temperature for 1 hour. To the reaction mixture is added 11-aminoundecanoic acid, (70 mg, 0.36 mmol) followed by PBS solution (1.0 mL) at room temperature. The reaction is stirred for 72 hours at room temperature. The reaction mixture is filtered with a syringe filter and purified directly by Prep-HPLC using method 6 to yield Intermediate 2-A.

To a solution of Intermediate 2-A (40 mg, 0.03 mmol), TFP (90 mg, 0.54 mmol) and EDC (40 mg, 0.27 mmol) in ACN (1.0 mL) is added pyridine (0.05 mL, 50 mg, 0.62 mmol) at room temperature. The solution is stirred at room temperature for 24 hours. The reaction is purified directly by Prep-HPLC using method 7 to provide Intermediate 2-B as a wax after concentration using a Biotage V10 Rapid Evaporator.

Intermediate 2-B is dissolved in DCM/TFA (1.0 mL/2.0 mL) and allowed to stir at room temperature for 24 hours. The reaction is concentrated by air stream and purified directly by Prep-HPLC using method 8 to yield Compound B as a clear wax after concentration. An aliquot is analyzed by HPLC-MS elution method 3.

¹H NMR (600 MHz, DMSO-d₆) δ 7.99-7.88 (m, 1H), 7.82 (t, J=5.5 Hz, 1H), 3.78 (broad s, 4H), 3.43 (broad s, 12H), 3.08 (broad s, 4H), 3.00 (m, 3H), 2.93 (broad s, 3H), 2.77 (t, J=7.2 Hz, 2H), 2.30 (broad s, 2H), 1.88 (broad s, 2H), 1.66 (p, J=7.3 Hz, 2H), 1.36 (m, 4H), 1.32-1.20 (m, 9H).

### Example 7. Synthesis of 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic acid (Compound C)

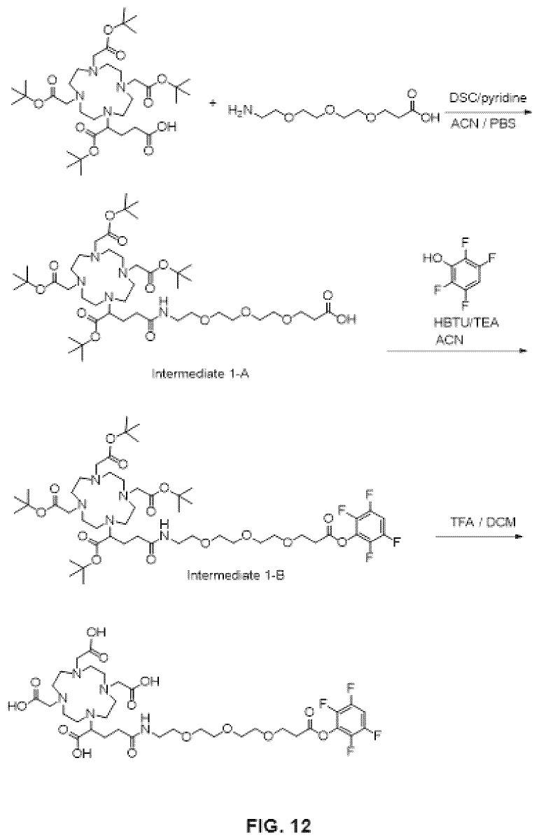

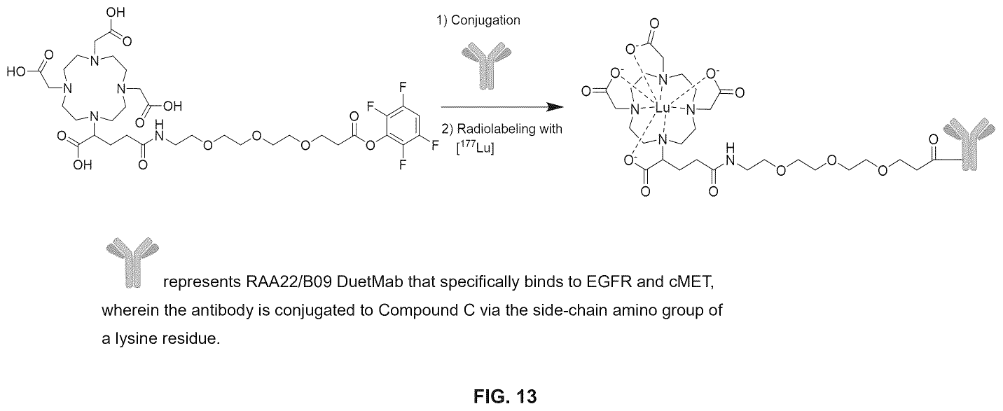

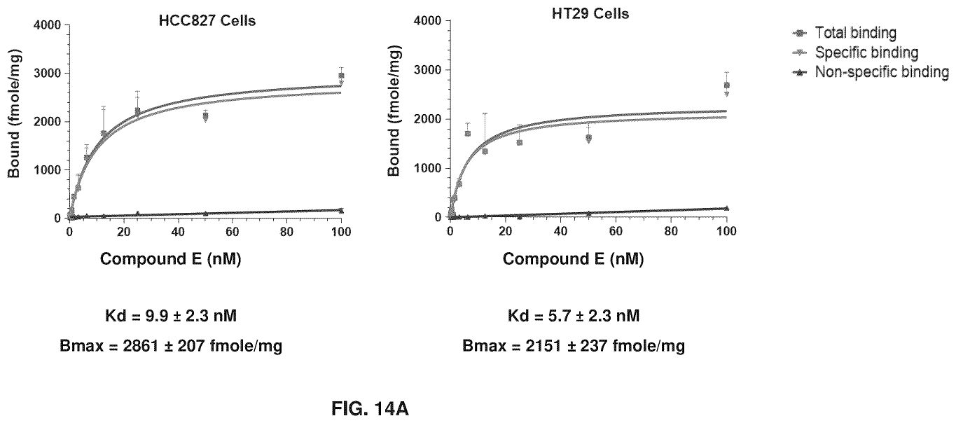

A bifunctional chelate, 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic acid (Compound C), is synthesized according to the scheme provided in FIG. 12.

To a solution of 5-(tert-butoxy)-5-oxo-4-(4,7,10-tris(2-(tert-butoxy)-2-oxoethyl)-1,4,7,10-tetraazacyclododecan-1-yl)pentanoic acid (DOTA-GA(tBu)₄, 100 mg, 0.143 mmol) in ACN (8.0 mL) is added DSC (73 mg, 0.285 mmol) and pyridine (0.80 mL, 9.89 mmol). The reaction mixture is stirred for 90 min at ambient temperature. This solution is added to a semi-solution of amino-PEG3-acid (63 mg, 0.285 mmol in 1.2 mL of DMF) in a 100 mL round bottom flask. After 4 hours at ambient temperature, the reaction is worked up by concentrating to dryness under a stream of air. The crude material is purified by HPLC elution method 2 (dissolved the crude in 6 mL of 20% ACN/H₂O). The fractions containing product are pooled and concentrated under vacuum and then co-evaporated with ACN (3×2 mL).

To a vial containing Intermediate 1-A (82 mg, 60 μmol) is added ACN (2 mL), NEt₃ (50 μL, 360 μmol, 6 equiv.), HBTU (23 mg, 60 μmol, 1 equiv) and a TFP solution (50 mg, 300 μmol, 5 equiv., dissolved in 250 μL of ACN). The resulting clear solution is stirred at ambient temperature for 3 hours. The reaction is worked up by concentrating the solution to dryness under an air stream and is then diluted with ACN/H₂O (1:1, 3 mL total) and purified on preparative HPLC using elution method 4. Fractions containing product are pooled and concentrated under vacuum and then co-evaporated with ACN (3×2 mL). Intermediate 1-B is obtained as a clear residue.

To a vial containing Intermediate 1-B (67 mg, 64 μmol) is added DCM (2 mL) and TFA (2 mL). The resulting solution is stirred at ambient temperature for 16 hour. Additional, TFA (2 mL) is added, and the reaction is stirred at ambient temperature for 6 hours. The reaction is concentrated to dryness under an air stream, with the crude product being finally dissolved in ACN/H₂O (1 mL of 10% ACN/H₂O). The crude reaction solution is then purified by preparative HPLC using elution method 5. The fractions containing product are pooled, frozen and lyophilized. Compound C is obtained as a white solid. An aliquot is analyzed by HPLC-MS elution method 3.

¹H NMR (DMSO-d₆, 600 MHz) δ 7.97-7.91 (m, 2H), 3.77 (t, 2H, J=6.0 Hz), 3.58-3.55 (m, 2H), 3.53-3.48 (m, 8H), 3.44-3.38 (m, 10H), 3.23-3.08 (m, 11H), 3.02 (t, 2H, J=6.0 Hz), 2.93 (broad s, 4H), 2.30 (broad s, 2H), 1.87 (broad s, 2H).

### Example 8. Conjugation and Radiolabeling for Synthesis of Radioimmunoconjugates Comprising EGFR-cMET Antibody

### 8.1. Synthesis of Immunoconjugate Compound D

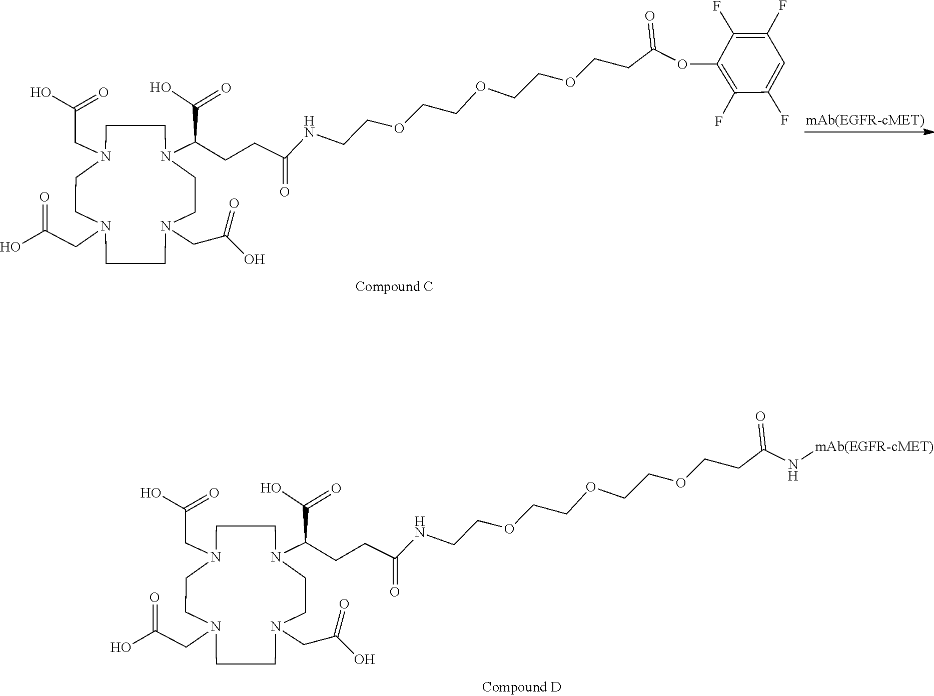

A 133 mL sterile solution container was charged with a PBS (phosphate buffered saline) solution of EGFR-cMET monoclonal bispecific antibody RAA22/B09 DuetMab (58.8 mL; 5.1 mg/mL; 300 mg), followed by 5.88 mL carbonate buffer (1.0 M, pH 9.5) and 2.83 mL of a 0.001 M HCl solution of Compound C (7.7 equiv.; 17.3 mg in 3.00 mL; this solution was prepared immediately prior to use). The pH of the resulting mixture was determined to be about 9.5-10 by pH strip. The reaction was allowed to react for two hours at room temperature. Ammonium acetate (28.1 g) was added to the reaction mixture, the resulting solution was then loaded onto a 5 mL Cytiva HiTrap Butyl HP (HIC) cartridge (the HIC cartridge was first pretreated with sodium acetate buffered saline with tween (SABST; 50 mL) followed by ammonium acetate solution (50 mL; 0.42 g/mL). The HIC cartridge was washed with the ammonium acetate solution (10 mL), then the product eluted with SABST (about 10 mL). The product solution was diluted to ca. 15 mL with SABST and loaded onto a Cytiva 15 mL HiPrep 26/10 Desalting cartridge pretreated with SABST (250 mL). The product fraction was eluted with SABST (ca. 16 mL) to afford a clear colourless liquid, wherein analysis using Size Exclusion Chromatography-High Performance Liquid Chromatography (SEC-HPLC) indicated the formation of Compound D (15.9 mL, 14.8 mg/mL, 234.9 mg, 78%). A chelate-to-antibody ratio (CAR) of 5.3 was determined using Matrix-Assisted Laser Desorption Ionization Time-of-Flight (MALDI-TOF) Mass Spectrometry, and was calculated from the difference in the mass-to charge ratio between the EGFR-cMET antibody and immunoconjugate Compound D.

### 8.2 Radiosynthesis of Radioimmunoconjugate Compound E

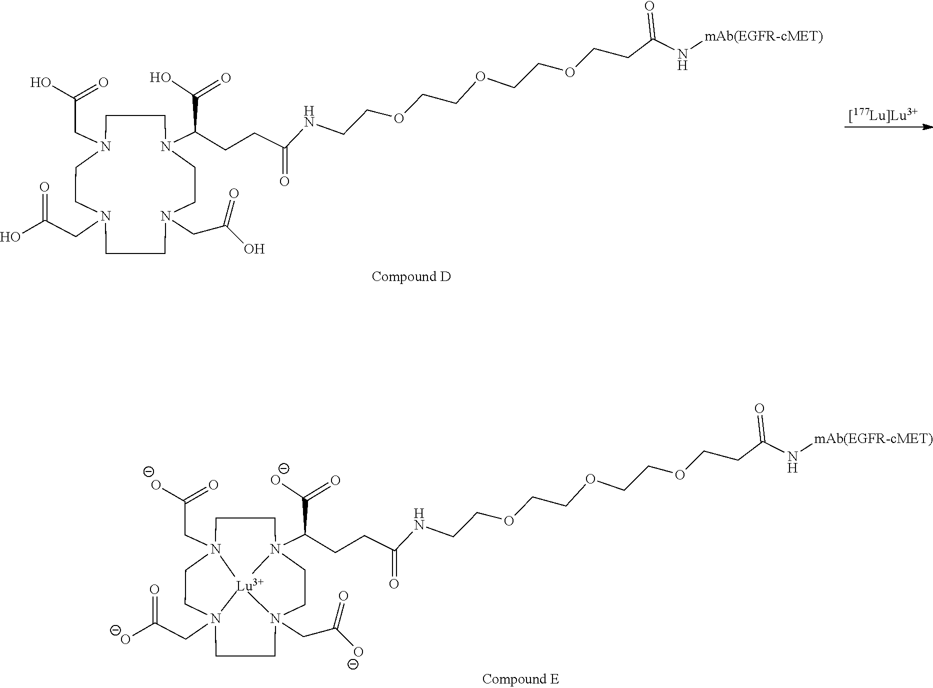

A 1.5 mL Eppendorf tube was charged with a SABST solution of Compound D (0.100 mL; 2.00 mg/mL; 0.200 mg), followed by a 0.04 M HCl solution of [¹⁷⁷Lu]LuCl₃ (2.2 mCi). After 20 min at room temperature, iTLC analysis of the reaction mixture (Silica gel (SG) plate, 0.02 M citrate buffer (5% MeOH) as the mobile phase) indicated a radiochemical conversion (RCC) of >97%. Purification was carried out using a 1 mL column packed with Sephadex G50 resin (hydrated with SABST). The product fractions were eluted using SABST and combined. SABST solutions of sodium L-ascorbate and diethylenetriaminepentaacetic acid calcium trisodium salt hydrate (DTPA) were added, and analysis of the resulting formulation (about 10 mM sodium L-ascorbate, 1 mM DTPA) by iTLC and SEC-HPLC at end-of-synthesis (EOS) indicated the formation of Compound E (364 μL, 0.431 mg/mL, 10.1 mCi/mg specific activity, >95% Radiochemical purity, and >99% chemical purity).

### 8.3 Radiosynthesis of Radioimmunoconjugate Compound F

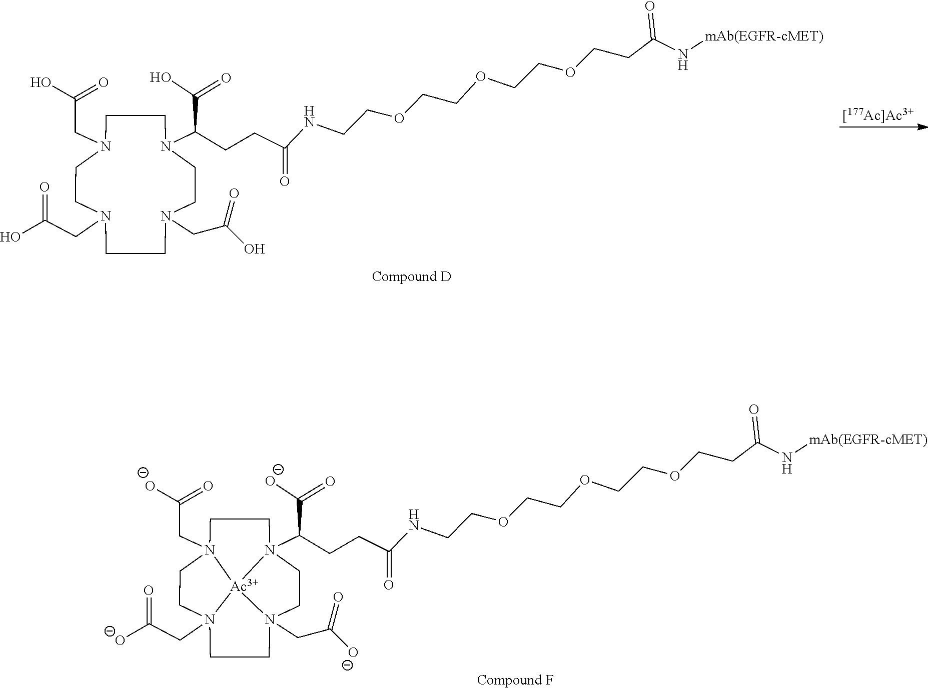

A 1.5 mL Eppendorf tube was charged with a SABST solution of Compound D (0.193 mL; 2.07 mg/mL; 0.400 mg), followed by a 0.001 M HCl solution of [²²⁵Ac]Ac(NO₃)₃ (24 μCi). After 120 min at 35° C., the reaction was quenched by the addition of DTPA (2 μL of a 0.1 M SABST solution). iTLC analysis of the reaction mixture (SG-plate, 0.02 M citrate buffer (5% MeOH) as the mobile phase) indicated a RCC of 99%. Purification was carried out using a 1 mL column packed with G50 resin (hydrated with SABST). The product fractions were eluted using SABST and combined. SABST solutions of sodium L-ascorbate and DTPA were added, and analysis of the resulting formulation (about 10 mM sodium L-ascorbate, 1 mM DTPA) by iTLC and SEC-HPLC at EOS indicated the formation of Compound F (448 μL, 0.680 mg/mL, 0.053 mCi/mg specific activity, >99% radiochemical purity, and >99% chemical purity).

### Example 9. In Vitro Binding of [¹⁷⁷Lu]-Compound C-Anti-EGFR-cMET Conjugate

A study was conducted to evaluate the receptor binding affinity of [¹⁷⁷Lu]-Compound C-anti-EGFR-cMET conjugate, i.e., Compound E, with four different cell lines: HCC827 cells, HT29 cells, H441 cells, and H1975 cells. This study followed the procedures described below.

The purpose of this assay was to ensure that the radioimmunoconjugates maintained the binding characteristics of the native antibody in EGFR and/or cMET expressing cell lines including HT29, H441, HT29, HCC827, and H1975. One day prior to the experiment, cells (1.5-2×10⁵) were seeded in 48-well microplates in 500 μL supplemented medium. At the start of the assay, cells were washed with PBS once and then treated with increasing concentrations of Compound E (0.05 nM to 100 nM) in the absence and presence of 4 μM cold antibody [total binding (TB) and non-specific binding (NSB) respectively]. Plates were incubated at 4° C. for about 3 hours with mild shaking. Following incubation, the cells were washed twice with PBS and were then lysed with 1% Triton-X-100. The lysates were transferred to gamma counting tubes and run along with Compound E standards on the Wizard 1470 gamma counter to determine the amount of radioactivity in counts per minute (CPM) for each lysate. The remaining lysate from each well (25 μL) was used for analyzing the protein content using a standard protein quantification assay.

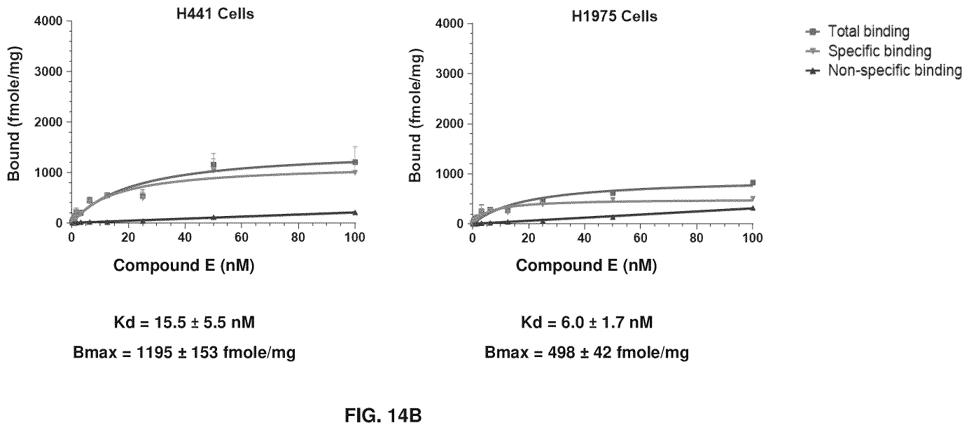

Total, specific, and non-specific binding values (fmol/mg) were plotted against conjugate concentration as shown in FIGS. 14A-14B. The Kd, and Bmax were derived by curve fitting of the specific binding data to a single-site hyperbola model (Graph Pad Prism Software, version 9). The binding data revealed desirable values for Kd. The highest Bmax (fmol/mg) was observed in HCC827 (2861±207) and HT29 (2351±237) followed by H441 (1195±153) and H1975 (498±42).

It was observed that the EGFR-cMET conjugate, i.e., Compound E, exhibited high binding affinities of about 9.9 nM with the HCC827 cells, about 5.7 nM with the HT29 cells, about 15.5 nM with the H441 cells, and about 6.0 nM with the H1975 cells.

### Example 10. Internalization Evaluation of [¹⁷⁷Lu]-Compound C-Anti-EGFR-cMET Conjugate

This internalization assay was designed to determine the degree of cell retention of radiolabeled-linker antibody derivatives. The assay relies on the inherent ability of the EGFR-cMET receptor to internalize when bound to antibody and the ability to track radiolabeled compounds. Here, a constant amount of radioimmunoconjugate is incubated with four different cell lines for a fixed duration of time and residualizations are determined by calculating the amount of internalized radioactivity as a percentage of the total cell-associated activity.

A study was conducted to evaluate the internalization of a [¹⁷⁷Lu]-Compound C-anti-EGFR-cMET conjugate, i.e., Compound E, with four different cell lines of HCC827 cells, HT29 cells, H441 cells, and H1975 cells following the protocol described below.

This assay was designed to determine the degree of cell retention of the radioimmunoconjugate Compound E. Briefly, the above-mentioned cell lines were seeded in three 24-well plates at a concentration of 3×10⁵ cells/well in complete medium (for 0 h, 2 h and 24 h incubation time). Next day, media was decanted, the cells were washed once with sterile PBS and then treated with Compound E (10 nM) for 2-3 hours at 37° C. After incubation, all the plates were immediately placed on ice and medium was discarded into pre-labeled (non-bound) gamma counting tubes. Cells were washed once with sterile PBS, gently shaken, and decanted into the (non-bound labeled) gamma tubes. Strong acid wash buffer (pH 2.5, 500 μL) was added into 0-hour time point for 5 minutes at 4° C., and then buffer was collected into pre-labeled (membrane-bound) gamma counting tubes. Cells were then lysed with 300 μL 1% Triton X-100 for 30 minutes at room temperature with gentle shaking. 250 μL of the cell lysate was transferred into gamma counting tubes and counted for 10 minutes. Mild acid wash buffer (pH 4.6, 500 μL) was added to 2-hour and 24-hour plates for 15 min at 4° C. The buffer was then collected into pre-labeled (membrane-bound) gamma counting tubes. 1 mL of warmed media was added to the plates for further incubation at 37° C. for 2 and 24 hours respectively. Following the prescribed incubation times, plates were placed on ice and processed in the following manner—media was decanted and collected into pre-labeled (efflux) gamma tubes. Plates were then washed once with 1 mL cold PBS and added into efflux tubes. Strong acid wash buffer was added to all wells and plates were incubated for 5 minutes on ice. The acid wash fraction was then collected into pre-labeled (recycled) gamma tubes. Cells were lysed with 300 uL 1% Triton X-100 for 30 minutes at room temperature. 250 μL of the cell lysate was transferred into pre-labeled (retained) gamma counting tubes and counted for 10 minutes. 25 μL of the cell lysate fraction was transferred to a 96-well plate for protein quantification (Pierce BCA Protein Assay).

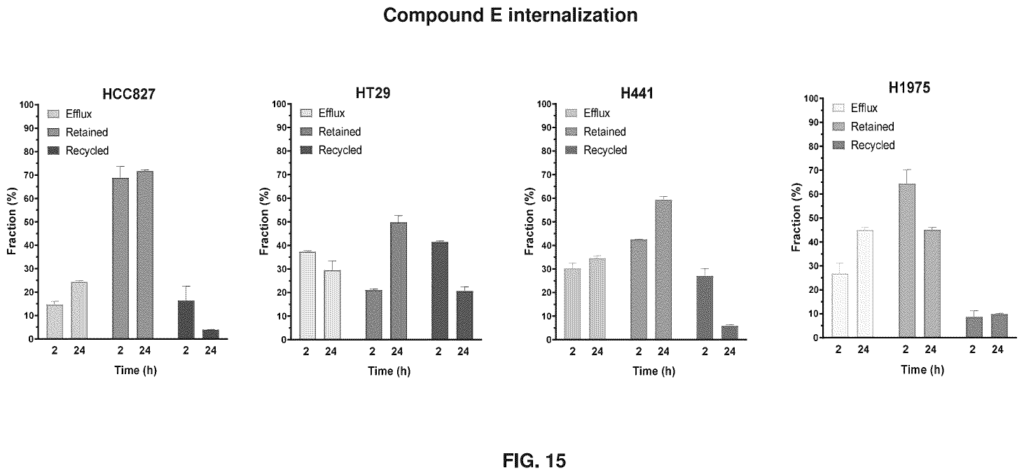

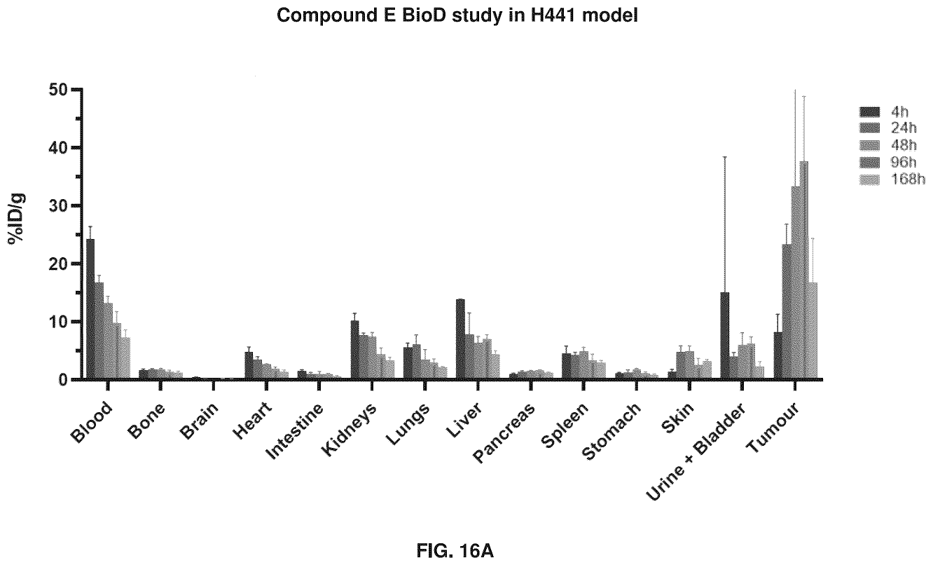

The results from the internalization study are shown in FIG. 15. Percent residualization was determined as CPM (lysate) or CPM (efflux+recycled+lysate). The lowest % efflux at 24 hours post-incubation was observed in HCC827, followed by HT29, H441 and H1975 (about 25%, 30%, 35%, and 45%, respectively). There seemed to be a correlation between the expression level EGFR+cMET receptors and cellular retention in this case.

### Example 11. In Vivo Biodistribution of [¹⁷⁷Lu]-Compound C-Anti-EGFR-cMET Conjugate in Various Animal Models

Five different cell line xenograft mouse models were used to assess the in vivo biodistribution of [¹⁷⁷Lu]-DOTA-anti-EGFR-cMET conjugate, i.e., Compound E, following the below protocol.

Tumor inoculations: Cells were washed with PBS and detached with 0.25% trypsin EDTA. The harvested cells were resuspended in a 1:1 mixture of PBS and Matrigel (BD, Oakville ON) at the following concentrations:
HCC827: 50×10⁶ cells/mL
HT29: 20×10⁶ cells/mL
H1975: 50×10⁶ cells/mL
H292: 30×10⁶ cells/mL
H441: 50×10⁶ cells/mL

5 to 7-week-old female Balb/c NCI Athymic NCr-nu/nu mice (Charles River Laboratories) were injected subcutaneously into the right flank with 100 μL of the mixture. Radioactive injections started at about 7-10 days post inoculation when tumor volume reached to 150-200 mm3, except for HCC827 xenografts that reached the same size at 3 weeks post inoculation.

Biodistribution studies: Five groups of 3 mice with subcutaneous tumors (described above) were injected intravenously via the lateral tail vein with 200 μL of Compound E containing approximately 0.74 MBq of ¹⁷⁷Lu (about 2 μg of antibody). At specified timepoints (4 h, 24 h, 48 h, 96 h, and 168 h) post injection, one group per timepoint were anesthetized with isoflurane, exsanguinated via cardiac puncture then euthanized for blood and different organ collection by dissection. Tumor and organs were rinsed with PBS of any residual blood, blotted dry and collected into pre-weighed gamma counting tubes. Radiation counts per minute contained in tissue samples were measured using a gamma counter then converted to decay corrected μCi of activity using a calibration standard. Activity measurements and sample weights were used to calculate the percent of injected dose per gram of tissue weight (% ID/g).

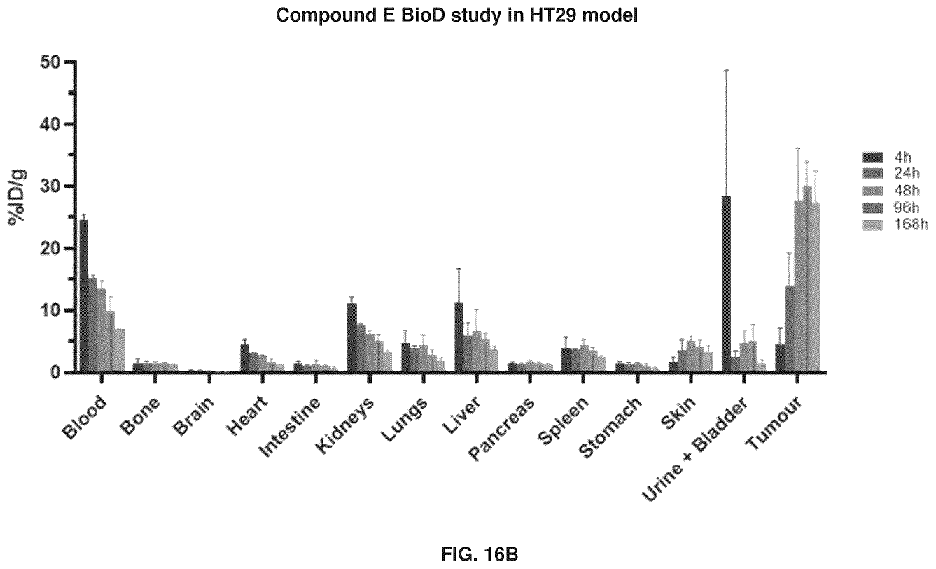

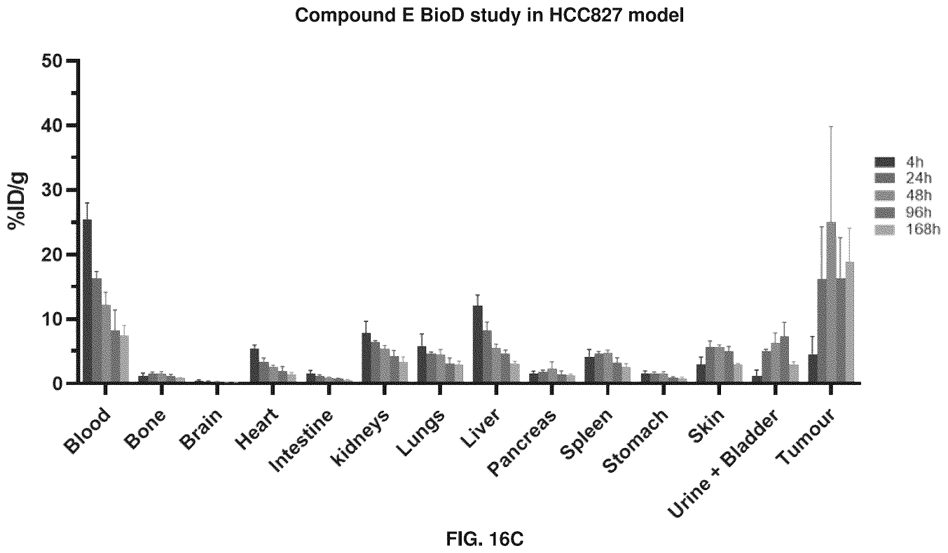

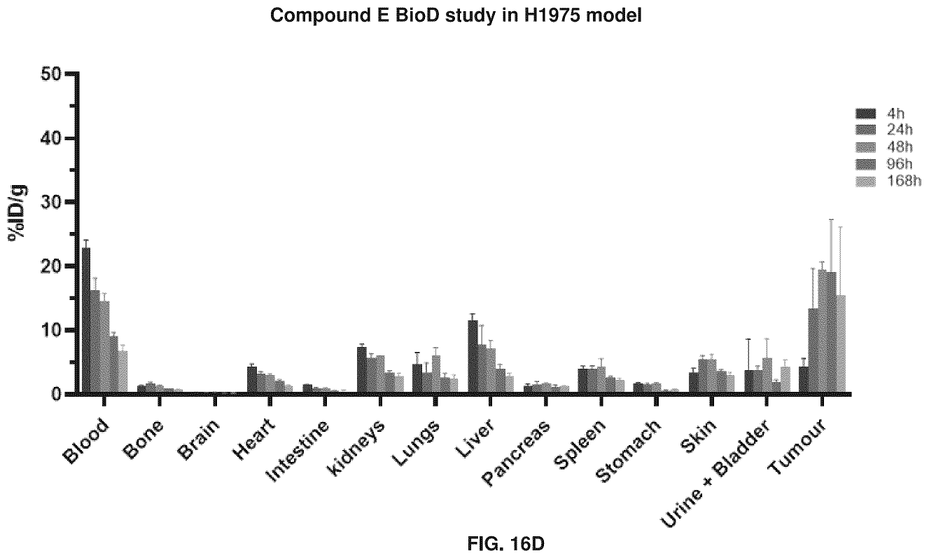

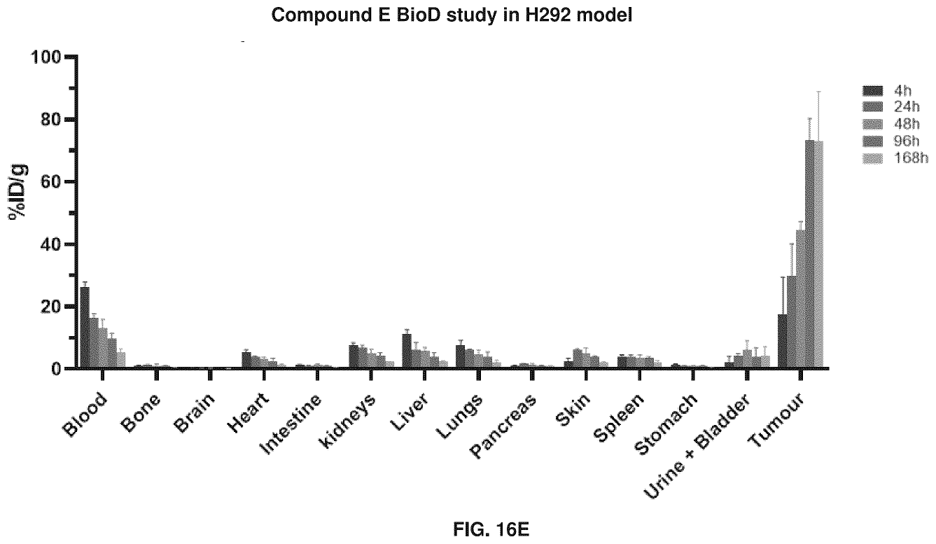

Results were expressed as the percentage injected dose per gram of tissue (% ID/g) and are depicted in FIGS. 16A-16E. Biodistribution study of Compound E showed the typical biodistribution profile for IgGs with acceptable levels of uptake in normal organs. The highest tumor uptake (% ID/g) was observed in H292>H441>HT29 and H1975 xenografts [about 75% (96 h, 168 h), about 35% (48 h, 96 h), about 29% (48 h, 96 h), and about 20% (48 h, 96 h), respectively]. Tumor uptake (% ID/g) peaked at 48 h with about 25% Compound E in HCC827 xenograft model and declined to about 17% at 96 h, and 168 h post injection. HCC827 xenograft model was not pursued for therapy study due to slow tumor growth rate.

### Example 12. In Vivo Efficacy of [²²⁵Ac]-Compound C-Anti-EGFR-cMET Conjugate in Various Animal Models

A study was designed to evaluate the efficacy for different doses of actinium-225 labeled radioimmunoconjugate, Compound F, as compared to the cold antibody clone and/or vehicle control.

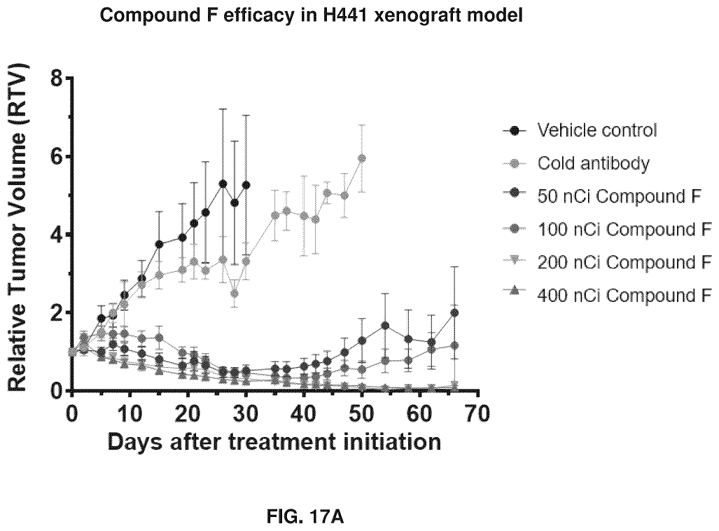

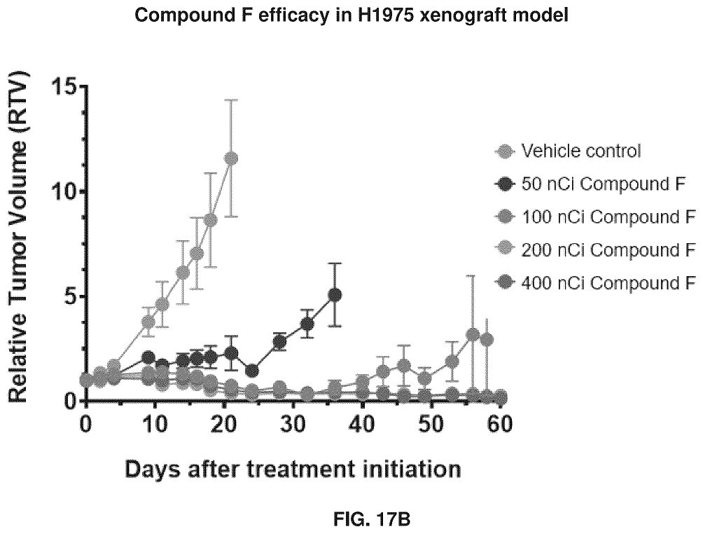

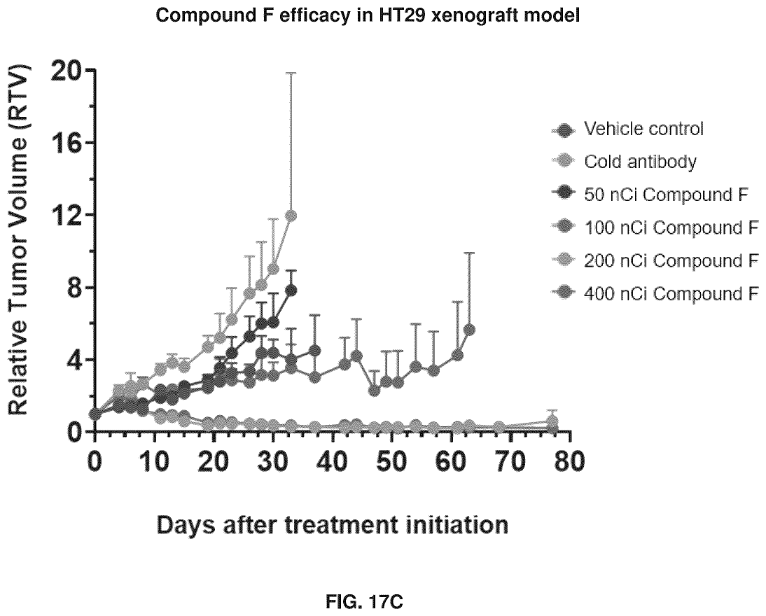

The Efficacy study was done with four increasing doses of Compound F (²²⁵Ac radiolabeled EGFR-cMET antibody) and compared to the cold-antibody and/or vehicle control. Therapeutic efficacy studies were carried out using H441, HT29 and H1975 tumor xenografts. For each study, 6-7 groups of tumor-bearing animals (n=5) were injected intravenously via the lateral tail vein with 200 μL of compounds. Compound F was dosed at 50-400 nanocuries (nCi) of activity formulated in 20 mM sodium citrate pH 5.5, 0.82% NaCl, and 0.01% Tween-80 buffer. As a control, non-radiolabeled, non-conjugated antibody was administered at a protein mass equivalent corresponding to the highest radioactivity dose of Compound F tested in a study. Tumor measurements were taken 2-3 times per week for at least 60 days with vernier calipers in two dimensions. Tumor length was defined as the longest dimension, width was measured perpendicular to the tumor length. At the same time animals were weighed. Overall body condition and general behavior were assessed daily. Tumor volume (mm) was calculated from caliper measurements as an ellipsoid: Tumor growth was expressed as relative tumor volume (RTV) which is tumor volume measured on day X divided by the tumor volume measured on the day of dosing. In all the models, 200 nCi and 400 nCi caused long term tumor regression in all the mice (FIGS. 17A-17C). The best response was observed in the H441 model with both 50 nCi and 100 nCi doses showing tumor suppression and regression in several mice. In the H1975 model, 50 nCi significantly delayed tumor growth in all the mice, and 100 nCi-treated group showed a mixed response including delayed tumor growth, tumor suppression and regression. In the HT29 model, no efficacy was observed with 50 nCi, or 100 nCi doses, except for one mouse that showed tumor suppression with 100 nCi.

### REFERENCES

A number of publications are cited above in order to more fully describe and disclose the disclosure and the state of the art to which the disclosure pertains. Full citations for these references are provided below. The entirety of each of these references is incorporated herein.

| | |
|---|---|
| Ariyawutyakorn, | Ariyawutyakorn, W., S. Saichaemchan, and M. Varella-Garcia, |
| 2016 | Understanding and Targeting MET Signaling in Solid Tumors - Are We There |
| | Yet? Journal of Cancer, 2016. 7(6): p. 633-649. |
| Arteaga, 2014 | Arteaga, C. L. and J. A. Engelman, ERBB receptors: from oncogene discovery |
| | to basic science to mechanism-based cancer therapeutics. Cancer Cell, 2014. |
| | 25(3): p. 282-303. |
| Bachleitner- | Bachleitner-Hofmann, T., et al., HER kinase activation confers resistance to |
| Hofmann, 2008 | MET tyrosine kinase inhibition in MET oncogene-addicted gastric cancer |
| | cells. Mol Cancer Ther, 2008. 7: p. 3499-3508. |
| Bardelli, 2013 | Bardelli, A., et al., Amplification of the MET Receptor Drives Resistance to |
| | Anti-EGFR Therapies in Colorectal Cancer. Cancer Discovery, 2013. 3(6): p. |
| | 658-673. |
| Bean, 2007 | Bean, J., et al., MET amplification occurs with or without T790M mutations in |
| | EGFR mutant lung tumors with acquired resistance to gefitinib or erlotinib. |
| | Proceedings of the National Academy of Sciences of the United States of |
| | America, 2007. 104(52): p. 20932-20937. |
| Belalcazar, 2010 | Belalcazar, A., et al., Targeting the Met pathway in lung cancer. Expert |
| | review of anticancer therapy, 2012. 12(4): p. 519-528. |
| Benedettini, | Benedettini, E., et al., Met activation in non-small cell lung cancer is |
| 2010 | associated with de novo resistance to EGFR inhibitors and the development of |
| | brain metastasis. The American journal of pathology, 2010. 177(1): p. 415- |
| | 423. |
| Bertotti, 2015 | Bertotti, A. and F. Sassi, Molecular Pathways: Sensitivity and Resistance to |
| | Anti-EGFR Antibodies. Clinical Cancer Research, 2015. |
| Birchmeier, | Birchmeier, C., et al., Met, metastasis, motility and more. Nat Rev Mol Cell |
| 2003 | Biol, 2003. 4: p. 915-925. |
| Boccaccio, 2014 | Boccaccio, C., P. Luraghi, and P. M. Comoglio, MET-Mediated Resistance to |
| | EGFR Inhibitors: An Old Liaison Rooted in Colorectal Cancer Stem Cells. |
| | 2014. p. 3647-3651. |
| Bouattour, 2018 | Bouattour, M., et al., Recent developments of c-Met as a therapeutic target in |
| | hepatocellular carcinoma. Hepatology (Baltimore, Md.), 2018. 67(3): p. |
| | 1132-1149. |
| Cappuzzo, 2009 | Cappuzzo, F., et al., Increased MET gene copy number negatively affects |
| | survival of surgically resected non-small-cell lung cancer patients. J Clin |
| | Oncol, 2009. 27(10): p. 1667-74. |
| Chan, 2017 | Chan, D. L. H., et al., Epidermal growth factor receptor(EGFR)inhibitors for |
| | metastatic colorectal cancer. Cochrane Database Syst Rev, 2017. 6: p. |
| | CD007047. |
| Chong, 2013 | Chong, C. R. and P. A. Janne, The quest to overcome resistance to EGFR- |
| | targeted therapies in cancer. Nature medicine, 2013. 19(11): p. 1389-1400. |
| Chothia, 1987 | Chothia, C. and Lesk, A. M., Canonical structures for the hypervariable |
| | regions of immunoglobulins. J. Mol. Biol. 1987, 196: 901-917 |
| Choueiri, 2017 | Choueiri, T. K., et al., Biomarker-Based Phase II Trial of Savolitinib in |
| | Patients With Advanced Papillary Renal Cell Cancer. J Clin Oncol, 2017. |
| | 35(26): p. 2993-3001. |
| Corso, 2010 | Corso, S., et al., Activation of HER family members in gastric carcinoma cells |
| | mediates resistance to MET inhibition. Molecular Cancer, 2010. 9(1): p. 121. |
| Cunningham, | Cunningham, D., et al., Cetuximab monotherapy and cetuximab plus |
| 2004 | irinotecan in irinotecan-refractory metastatic colorectal cancer. N Engl J |
| | Med, 2004. 351(4): p. 337-45. |
| De Bacco, 2012 | De Bacco, F., et al., The MET Oncogene Is a Functional Marker of a |
| | Glioblastoma Stem Cell Subtype. Cancer research, 2012. 72(17): p. 4537- |
| | 4550. |
| Dimasi, 2017 | Dimasi et al. Efficient Preparation of Site-Specific Antibody-Drug Conjugates |
| | Using Cysteine Insertion. Mol Pharmaceuticals, 2017, 14(5) 1501-1516 |
| Dokala, 2005 | Dokala, A. and S. S. Thakur, Extracellular region of epidermal growth factor |
| | receptor: a potential target for anti-EGFR drug discovery. Oncogene, 2016. |
| | 36: p. 2337. |
| Engelman, 2007 | Engelman, J. A., et al., MET amplification leads to gefitinib resistance in lung |
| | cancer by activating ERBB3 signaling. Science, 2007. 316: p. 1039-1043. |
| Garber, 2014 | Garber, K., MET inhibitors start on road to recovery. Nature reviews. Drug |
| | discovery, 2014. 13(8): p. 563-565. |
| Gherardi, 2013 | Gherardi et al., Functional map and domain structure of MET, the product of |
| | the c-met protooncogene and receptor for hepatocyte growth factor/scatter |
| | factor. Proc Natl Acad Sci U.S.A, 2003, 100(21): 12039-12044 |
| Giordano, 1989 | Giordano, S., et al., Tyrosine kinase receptor indistinguishable from the c-met |
| | protein. Nature, 1989. 339: p. 155-156. |
| Gisterek, 2011 | Gisterek, I., et al., Prognostic role of c-met expression in breast cancer |
| | patients. Rep Pract Oncol Radiother, 2011. 16(5): p. 173-7. |
| Gou, 2016 | Gou, L. Y., et al., The coexistence of MET over-expression and an EGFR |
| | T790M mutation is related to acquired resistance to EGFR tyrosine kinase |
| | inhibitors in advanced non-small cell lung cancer. Oncotarget, 2016. |
| Guo, 2008 | Guo, A., et al., Signaling networks assembled by oncogenic EGFR and c-Met. |
| | Proceedings of the National Academy of Sciences, 2008. 105(2): p. 692-697. |
| Guo, 2014 | Guo, B., et al., Prognostic value of MET gene copy number and protein |
| | expression in patients with surgically resected non-small cell lung cancer: a |
| | meta-analysis of published literatures. PLoS One, 2014. 9(6): p. e99399. |
| Haura, 2013 | Haura, E. B. and M. A. and Smith, Signaling Control by Epidermal Growth |
| | Factor Receptor and MET: Rationale for Cotargeting Strategies in Lung |
| | Cancer. Journal of Clinical Oncology, 2013. 31(32): p. 4148. |
| Hay, 2016 | Hay et al., Targeting CD73 in the tumor microenvironment with MEDI9447. |
| | Oncoimmunology, 2016 5(8): e1208875. |
| Huang, 2014 | Huang, L., et al., MET expression plays differing roles in non-small-cell lung |
| | cancer patients with or without EGFR mutation. Journal of thoracic oncology: |
| | official publication of the International Association for the Study of Lung |
| | Cancer, 2014. 9(5): p. 725-728. |
| Jimeno, 2009 | Jimeno, A., et al., KRAS mutations and sensitivity to epidermal growth factor |
| | receptor inhibitors in colorectal cancer: practical application of patient |
| | selection. J Clin Oncol, 2009. 27(7): p. 1130-6. |
| Jun, 2013 | Jun, H. J., R. T. Bronson, and A. Charest, Inhibition of EGFR induces a c-MET |
| | driven stem cell population in Glioblastoma. Stem cells (Dayton, Ohio), 2013. |
| Kabat, 1991 | Kabat, E. A. et al. Sequences of Proteins of Immunological Interest, 5th Ed. |
| | Public Health Service, National Institutes of Health, Bethesda, MD, 1991 |
| Karamouzis, | Karamouzis, M. V., P. A. Konstantinopoulos, and A. G. Papavassiliou, |
| 2009 | Targeting MET as a strategy to overcome crosstalk-related resistance to |
| | EGFR inhibitors. The Lancet Oncology, 2009. 10(7): p. 709-717. |
| Kim, 2017a | Kim, J. H., B. J. Kim, and H. S. Kim, Clinicopathological impacts of high c- |
| | Met expression in head and neck squamous cell carcinoma: a meta-analysis |
| | and review. Oncotarget, 2017. 8(68): p. 113120-113128. |
| Kim, 2017b | Kim, J. H., B. J. Kim, and H. S. Kim, Clinicopathological impacts of high c- |
| | Met expression in renal cell carcinoma: a meta-analysis and review. |
| | Oncotarget, 2017. 8(43): p. 75478-75487. |
| Kim, 2017c | Kim, J. H., et al., Prognostic value of c-Met overexpression in hepatocellular |
| | carcinoma: a meta-analysis and review. Oncotarget, 2017. 8(52): p. 90351- |
| | 90357. |
| Klein, 2012 | Klein et al. Progress in overcoming the chain association issue in bispecific |
| | heterodimeric IgG antibodies. MAbs, 2012, 4: 653-63 |
| Knickelbein, | Knickelbein, K. and L. Zhang, Mutant KRAS as a critical determinant of the |
| 2015 | therapeutic response of colorectal cancer. Genes Dis, 2015. 2(1): p. 4-12. |
| Kondo, 2013 | Kondo, S., et al., Clinical impact of c-Met expression and its gene |
| | amplification in hepatocellular carcinoma. International journal of clinical |
| | oncology, 2013. 18(2): p. 207-213. |
| Lacouture, 2006 | Lacouture, M. E., Mechanisms of cutaneous toxicities to EGFR inhibitors. |
| | Nature reviews. Cancer, 2006. 6(10): p. 803-812. |
| LeFranc, 2015 | LeFranc, M. P. et al., IMGT ®, the international ImMunoGene Tics information |
| | system ® 25 years on., Nucleic Acids Res. 2015, 43(Database issue): D413-22. |
| Li, 2011 | Li, C., et al., c-Met Is a Marker of Pancreatic Cancer Stem Cells and |
| | Therapeutic Target. Gastroenterology, 2011. 141(6): p. 2218-2227. e5. |
| Liska, 2011 | Liska, D., et al., HGF Rescues Colorectal Cancer Cells from EGFR Inhibition |
| | via MET Activation. Clinical Cancer Research, 2011. 17(3): p. 472-482. |
| Liu, 2015 | Liu, Y., et al., Prognostic value of c-Met in colorectal cancer: a meta- |
| | analysis. World J Gastroenterol, 2015. 21(12): p. 3706-10. |
| Luraghi, 2014 | Luraghi, P., et al., MET Signaling in Colon Cancer Stem-like Cells Blunts the |
| | Therapeutic Response to EGFR Inhibitors. Cancer research, 2014. 74(6): p. |
| | 1857-1869. |
| Madoz-Gúrpide, | Madoz-Gúrpide, J., et al., Activation of MET pathway predicts poor outcome |
| 2016 | to cetuximab in patients with recurrent or metastatic head and neck cancer. |
| | 2016. p. 1-13. |
| Marano, 2015 | Marano, L., et al., c-Met targeting in advanced gastric cancer: An open |
| | challenge. Cancer letters, 2015. 365(1): p. 30-36. |
| Mazor, 2017 | Mazor, Y., et al., Enhanced tumor-targeting selectivity by modulating |
| | bispecific antibody binding affinity and format valence. Sci Rep, 2017. 7: p. |
| | 40098. |
| Mazor, 2015 | Mazor, Y., et al., Insights into the molecular basis of a bispecific antibody's |
| | target selectivity. MAbs, 2015. 7(3): p. 461-9. |
| McDermott, | McDermott, U., et al., Acquired Resistance of Non-Small Cell Lung Cancer |
| 2010 | Cells to MET Kinase Inhibition Is Mediated by a Switch to Epidermal Growth |
| | Factor Receptor Dependency. Cancer research, 2010. 70(4): p. 1625-1634. |
| Miyamoto, 2011 | Miyamoto, M., et al., Prognostic significance of overexpression of c-Met |
| | oncoprotein in cholangiocarcinoma. British journal of cancer, 2011. 105(1): |
| | p. 131-138. |
| Mo, 2017 | Mo, H. N. and P. Liu, Targeting MET in cancer therapy. Chronic Dis Transl |
| | Med, 2017. 3(3): p. 148-153. |
| Moores, 2016 | Moores, S. L., et al., A Novel Bispecific Antibody Targeting EGFR and cMet |
| | that is Effective Against EGFR Inhibitor-Resistant Lung Tumors. Cancer |
| | research, 2016. |
| Neuberger, 1988 | Neuberger et al., 1988, 8th International Biotechnology Symposium Part 2, |
| | 792-799 |
| Nicholson, 2001 | Nicholson, R. I., J. M. Gee, and M. E. Harper, EGFR and cancer prognosis. Eur |
| | J Cancer, 2001. 37: p. 9-15. |
| Organ, 2011 | Organ, S. L. and M. S. Tsao, An overview of the c-MET signaling pathway. |
| | Therapeutic advances in medical oncology, 2011. 3(1 Suppl): p. S7-S19. |
| Organesyan, | Organesyan et al. Structural characterization of a human Fc fragment |
| 2008 | engineered for lack of effector functions. Acta Crystallogr D Biol Crystallogr. |
| | 2008; 64(Pt 6): 700-4. |
| Ou, 2016 | Ou, S.-H. I., N. Agarwal, and S. M. Ali, High MET amplification level as a |
| | resistance mechanism to osimertinib(AZD9291)in a patient that |
| | symptomatically responded to crizotinib treatment post-osimertinib |
| | progression. Lung Cancer, 2016. 98(Supplement C): p. 59-61. |
| Persic, 1997 | Persic et al. An integrated vector system for the eukaryotic expression of |
| | antibodies or their fragments after selection from phage display libraries. |
| | Gene, 1997, 187: 9-18. |
| Peters, 2012 | Peters, S. and A. A. Adjei, MET: a promising anticancer therapeutic target. |
| | Nature reviews. Clinical oncology, 2012. 9(6): p. 314-326. |
| Prat, 1991 | Prat, M., et al., The receptor encoded by the human c-MET oncogene is |
| | expressed in hepatocytes, epithelial cells and solid tumors. International |
| | journal of cancer. Journal international du cancer, 1991. 49(3): p. 323-328. |
| Puri, 2008 | Puri, N. and R. Salgia, Synergism of EGFR and c-Met pathways, cross-talk |
| | and inhibition, in non-small cell lung cancer. J Carcinog, 2008. 7: p. 9. |
| Remon, 2018 | Remon, J., et al., Osimertinib and other third-generation EGFR TKI in |
| | EGFR-mutant NSCLC patients. Ann Oncol, 2018. 29(suppl_1): p. i20-i27. |
| Retter, 2005 | Retter, I., et al., “VBASE2, an integrative V gene database. ” Nucl. Acids Res. |
| | 2005, 33 (suppl 1): D671-D674 |
| Rho, 2009 | Rho, J. K., et al., The role of MET activation in determining the sensitivity to |
| | epidermal growth factor receptor tyrosine kinase inhibitors. Molecular cancer |
| | research: MCR, 2009. 7(10): p. 1736-1743. |
| Rocha-Lima, | Rocha-Lima, C. M., et al., EGFR targeting of solid tumors. Cancer control: |
| 2007 | journal of the Moffitt Cancer Center, 2007. 14(3): p. 295-304. |
| Sacco, 2015 | Sacco, J. J. and M. J. Clague, Dysregulation of the Met pathway in non-small |
| | cell lung cancer: implications for drug targeting and resistance. Transl Lung |
| | Cancer Res, 2015. 4(3): p. 242-52. |
| Saltz, 2004 | Saltz, L. B., et al., Phase II trial of cetuximab in patients with refractory |
| | colorectal cancer that expresses the epidermal growth factor receptor. J Clin |
| | Oncol, 2004. 22(7): p. 1201-8. |
| Seshacharyulu, | Seshacharyulu, P., et al., Targeting the EGFR signaling pathway in cancer |
| 2012 | therapy. Expert Opinion on Therapeutic Targets, 2012. 16(1): p. 15-31. |
| Sierra, 2011 | Sierra, J. R. and M.-S. Tsao, c-MET as a potential therapeutic target and |
| | biomarker in cancer. Therapeutic Advances in Medical Oncology, 2011. 3(1 |
| | suppl): p. S21-S35. |
| Sohn, 2014 | Sohn, J., et al., cMET Activation and EGFR-Directed Therapy Resistance in |
| | Triple-Negative Breast Cancer. Journal of Cancer, 2014. 5(9): p. 745-753. |
| Suda, 2010 | Suda, K., et al., Reciprocal and complementary role of MET amplification |
| | and EGFR T790M mutation in acquired resistance to kinase inhibitors in lung |
| | cancer. Clinical cancer research: an official journal of the American |
| | Association for Cancer Research, 2010. 16(22): p. 5489-5498. |
| Troiani, 2016 | Troiani, T., et al., Therapeutic value of EGFR inhibition in CRC and NSCLC: |
| | 15 years of clinical evidence. ESMO open, 2016. 1(5): p. e000088. |
| Trusolino, 2010 | Trusolino, L., A. Bertotti, and P. Comoglio, MET signalling: principles and |
| | functions in development, organ regeneration and cancer. Nature |
| | reviews. Molecular cell biology, 2010. 11(12): p. 834. |
| Vainshtein, | Vainshtein et al., Quantitative measurement of the target-mediated |
| 2015 | internalization kinetics of biopharmaceuticals, Pharm Res., 2015. 32: 286-299. |
| Vaughan, 1996 | Vaughan, et al., Human antibodies with sub-nanomolar affinities isolated |
| | from a large non-immunized phage display library, Nat Biotechnol,. 1996. 14: |
| | p. 309 |
| Vecchione, 2011 | Vecchione, L., et al., EGFR-targeted therapy. Special Issue- |
| | Gastroenterology, 2011. 317(19): p. 2765-2771. |
| Vsiansky, 2018 | Vsiansky, V., et al., Prognostic role of c-Met in head and neck squamous cell |
| | cancer tissues: a meta-analysis. Scientific Reports, 2018. 8(1): p. 10370. |
| Wang, 2018 | Wang, X. et al., IgG Fc engineering to modulate antibody effector functions |
| | Protein Cell. 2018;9(1): 63-73 |
| Wu, 2017 | Wu, Y.-L., et al., Does c-Met remain a rational target for therapy in patients |
| | with EGFR TKI-resistant non-small cell lung cancer? Cancer Treatment |
| | Reviews, 2017. 61: p. 70-81. |
| Xu, 2016 | Xu, Y. P., et al., C-Met as a Molecular Marker for Esophageal Squamous Cell |
| | Carcinoma and Its Association with Clinical Outcome. Journal of Cancer, |
| | 2016. 7(5): p. 587-594. |
| Yan, 2015 | Yan, S., et al., Prognostic significance of c-Met in breast cancer: a meta- |
| | analysis of 6010 cases. Diagn Pathol, 2015. 10: p. 62. |
| Zhang, 2016 | Zhang, Y., Z. Du, and M. Zhang, Biomarker development in MET-targeted |
| | therapy. Oncotarget, 2016. 7(24): p. 37370-37389. |
| Zhang, 2015 | Zhang, Y. W., Promise and challenges on the horizon of MET-targeted cancer |
| | therapeutics. World J Biol Chem, 2015. 6(2): p. 16-27. |
| Zucali, 2008 | Zucali, P. A., et al., Role of cMET expression in non-small-cell lung cancer |
| | patients treated with EGFR tyrosine kinase inhibitors. Annals of Oncology, |
| | 2008. 19(9): p. 1605-1612. |

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

What is claimed is:

1. A compound comprising the structure of Formula I, or a pharmaceutically acceptable salt thereof:
A-L¹-(L²)ₙ-B Formula I
wherein
A is a chelating moiety or a metal complex thereof;
B is an antibody or antigen-binding fragment thereof,
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
L² each independently has the structure of Formula II:
—X¹-L³-Z¹ Formula II
wherein
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—, or —NR¹—, wherein “*” indicates the attachment point to L³, and R¹ is hydrogen, optionally substituted C₁-C₆ alkyl, optionally substituted C₁-C₆ heteroalkyl, optionally substituted aryl, or optionally substituted heteroaryl;
L³ is optionally substituted C₁-C₅₀ alkyl or optionally substituted C₁-C₅₀ heteroalkyl; and
Z¹ is —CH₂-#, —C(O)-#, —C(S)-#, —OC(O)-#, —C(O)O-#, —NR²C(O)-#, —C(O)NR²-#, or —NR²-#, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl, and
wherein the antibody or antigen-binding fragment thereof comprises a first antigen-binding domain that is capable of binding epidermal growth factor receptor (EGFR) and a second antigen-binding domain that is capable of binding cMET,
wherein the first antigen-binding domain comprises:
i. a heavy chain variable (VH) region comprising the following complementarity determining regions (CDRs):
HCDR1 having the amino acid sequence of SEQ ID NO: 1
HCDR2 having the amino acid sequence of SEQ ID NO: 2
HCDR3 having the amino acid sequence of SEQ ID NO: 3,
or a variant thereof in which one or two or three amino acids in one or more of HCDR1, HCDR2, and HCDR3 are substituted with another amino acid; and
ii. a light chain variable (VL) region comprising the following CDRs:
LCDR1 having the amino acid sequence of SEQ ID NO: 4
LCDR2 having the amino acid sequence of SEQ ID NO: 5
LCDR3 having the amino acid sequence of SEQ ID NO: 6,
or a variant thereof in which one or two or three amino acids in one or more of LCDR1, LCDR2, and LCDR3 are substituted with another amino acid.

2. The compound of claim 1, wherein said chelating moiety is selected from the group consisting of DOTA (1,4,7,10-tetraazacyclododecane-1,4,7,10-tetraacetic acid), DOTMA (1R,4R,7R,10R)-α, α′, α″, α″′-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10-tetra propionic acid), DO3AM-acetic acid (2-(4,7,10-tris(2-amino-2-oxoethyl)-1,4,7,10-tetraazacyclododecan-1-yl)acetic acid), DOTA-GA anhydride (2,2′,2″-(10-(2,6-dioxotetrahydro-2H-pyran-3-yl)-1,4,7,10-tetraazacyclododecane-1,4,7-triyl)triacetic acid, DOTP (1,4,7,10-tetraazacyclododecane-1,4,7,10-tetra(methylene phosphonic acid)), DOTMP (1,4,6,10-tetraazacyclodecane-1,4,7,10-tetramethylene phosphonic acid, DOTA-4AMP (1,4,7,10-tetraazacyclododecane-1,4,7,10-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacyclotetradecane-1,4,8,11-tetrapropionic acid), TETA (1,4,8,11-tetraazacyclotetradecane-1,4,8,11-tetra acetic acid), HEHA (1,4,7,10,13,16-hexaazacyclohexadecane-1,4,7,10,13,16-hexaacetic acid), PEPA (1,4,7,10,13-pentaazacyclopentadecane-N,N′,N″,N″′,N″″′-pentaacetic acid), H₄octapa (N,N′-bis(6-carboxy-2-pyridylmethyl)-ethylenediamine-N,N′-diacetic acid), H₂dedpa (1,2-[[6-(carboxy)-pyridin-2-yl]-methylamino]ethane), H₆phospa (N,N′-(methylenephosphonate)-N,N′-[6-(methoxycarbonyl)pyridin-2-yl]-methyl-1,2-diaminoethane), TTHA (triethylenetetramine-N,N,N′,N″,N″′,N″′-hexaacetic acid), DO2P (tetraazacyclododecane dimethanephosphonic acid), HP-DO3A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), and porphyrin.

3. The compound of claim 2, wherein the compound is represented by Formula I-a, or a metal complex thereof: 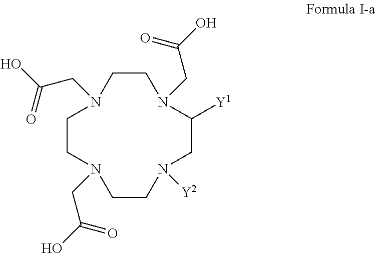
wherein Y¹ is —CH₂OCH₂(L²)ₙ-B, —C(O)(L²)ₙ-B, or —C(S)(L²)ₙ-B and Y² is —CH₂CO₂H; or
wherein Y¹ is H and Y² is L¹-(L²)ₙ-B.

4. The compound of any one of claims 1-3, wherein L¹ is 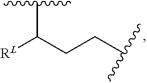
wherein R^{L} is hydrogen or —CO₂H.

5. The compound of any one of claims 1-4,
wherein the metal complex comprises a metal selected from the group consisting of Bi, Pb, Y, Mn, Cr, Fe, Co, Zn, Ni, Tc, In, Ga, Cu, Re, a lanthanide, and an actinide; or
wherein the metal complex comprises a radionuclide selected from the group consisting of ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga ⁶⁷Ga, ⁶⁸Ga ⁸²Rb, ⁸⁶Y, ⁸⁷Y, ⁸⁹Zr, ⁹⁰Y, ⁹⁷Ru, ⁹⁹Tc, ⁹⁹mTc, ¹⁰⁵Rh, ¹⁰⁹Pd, ¹¹¹In, ^{117m}Sn, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰¹Tl, ²⁰³Pb, ²¹¹At, ²¹²Pb, ²¹²Bi, ²¹³Bi, ²²³Ra, ²²⁵Ac, ²²⁷Th, and ²²⁹Th.

6. The compound of any one of claims 3-5, wherein Y¹ is H.

7. The compound of any one of claims 1-6, wherein X¹ is —C(O)NR¹—* or —NR¹C(O)—*, “*” indicating the attachment point to L³, and R¹ is H.

8. The compound of any one of claims 1-7, wherein Z¹ is —CH₂—.

9. The compound of any one of claims 1-8, wherein n is 1, and L³ comprises (CH₂CH₂O)₂₋₂₀.

10. The compound of any one of claims 1-8, wherein n is 1, and L³ is (CH₂CH₂O)ₘ(CH₂)_{w}, wherein m and w are each independently an integer between 0 and 10 (inclusive), and at least one of m and w is not 0.

11. The compound of claim 1, wherein the compound comprises one of the following structures, or a metal complex thereof:

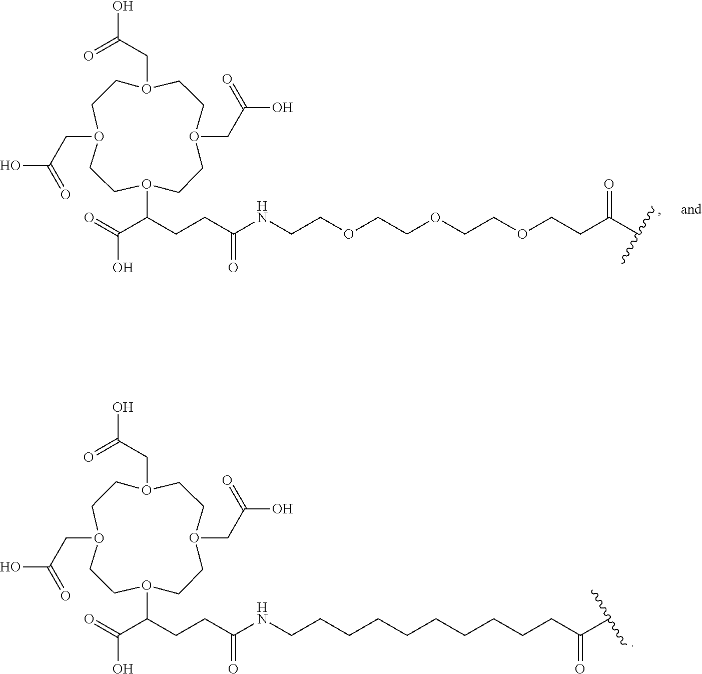

12. The compound of claim 1, wherein the compound comprises the following structure, or a metal complex thereof:

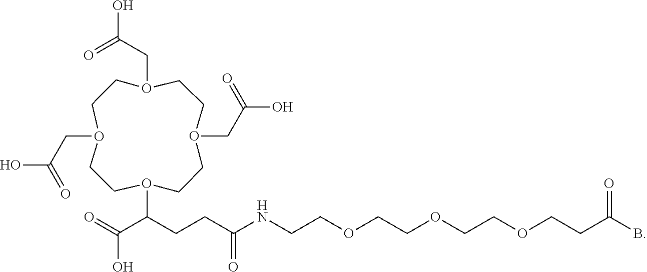

13. The compound of any one of claims 1-12, wherein A is a metal complex of a chelating moiety, and the metal complex comprises a radionuclide.

14. The compound of claim 13, wherein the radionuclide is ⁶⁸Ga, ¹¹¹In, ¹⁷⁷Lu, or ²²⁵Ac.

15. The compound of claim 13, wherein the radionuclide is ²²⁵Ac.

16. The compound of claim 13, wherein the radionuclide is an alpha emitter selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (¹⁴⁹Tb), or a progeny thereof.

17. The compound of claim 16, wherein the alpha emitter is ²²⁵Ac or a progeny thereof.

18. The compound of any one of the preceding claims, wherein the first antigen binding domain comprises:
i. a heavy chain variable (VH) region comprising the following complementarity determining regions (CDRs):
HCDR1 having the amino acid sequence of SEQ ID NO: 1
HCDR2 having the amino acid sequence of SEQ ID NO: 2
HCDR3 having the amino acid sequence of SEQ ID NO: 3; and
ii. a light chain variable (VL) region comprising the following CDRs:
LCDR1 having the amino acid sequence of SEQ ID NO: 4
LCDR2 having the amino acid sequence of SEQ ID NO: 5
LCDR3 having the amino acid sequence of SEQ ID NO: 6.

19. The compound of any one of the preceding claims, wherein the first antigen-binding domain is capable of binding to human EGFR with an affinity that is lower than the affinity with which an antigen-binding domain comprising the variable heavy region sequence and variable light region sequence of antibody QD6, the sequences set forth in SEQ ID NOs: 47 and 48, respectively is capable of binding to human EGFR.

20. The compound of any one of the preceding claims, wherein the first antigen binding domain comprises:
a. a VH region comprising an amino acid sequence having at least 70%, at least 80%, at least 90%, or at least 95% sequence identity to the amino acid sequence of SEQ ID NO: 15; and
b. a VL region comprising an amino acid sequence having at least 70%, at least 80%, at least 90%, or at least 95% sequence identity to the amino acid sequence of SEQ ID NO: 16.

21. The compound of any one of the preceding claims, wherein the second antigen binding domain comprises:
i. a VH region comprising the following CDRs:
HCDR1 having the amino acid sequence of SEQ ID NO: 17
HCDR2 having the amino acid sequence of SEQ ID NO: 18
HCDR3 having the amino acid sequence of SEQ ID NO: 19,
or a variant thereof in which one or two or three amino acids in one or more of HCDR1, HCDR2, and HCDR3 are substituted with another amino acid; and
ii. a VL region comprising the following CDRs:
LCDR1 having the amino acid sequence of SEQ ID NO: 20
LCDR2 having the amino acid sequence of SEQ ID NO: 21
LCDR3 having the amino acid sequence of SEQ ID NO: 22,
or a variant thereof in which one or two or three amino acids in one or more of LCDR1, LCDR2, and LCDR3 are substituted with another amino acid.

22. The compound of claim 21, wherein the second antigen binding domain comprises:
i. a VH region comprising the following CDRs:
HCDR1 having the amino acid sequence of SEQ ID NO: 17
HCDR2 having the amino acid sequence of SEQ ID NO: 18
HCDR3 having the amino acid sequence of SEQ ID NO: 19; and
ii. a VL region comprising the following CDRs:
LCDR1 having the amino acid sequence of SEQ ID NO: 20
LCDR2 having the amino acid sequence of SEQ ID NO: 21
LCDR3 having the amino acid sequence of SEQ ID NO: 22.

23. The compound of claim 21 or 22, wherein the second antigen binding domain comprises:
a. a VH region comprising an amino acid sequence having at least 70%, at least 80%, at least 90%, or at least 95% sequence identity to the amino acid sequence of SEQ ID NO: 31; and
b. a VL region comprising an amino acid sequence having at least 70%, at least 80%, at least 90%, or at least 95% sequence identity to the amino acid sequence of SEQ ID NO: 32.

24. The compound of any one of the preceding claims, wherein the antibody or antigen-binding fragment thereof comprises:
a. a first heavy chain, wherein the first heavy chain comprises the VH region of the first antigen-binding domain, and a first heavy chain constant (CH) region or a fragment thereof;
b. a first light chain, wherein the first light chain comprises the VL region of the first antigen-binding domain, and a first light chain constant (CL) region or a fragment thereof;
c. a second heavy chain, wherein the second heavy chain comprises the VH region of the second antigen-binding domain, and a second heavy chain constant (CH) region or a fragment thereof, and
d. a second light chain, wherein the second light chain comprises the VL region of the second antigen-binding domain, and a second light chain constant (CL) region or a fragment thereof.

25. The compound of claim 24, wherein the first and second CH region each comprise an amino acid sequence having at least 70%, at least 80%, at least 90%, or at least 95% sequence identity to the amino acid sequence of SEQ ID NO: 33.

26. The compound of claim 24 or 25, wherein the first and second heavy chain form a heterodimer, optionally wherein one of the first and second heavy chains comprises a cysteine (C) residue at position 354 and a tryptophan (W) residue at position 366 and the other heavy chain comprises a cysteine (C) residue at position 349, a valine (V) residue at position 407, a serine (S) at position 366 and an alanine (A) at position 368, wherein the numbering of the constant region is as per the EU index.

27. The compound of any one of claims 24-26, wherein the antibody or antigen-binding fragment thereof comprises:
a. a modified CH region, wherein the modified CH region comprises a substitution of a native non-cysteine amino acid to a cysteine amino acid; and
b. a modified corresponding CL region, wherein the modified CL comprises a substitution of a native non-cysteine amino acid to a cysteine amino acid, wherein either:
i. the first heavy chain comprises the modified CH region and the first light chain comprises the modified corresponding CL region; or
ii. the second heavy chain comprises the modified CH region and the second light chain comprises the modified corresponding CL region, and
wherein the substituted cysteine of the modified CH region and the substituted cysteine of the modified corresponding light chain can form a disulfide bond.

28. The compound of claim 27, wherein the modified CH region comprises a substitution of a native non-cysteine amino acid to a cysteine amino acid at position 126; and the modified corresponding CL region comprises a substitution of a native non-cysteine amino acid to a cysteine amino acid at position 121, wherein the numbering of the constant region is as per the EU index.

29. The compound of any one of claims 24-28, wherein the first and/or second CH region comprise a mutation to reduce or abrogate binding of the antibody or antigen-binding fragment thereof to one of more Fcγ receptors.

30. The compound of any one of claims 24-29, wherein the first and/or second CH region comprise a phenylalanine at position 234, glutamic acid at position 235, and serine at position 331, wherein the numbering of the constant region is as per the EU index.

31. The compound of any one of claims 24-30, wherein:
a. the first CH region comprises an amino acid sequence having at least 90%, 91%, 92%, 93%, 94%, 95%, 96%, 97%, 98%, 99%, or 100% sequence identity to the sequence set forth in SEQ ID NO: 39;
b. the second CH region comprises an amino acid sequence having at least 90%, 91%, 92%, 93%, 94%, 95%, 96%, 97%, 98%, 99%, or 100% sequence identity to the sequence set forth in SEQ ID NO: 40,
c. the first CL region comprises an amino acid sequence having at least 90%, 91%, 92%, 93%, 94%, 95%, 96%, 97%, 98%, 99%, or 100% sequence identity to the sequence set forth in SEQ ID NO: 41; and
d. the second CL region comprises an amino acid sequence having at least 90%, 91%, 92%, 93%, 94%, 95%, 96%, 97%, 98%, 99%, or 100% sequence identity to the sequence set forth in SEQ ID NO: 34.

32. The compound of any one of the preceding claims, wherein the antibody or antigen-binding fragment thereof comprises:
a. a first heavy chain comprising an amino acid sequence having at least 90%, 91%, 92%, 93%, 94%, 95%, 96%, 97%, 98%, 99%, or 100% sequence identity to the sequence set forth in SEQ ID NO: 35;
b. a second heavy chain comprising an amino acid sequence having at least 90%, 91%, 92%, 93%, 94%, 95%, 96%, 97%, 98%, 99%, or 100% sequence identity to the sequence set forth in SEQ ID NO: 36;
c. a first light chain comprising an amino acid sequence having at least 90%, 91%, 92%, 93%, 94%, 95%, 96%, 97%, 98%, 99%, or 100% sequence identity to the sequence set forth in SEQ ID NO: 37; and
d. a second light chain comprising an amino acid sequence having at least 90%, 91%, 92%, 93%, 94%, 95%, 96%, 97%, 98%, 99%, or 100% sequence identity to the sequence set forth in SEQ ID NO: 38.

33. The compound of claim 32, wherein:
a. the first heavy chain comprises an amino acid sequence having the sequence set forth in SEQ ID NO: 35;
b. the second heavy chain comprises an amino acid sequence having the sequence set forth in SEQ ID NO: 36;
c. the first light chain comprises an amino acid sequence having the sequence set forth in SEQ ID NO: 37; and
d. the second light chain comprises an amino acid sequence having the sequence set forth in SEQ ID NO: 38.

34. The compound of any one of the preceding claims, wherein:
a. the first antigen-binding domain that is capable of binding EGFR is capable of binding cynomologus EGFR;
b. the first antigen-binding domain that is capable of binding EGFR is capable of binding mouse EGFR;
c. the second antigen-binding domain that is capable of binding cMET is capable of binding cynomologus cMET;
d. the first antigen-binding domain is specific for EGFR;
e. the second antigen-binding domain is specific for cMET;
f. the antibody or antigen-binding fragment thereof is capable of concurrently engaging EGFR and cMET;
g. the antibody or antigen-binding fragment thereof is capable of being internalised into a cell;
h. the antibody or antigen-binding fragment thereof has cytotoxic activity when measured in an in vitro cell viability assay; and/or
i. the antibody or antigen-binding fragment thereof is capable of blocking ligand dependent signalling of EGFR and/or cMET.

35. The compound of claim 1, wherein:
(a) the first antigen-binding domain comprises:
i. a heavy chain variable (VH) region comprising the following complementarity determining regions (CDRs):
HCDR1 having the amino acid sequence of SEQ ID NO: 1
HCDR2 having the amino acid sequence of SEQ ID NO: 2
HCDR3 having the amino acid sequence of SEQ ID NO: 3; and
ii. a light chain variable (VL) region comprising the following CDRs:
LCDR1 having the amino acid sequence of SEQ ID NO: 4
LCDR2 having the amino acid sequence of SEQ ID NO: 5
LCDR3 having the amino acid sequence of SEQ ID NO: 6; and
(b) the second antigen-binding domain comprises:
i. a VH region comprising the following CDRs:
HCDR1 having the amino acid sequence of SEQ ID NO: 17
HCDR2 having the amino acid sequence of SEQ ID NO: 18
HCDR3 having the amino acid sequence of SEQ ID NO: 19; and
ii. a VL region comprising the following CDRs:
LCDR1 having the amino acid sequence of SEQ ID NO: 20
LCDR2 having the amino acid sequence of SEQ ID NO: 21
LCDR3 having the amino acid sequence of SEQ ID NO: 22.

36. The compound of any one of the preceding claims, wherein the first antigen-binding domain is capable of binding human EGFR with an affinity having a Kd that is:
a. between 10 and 100 nM;
b. between 20 and 80 nM;
c. between 30 and 75 nM; or
d. between 35 and 50 nM.

37. The compound of any one of the preceding claims, wherein the second antigen-binding domain is capable of binding human cMET with an affinity having a Kd that is:
a. lower than 10 nM; or
b. lower than 5 nM.

38. The compound of any one of claims 19, 36, and 37, wherein the affinity is measured by surface plasmon resonance.

39. The compound of claim 1, wherein the compound comprises the following structure: 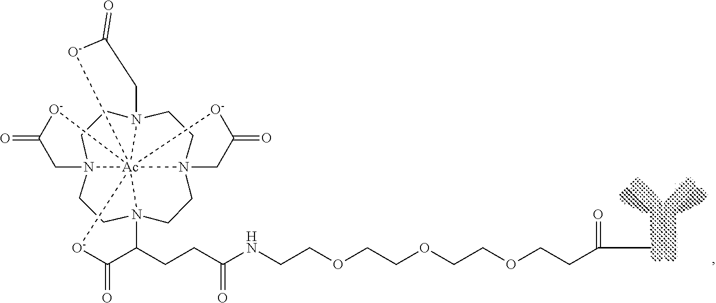
wherein 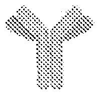
is an antibody or antigen-binding fragment thereof as defined in claim 1.

40. The compound of claim 39, wherein the antibody or antigen-binding fragment thereof is linked to A-L¹-(L²)ₙ- via the side-chain amino group of a lysine residue.

41. A pharmaceutical composition comprising a compound of any one of claims 1-40 and a pharmaceutically acceptable carrier, diluent, or excipient.

42. A method of treating cancer, said method comprising administering to a subject in need thereof a therapeutically effective amount a compound of any one of claims 1-40 or the composition of claim 41.

43. The method of claim 42, wherein the cancer is a solid tumor cancer selected from the group consisting of adrenocortical carcinoma, bladder cancer, breast cancer, cervical cancer, colorectal cancer, endometrial adenocarcinoma, Ewing's sarcoma, gallbladder carcinoma, glioma, head and neck cancer, liver cancer, lung cancer, neuroblastoma, neuroendocrine cancer, ovarian cancer, pancreatic cancer, prostate cancer, renal cell carcinoma, salivary adenoid cystic cancer, spermatocytic seminoma, and uveal melanoma.

44. The method of claim 43, wherein the cancer is lung cancer, colorectal cancer, pancreatic cancer, or head and neck cancer.

45. The method of any one of claims 42-44, further comprising administering an antiproliferative agent, a radiation sensitizer, or an immunomodulatory agent.

46. A compound of any one of claims 1-40, or the pharmaceutical composition of claim 41, for use in a method of treatment of cancer.

47. Use of a compound of any one of claims 1-40, or the pharmaceutical composition of claim 41, in the manufacture of a medicament for the treatment of cancer.


SEQUENCE LISTING (51 sequences)

SEQ ID NO: 1 - protein, 5 aa, synthetic construct
DNDFS

SEQ ID NO: 2 - protein, 17 aa, synthetic construct
AIVAVFRTETYAQKFQD

SEQ ID NO: 3 - protein, 15 aa, synthetic construct
RLMSAISGPGAPLLM

SEQ ID NO: 4 - protein, 14 aa, synthetic construct
TGTSSDVGGYNYVS

SEQ ID NO: 5 - protein, 7 aa, synthetic construct
DVSKRPS

SEQ ID NO: 6 - protein, 11 aa, synthetic construct
SSYTSSDTLEI

SEQ ID NO: 7 - protein, 30 aa, synthetic construct
QVQLVQSGAEVKKPGSSVKVSCKASGGTFS

SEQ ID NO: 8 - protein, 14 aa, synthetic construct
WVRQAPGQGLEWMG

SEQ ID NO: 9 - protein, 32 aa, synthetic construct
RVKITADISTRTTYMELSSLRSEDTAVYYCAR

SEQ ID NO: 10 - protein, 11 aa, synthetic construct
WGQGTLVTVSS

SEQ ID NO: 11 - protein, 22 aa, synthetic construct
QSALTQPRSVSGSPGQSVTISC

SEQ ID NO: 12 - protein, 15 aa, synthetic construct
WYQQHPGKAPKLMIY

SEQ ID NO: 13 - protein, 32 aa, synthetic construct
GVPDRFSGSKSGNTASLTISGLQAEDEADYYC

SEQ ID NO: 14 - protein, 10 aa, synthetic construct
FGGGTKLTVL

SEQ ID NO: 15 - protein, 124 aa, synthetic construct
QVQLVQSGAEVKKPGSSVKVSCKASGGTFSDNDFSWVRQAPGQGLEWMGAIVAVFRTETYAQKFQDRVKITADISTRTTYMELSSLRSEDTAVYYCARRLMSAISGPGAPLLMWGQGTLVTVSS

SEQ ID NO: 16 - protein, 111 aa, synthetic construct
QSALTQPRSVSGSPGQSVTISCTGTSSDVGGYNYVSWYQQHPGKAPKLMIYDVSKRPSGVPDRFSGSKSGNTASLTISGLQAEDEADYYCSSYTSSDTLEIFGGGTKLTVL

SEQ ID NO: 17 - protein, 5 aa, synthetic construct
DYYIH

SEQ ID NO: 18 - protein, 17 aa, synthetic construct
WMNPNSGNTGYAQKFQG

SEQ ID NO: 19 - protein, 7 aa, synthetic construct
GQGYTHS

SEQ ID NO: 20 - protein, 11 aa, synthetic construct
RASEGIYHWLA

SEQ ID NO: 21 - protein, 7 aa, synthetic construct
KASSLAS

SEQ ID NO: 22 - protein, 9 aa, synthetic construct
QQYSNYPPT

SEQ ID NO: 23 - protein, 30 aa, synthetic construct
QVQLVQSGAEVKKPGASVKVSCKASGYTFT

SEQ ID NO: 24 - protein, 14 aa, synthetic construct
WVRQATGQGLEWMG

SEQ ID NO: 25 - protein, 32 aa, synthetic construct
RVTMTRDTSISTAYMELSSLRSEDTAVYYCAR

SEQ ID NO: 26 - protein, 11 aa, synthetic construct
WGQGTMVTVSS

SEQ ID NO: 27 - protein, 23 aa, synthetic construct
DIQMTQSPSTLSASVGDRVTITC

SEQ ID NO: 28 - protein, 15 aa, synthetic construct
WYQQKPGKAPKLLIY

SEQ ID NO: 29 - protein, 32 aa, synthetic construct
GVPSRFSGSGSGTEFTLTISSLQPDDFATYYC

SEQ ID NO: 30 - protein, 10 aa, synthetic construct
FGGGTKLEIK

SEQ ID NO: 31 - protein, 116 aa, synthetic construct
QVQLVQSGAEVKKPGASVKVSCKASGYTFTDYYIHWVRQATGQGLEWMGWMNPNSGNTGYAQKFQGRVTMTRDTSISTAYMELSSLRSEDTAVYYCARGQGYTHSWGQGTMVTVSS

SEQ ID NO: 32 - protein, 107 aa, synthetic construct
DIQMTQSPSTLSASVGDRVTITCRASEGIYHWLAWYQQKPGKAPKLLIYKASSLASGVPSRFSGSGSGTEFTLTISSLQPDDFATYYCQQYSNYPPTFGGGTKLEIK

SEQ ID NO: 33 - protein, 330 aa, synthetic construct [sequence omitted]

SEQ ID NO: 34 - protein, 107 aa, synthetic construct [sequence omitted]

SEQ ID NO: 35 - protein, 454 aa, synthetic construct
QVQLVQSGAEVKKPGSSVKVSCKASGGTFSDNDFSWVRQAPGQGLEWMGAIVAVFRTETYAQKFQDRVKITADISTRTTYMELSSLRSEDTAVYYCARRLMSAISGPGAPLLMWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 446 aa, synthetic construct
QVQLVQSGAEVKKPGASVKVSCKASGYTFTDYYIHWVRQATGQGLEWMGWMNPNSGNTGYAQKFQGRVTMTRDTSISTAYMELSSLRSEDTAVYYCARGQGYTHSWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 37 - protein, 217 aa, synthetic construct
QSALTQPRSVSGSPGQSVTISCTGTSSDVGGYNYVSWYQQHPGKAPKLMIYDVSKRPSGVPDRFSGSKSGNTASLTISGLQAEDEADYYCSSYTSSDTLEIFGGGTKLTVLGQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 38 - protein, 214 aa, synthetic construct
DIQMTQSPSTLSASVGDRVTITCRASEGIYHWLAWYQQKPGKAPKLLIYKASSLASGVPSRFSGSGSGTEFTLTISSLQPDDFATYYCQQYSNYPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 330 aa, synthetic construct
ASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 41 - protein, 106 aa, synthetic construct
GQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 42 - protein, 621 aa, synthetic construct [sequence omitted]

SEQ ID NO: 43 - protein, 908 aa, synthetic construct
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFT

SEQ ID NO: 44 - protein, 908 aa, synthetic construct
ECKEALAKSEMNVNMKYQLPNFTAETAIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPNQCPDCVVSALGAKVLSSVKDRFINFFVGNTINSSYFPHHPLHSISVRRLKETKDGFMFLTDQSYIDVLPEFRDSYPIKYIHAFESNNFIYFLTVQRETLNAQTFHTRIIRFCSLNSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRAEFTTALQRVDLFMGQFSEVLLTSISTFVKGDLTIANLGTSEGRFMQVVVSRSGPSTPHVNFLLDSHPVSPEVIVEHPLNQNGYTLVVTGKKITKIPLNGLGCRHFQSCSQCLSAPPFVQCGWCHDKCVRSEECPSGTWTQQICLPAIYKVFPTSAPLEGGTRLTICGWDFGFRRNNKFDLKKTRVLLGNESCTLTLSESTMNTLKCTVGPAMNKHFNMSIIISNGHGTTQYSTFSYVDPIITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLHSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFT

SEQ ID NO: 45 - skipped

SEQ ID NO: 46 - protein, 621 aa, synthetic construct
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSEFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDTLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSSQKTKIISNRGENSCKATGQVCHALCSPEGCWGPEPRDCVSCQNVSRGRECVDKCNILEGEPREFVENSECIQCHPECLPQVMNITCTGRGPDNCIQCAHYIDGPHCVKTCPAGVMGENNTLVWKYADAGHVCHLCHPNCTYGCTGPGLEGCARNGPKIPS

SEQ ID NO: 47 - protein, 124 aa, synthetic construct
QVQLVQSGAEVKKPGSSVKVSCKASGGTFSDNDFSWVRQAPGQGLEWMGAIVAVVRTETYAQKFQDRVKITADISTRTTYMELSSLRSEDTAVYYCARRLMSAISGPGAPLLMWGQGTLVTVSS

SEQ ID NO: 48 - protein, 111 aa, synthetic construct
QSALTQPRSVSGSPGQSVTISCTGTSSDVGGYNYVSWYQQHPGKAPKLMIYDVSERPSGVPDRFSGSKSGNTASLTISGLQAEDEADYYCFSYTSSDTLEIFGGGTKLTVL

SEQ ID NO: 49 - protein, 19 aa, Homo sapiens
STWWKEPLNIVSFLFCFAS

SEQ ID NO: 50 - protein, 10 aa, Homo sapiens
RHVNIALIQR

SEQ ID NO: 51 - protein, 5 aa, synthetic construct
  VARIANT 4: note = Any amino acid
LPTXG